(19) United States
(12) Patent Application Publication
(10) Pub. No.: US 2010/0297676 A1
(43) Pub. Date: Nov. 25, 2010

(54) METHODS FOR DIAGNOSIS, PROGNOSIS AND METHODS OF TREATMENT

(75) Inventors: Wendy J. Fantl, San Francisco, CA (US); Alessandra Cesano, Redwood City, CA (US); Erik Evensen, South San Francisco, CA (US); Adam Palazzo, San Francisco, CA (US)

Correspondence Address: WILSON, SONSINI, GOODRICH & ROSATI / NODALITY, INC, 650 Page Mill Road, Palo Alto, CA, 94304-1050, US

(73) Assignee: Nodality, Inc., South San Francisco, CA (US)

(21) Appl. No.: 12/784,478

(22) Filed: May 20, 2010

Related U.S. Application Data
(60) Provisional application No. 61/306,872, filed on Feb. 22, 2010.
(60) Provisional application No. 61/306,665, filed on Feb. 22, 2010.
(60) Provisional application No. 61/263,281, filed on Nov. 20, 2009.
(60) Provisional application No. 61/241,773, filed on Sep. 11, 2009.
(60) Provisional application No. 61/216,825, filed on May 20, 2009.

(51) Int. Cl. G01N 33/53; C12Q 1/48; C12Q 1/37; C12Q 1/02

(52) U.S. Cl. 435/7.24; 435/15; 435/23; 435/29

(57) ABSTRACT

This invention is directed to methods and compositions for diagnosis, prognosis and for determining methods of treatment. The physiological status of a cell present in a sample (e.g. clinical sample) can be used in diagnosis or prognosis of a condition (e.g. Chronic Lymphocytic Leukemia), in patient selection for therapy, to monitor treatment and to modify or optimize therapeutic regimens.

### CROSS-REFERENCE

This application claims the benefit of U.S. Provisional Application No. 61/306,872, filed Feb. 22, 2010, U.S. Provisional Application No. 61/306,665, filed Feb. 22, 2010, U.S. Provisional Application No. 61/263,281, filed Nov. 20, 2009, U.S. Provisional Application No. 61/241,773, filed Sep. 11, 2009, and U.S. Provisional Application No. 61/216,825, filed May 20, 2009, all of which applications are incorporated herein by reference.

### BACKGROUND OF THE INVENTION

Many conditions are characterized by disruptions in cellular pathways that lead, for example, to aberrant control of cellular processes, or to uncontrolled growth and proliferation of cells. These disruptions are often caused by changes in the activity of molecules participating in cellular pathways. For example, specific signaling pathway alterations have been described for many cancers. Despite the increasing evidence that disruption in cellular pathways mediate the detrimental transformation, the precise molecular events underlying these transformations have not been elucidated. As a result, therapeutics may not be effective in treating conditions involving cellular pathways that are not well understood. Thus, the successful diagnosis of a condition and use of therapies will require knowledge of the cellular events that are responsible for the condition pathology.

In addition, patients suffering from different conditions follow heterogeneous clinical courses. For instance, tremendous clinical variability among remissions is also observed in cancer patients, even those that occur after one course of therapy. Some leukemia patients survive for prolonged periods without definitive therapy, while others die rapidly despite aggressive treatment. Patients who are resistant to therapy have very short survival times, regardless of when the resistance occurs. While various staging systems have been developed to address this clinical heterogeneity, they cannot accurately predict whether an early or intermediate stage patient will experience an indolent or aggressive course of disease.

Accordingly, there is a need for a reliable indicator of an individual predicted disease course to help clinicians to identify those patients that will respond to treatment, patients that progress to a more advanced state of the disease and patients with emerging resistance to treatment.

### SUMMARY OF THE INVENTION

As disclosed herein is a method for classifying a cell comprising contacting the cell with a modulator or an inhibitor used to determine the presence or absence of a change in activation level of an activatable element in the cell, and classifying the cell based on the presence or absence of the change in the activation level of the activatable element. In some embodiments the change in activation level of an activatable element is an increase in the activation level of an activatable element. In some embodiments the activatable element is a protein subject to phosphorylation or dephosphorylation.

In some embodiments, one aspect of the invention is tyrosine phosphatase inhibitor (e.g. peroxide) mediated STAT5 or AKT phosphorylation to segregate or stratify patients. Other examples of tyrosine phosphatase inhibitors include CD45-associated protein tyrosine phosphatase inhibitor, PHPS1, PP1, PP2, Bay U6751, BVT.948, NSC 295642, PRL-3 Inhibitor I, Phenylarsine oxide, Sodium Stibogluconate, Sodium orthovanadate, pervanadate, bisperoxovanadium, phenylarsine oxide, alendronate, etidronate, vanadate, gallium nitrate, suramin, and aplidin. In another embodiment, the invention relates to measuring in vitro apoptosis in response to F-ara-A into separate classes of patients who are apoptosis competent or refractory. Another aspect of the invention relates to the use of classification and modeling methods such as logistic regression (including regularized, penalized, and shrinkage methods including lasso and ridge), decision trees, random forests, support vector machines, boosting, etc. to generate univariate and multivariate models associating tyrosine phosphatase inhibitor (e.g. hydrogen peroxide (H₂O₂)) or B-cell receptor cross linking induced changes in phosphorylation with the ability of cells to undergo apoptosis. Another aspect of the invention is the detection of ZAP-70 to increase the predictability of the area under the ROC curve or the use of the ROC curve to determine the suitability of a classification and modeling method. Another aspect of the invention relates to the use of mixture models to represent data for the uses disclosed herein. In another embodiment, detection of ZAP-70, IGVH and/or CD38 can be used as clinical covariates that can be combined with phosphorylation and/or signaling readouts, in multivariate models of the methods described throughout the specification.

In some embodiments of the methods, the invention provides a method for classifying a cell by contacting the cell with an inhibitor; determining the activation levels of a plurality of activatable elements in the cell; and classifying the cell based on the activation level. In some embodiments, the inhibitor is a kinase or phosphatase inhibitor, such as adaphostin, AG 490, AG 825, AG 957, AG 1024, aloisine, aloisine A, alsterpaullone, aminogenistein, API-2, apigenin, arctigenin, AY-22989, BAY 61-3606, bisindolylmaleimide IX, chelerythrine, 10-[4′-(N,N-Diethylamino)butyl]-2-chlorophenoxazine hydrochloride, dasatinib, 2-Dimethylamino-4,5,6,7-tetrabromo-1H-benzimidazole, 5,7-Dimethoxy-3-(4-pyridinyl)quinoline dihydrochloride, edelfosine, ellagic acid, enzastaurin, ER 27319 maleate, erlotinib, ET18OCH3, fasudil, flavopiridol, gefitinib, GW 5074, H-7, H-8, H-89, HA-100, HA-1004, HA-1077, HA-1100, hydroxyfasudil, indirubin-3′-oxime, 5-Iodotubercidin, kenpaullone, KN-62, KY12420, LFM-A13, lavendustin A, luteolin, LY-294002, LY294002, mallotoxin, ML-9, NSC-154020, NSC-226080, NSC-231634, NSC-664704, NSC-680410, NU6102, olomoucine, oxindole I, PD-153035, PD-98059, PD-169316, phloretin, phloridzin, piceatannol, picropodophyllin, PK1, PP1, PP2, purvalanol A, quercetin, R406, R788, rapamune, rapamycin, Ro 31-8220, roscovitine, rottlerin, SB202190, SB203580, sirolimus, sorafenib, SL327, SP600125, staurosporine, STI-571, SU-11274, SU1498, SU4312, SU6656, 4,5,6,7-Tetrabromotriazole, TG101348, Triciribine, Tyrphostin AG 490, Tyrphostin AG 825, Tyrphostin AG 957, Tyrphostin AG 1024, Tyrphostin SU1498, U0126, VX-509, VX-667, VX-680, W-7, wortmannin, XL-019, XL-147, XL-184, XL-228, XL-281, XL-518, XL-647, XL-765, XL-820, XL-844, XL-880, Y-27632, ZD-1839, ZM-252868, ZM-447-439, H₂O₂, siRNA, miRNA, Cantharidin, (−)-p-Bromotetramisole, Microcystin LR, Sodium Orthovanadate, Sodium Pervanadate, Vanadyl sulfate, Sodium oxodiperoxo(1,10-phenanthroline)vanadate, bis(maltolato)oxovanadium(IV), Sodium Molybdate, Sodium Perm olybdate, Sodium Tartrate, Imidazole, Sodium Fluoride, β-Glycerophosphate, Sodium Pyrophosphate Decahydrate, Calyculin A, Discodermia calyx, bpV(phen), mpV(pic), DMHV, Cypermethrin, Dephostatin, Okadaic Acid, NIPP-1, N-(9,10-Dioxo-9,10-dihydro-phenanthren-2-yl)-2,2-dimethyl-propionamide, α-Bromo-4-hydroxyacetophenone, 4-Hydroxyphenacyl Br, α-Bromo-4-methoxyacetophenone, 4-Methoxyphenacyl Br, α-Bromo-4-(carboxymethoxy)acetophenone, 4-(Carboxymethoxy)phenacyl Br, and bis(4-Trifluoromethylsulfonamidophenyl)-1,4-diisopropylbenzene, phenyarsine oxide, Pyrrolidine Dithiocarbamate, or Aluminum fluoride. In some embodiments the phosphatase inhibitor is a tyrosine phosphatase inhibitor, such as H₂O₂.

In some embodiments the cell or cell population (hereinafter called a “cell”) is a hematopoietic-derived cell. In some embodiments, the hematopoietically derived cell is selected from the group consisting of pluripotent hematopoietic stem cells, B-lymphocyte lineage progenitor or derived cells, T-lymphocyte lineage progenitor or derived cells, NK cell lineage progenitor or derived cells, granulocyte lineage progenitor or derived cells, monocyte lineage progenitor or derived cells, megakaryocyte lineage progenitor or derived cells and erythroid lineage progenitor or derived cells. In some embodiments, the hematopoietic derived cell is a B-lymphocyte lineage progenitor and derived cell, e.g., an early pro-B cell, late pro-B cell, large pre-B cell, small pre-B cell, immature B cell, mature B cell, plasma cell and memory B cell, a CD5+ B cell, a CD38+ B cell, a B cell bearing a mutated or non mutated heavy chain of the B cell receptor, or a B cell expressing ZAP-70.

In some embodiments, the classification or correlation includes classifying the cell as a cell that is correlated with a clinical outcome. In some embodiments, the clinical outcome is the prognosis and/or diagnosis of a condition. In some embodiments, the clinical outcome is the presence or absence of a neoplastic, autoimmune or a hematopoietic condition, such as Non-Hodgkin Lymphoma, Hodgkin or other lymphomas, acute or chronic leukemias, polycythemias, thrombocythemias, multiple myeloma or plasma cell disorders, e.g., amyloidosis and Waldenstrom's macroglobulinemia, myelodysplastic disorders, myeloproliferative disorders, myelofibrosis, or atypical immune lymphoproliferations, systemic lupus erythematosis (SLE), rheumatoid arthritis (RA). In some embodiments, the neoplastic, autoimmune or hematopoietic condition is non-B lineage derived, such as acute myeloid leukemia (AML), Chronic Myeloid Leukemia (CML), non-B cell acute lymphocytic leukemia (ALL), non-B cell lymphomas, myelodysplastic disorders, myeloproliferative disorders, myelofibrosis, thrombocythemias, or non-B atypical immune lymphoproliferations. In some embodiments, the neoplastic, autoimmune or hematopoietic condition is a B-Cell or B cell lineage derived disorder, such as Chronic Lymphocytic Leukemia (CLL), B-cell lymphoma, B lymphocyte lineage leukemia, B lymphocyte lineage lymphoma, Multiple Myeloma, acute lymphoblastic leukemia (ALL), B-cell pro-lymphocytic leukemia, precursor B lymphoblastic leukemia, hairy cell leukemia or plasma cell disorders, e.g., amyloidosis or Waldenstrom's macroglobulinemia, B cell lymphomas including but not limited to diffuse large B cell lymphoma, follicular lymphoma, mucosa associated lymphatic tissue lymphoma, small cell lymphocytic lymphoma and mantle cell lymphoma. In some embodiments, the condition is CLL. In some embodiments, the CLL is defined by a monoclonal B cell population that co-expresses CD5 with CD19 and CD23 or CD5 with CD20 and CD23 and by surface immunoglobulin expression.

In some embodiments, the clinical outcome is the staging or grading of a neoplastic, autoimmune or hematopoietic condition. Examples of staging in methods provided by the invention include aggressive, indolent, benign, refractory, Roman Numeral staging, TNM Staging, Rai staging, Binet staging, WHO classification, FAB classification, IPSS score, WPSS score, limited stage, extensive stage, staging according to cellular markers such as ZAP-70 and CD38, occult, including information that may inform on time to progression, progression free survival, overall survival, or event-free survival.

In some embodiments of the invention, the activation level of the plurality of activatable elements in the cell is selected from the group consisting of cleavage by extracellular or intracellular protease exposure, novel hetero-oligomer formation, glycosylation level, phosphorylation level, acetylation level, methylation level, biotinylation level, glutamylation level, glycylation level, hydroxylation level, isomerization level, prenylation level, myristoylation level, lipoylation level, phosphopantetheinylation level, sulfation level, ISGylation level, nitrosylation level, palmitoylation level, SUMOylation level, ubiquitination level, neddylation level, citrullination level, deamidation level, disulfide bond formation level, proteolytic cleavage level, translocation level, changes in protein turnover, multi-protein complex level, oxidation level, multi-lipid complex, and biochemical changes in cell membrane. In some embodiments, the activation level is a phosphorylation level. In some embodiments, the activatable element is selected from the group consisting of proteins, carbohydrates, lipids, nucleic acids and metabolites. In some embodiments, the activatable element is a protein. In some embodiments, the activatable element is a change in metabolic state, temperature, or local ion concentration. In embodiments where the activatable element is a protein, in some embodiments the protein is a protein subject to phosphorylation or dephosphorylation, such as kinases, phosphatases, adaptor/scaffold proteins, ubiquitination enzymes, adhesion molecules, contractile proteins, cytoskeletal proteins, heterotrimeric G proteins, small molecular weight GTPases, guanine nucleotide exchange factors, GTPase activating proteins, caspases and proteins involved in apoptosis (e.g. PARP), ion channels, molecular transporters, molecular chaperones, metabolic enzymes, vesicular transport proteins, hydroxylases, isomerases, transferases, deacetylases, methylases, demethylases, proteases, esterases, hydrolases, DNA binding proteins or transcription factors. In some embodiments, the protein is selected from the group consisting of PI3-Kinase (p85, p110a, p110b, p110d), Jak1, Jak2, SOCs, Rac, Rho, Cdc42, Ras-GAP, Vav, Tiam, Sos, Dbl, Nck, Gab, PRK, SHPT, and SHP2, SHIP1, SHIP2, sSHIP, PTEN, Shc, Grb2, PDK1, SGK, Akt1, Akt2, Akt3, TSC1,2, Rheb, mTor, 4EBP-1, p70S6Kinase, S6, LKB-1, AMPK, PFK, Acetyl-CoAa Carboxylase, DokS, Rafs, Mos, Tp12, MEK1/2, MLK3, TAK, DLK, MKK3/6, MEKK1,4, MLK3, ASK1, MKK4/7, SAPK/JNK1,2,3, p38s, Erk1/2, Syk, Btk, BLNK, LAT, ZAP-70, Lyn, Cbl, SLP-76, PLCγ₁, PLCγ2, transcription factor, STAT1, STAT3, STAT4, STAT5, STATE, FAK, p130CAS, PAKs, LIMK1/2, Hsp90, Hsp70, Hsp27, SMADs, Rel-A (p65-NFκB), CREB, Histone H2B, HATs, HDACs, PKR, Rb, Cyclin D, Cyclin E, Cyclin A, Cyclin B, P16, p14Arf, p27KIP, p21CIP, Cdk4, Cdk6, Cdk7, Cdk1, Cdk2, Cdk9, Cdc25, A/B/C, Abl, E2F, FADD, TRADD, TRAF2, RIP, Myd88, BAD, Bcl-2, Mcl-1, Bcl-XL, Caspase 2, Caspase 3, Caspase 6, Caspase 7, Caspase 8, Caspase 9, PARP, IAPB, Smac, Fodrin, Actin, Src, Lyn, Fyn, Lyn, NIK, IKB, p65(RelA), IKKα, PKA, PKCa, PKCα, PKCβ, PKCθ, CAMδ, Elk, AFT, Myc, Egr-1, NFAT, ATF-2, Mdm2, p53, DNA-PK, Chk1, Chk2, ATM, ATR, \tilde{β}catenin, CrkL, GSK3α, GSK3β, and FOXO. In some embodiments, the protein selected from the group consisting of Erk, Syk, ZAP-70, Lyn, Btk, BLNK, Cbl, PLCγ2, Akt, RelA, p38, S6. In some embodiments the protein is S6.

In some embodiments, the protein is selected from the group consisting of HER receptors, PDGF receptors, Kit receptor, FGF receptors, Eph receptors, Trk receptors, IGF receptors, Insulin receptor, Met receptor, Ret, VEGF receptors, TIE1, TIE2, FAK, Jak1, Jak2, Jak3, Tyk2, Src, Lyn, Fyn, Lyn, Fgr, Yes, Csk, Abl, Btk, ZAP-70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Weel, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3α, GSK3β, Cdks, CLKs, PKR, PI3-Kinase class 1, class 2, class 3, mTor, SAPK/JNK1,2,3, p38s, PKR, DNA-PK, ATM, ATR, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, SHIPs, myotubularins, phosphoinositide kinases, phospholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, IL-2, IL-4, IL-8, IL-6, interferon γ, interferon α, suppressors of cytokine signaling (SOCs), Cbl, SCF ubiquitination ligase complex, APC/C, adhesion molecules, integrins, Immunoglobulin-like adhesion molecules, selectins, cadherins, catenins, focal adhesion kinase, p130CAS, fodrin, actin, paxillin, myosin, myosin binding proteins, tubulin, eg5/KSP, CENPs, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, Vav, Tiam, Sos, Dbl, PRK, TSC1,2, Ras-GAP, Arf-GAPs, Rho-GAPs, caspases, Caspase 2, Caspase 3, Caspase 6, Caspase 7, Caspase 8, Caspase 9, PARP, Bcl-2, Mcl-1, Bcl-XL, Bcl-w, Bcl-B, A1, Bax, Bak, Bok, Bik, Bad, Bid, Bim, Bmf, Hrk, Noxa, Puma, IAPB, XIAP, Smac, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, Pin1 prolyl isomerase, topoisomerases, deacetylases, Histone deacetylases, sirtuins, histone acetylases, CBP/P300 family, MYST family, ATF2, DNA methyl transferases, Histone H3K4 demethylases, H3K27, JHDM2A, UTX, VHL, WT-1, p53, Hdm, PTEN, ubiquitin proteases, urokinase-type plasminogen activator (uPA) and uPA receptor (uPAR) system, cathepsins, metalloproteinases, esterases, hydrolases, separase, potassium channels, sodium channels, multi-drug resistance proteins, P-Glycoprotein, nucleoside transporters, Ets, Elk, SMADs, Rel-A (p65-NFKB), CREB, NFAT, ATF-2, AFT, Myc, Fos, Sp1, Egr-1, T-bet, β-catenin, HIFs, FOXOs, E2Fs, SRFs, TCFs, Egr-1, β-catenin, FOXO transcription factor, STAT1, STAT2, STAT3, STAT4, STAT5a, STAT5b, STATE, p53, WT-1, HMGA, pS6, 4EPB-1, eIF4E-binding protein, RNA polymerase, initiation factors, elongation factors.

In some embodiments of the methods of the invention, the modulator to which the cell is subjected is an activator or an inhibitor. In some embodiments, the modulator is, e.g., a growth factor, cytokine, adhesion molecule modulator, hormone, small molecule, polynucleotide, antibodies, natural compounds, lactones, chemotherapeutic agents, immune modulator, carbohydrate, proteases, ions, reactive oxygen species, or radiation. In some embodiments, the modulator is a B cell receptor modulator, e.g., a B cell receptor activator such as a cross-linker of the B cell receptor complex or the B-cell co-receptor complex. In some embodiments of the invention, the cell is subjected to a modulator and a separate B cell receptor modulator (such as a B cell receptor cross-linker). In some embodiments, the cross-linker is an antibody, or molecular binding entity. In some embodiments, the cross-linker is an antibody, such as a multivalent antibody. In some embodiments, the antibody is a monovalent, bivalent, or multivalent antibody made more multivalent by attachment to a solid surface or tethered on a nanoparticle surface to increase the local valency of the epitope binding domain. In some embodiments, the cross-linker is a molecular binding entity, such as an entity that acts upon or binds the B cell receptor complex via carbohydrates or an epitope in the complex. In some embodiments, the molecular binding entity is a monovalent, bivalent, or multivalent binding entity that is made more multivalent by attachment to a solid surface or tethered on a nanoparticle surface to increase the local valency of the epitope binding domain. In some embodiments where the modulator is a B cell receptor modulator, e.g., a B cell receptor activator such as a cross-linker of the B cell receptor complex or the B-cell co-receptor complex, cross-linking includes binding of an antibody or molecular binding entity to the cell and then causing its crosslinking via interaction of the cell with a solid surface that causes crosslinking of the BCR complex via antibody or molecular binding entity. In some embodiments, the crosslinker is selected from the group consisting of F(ab)₂ IgM, IgG, IgD, polyclonal BCR antibodies, monoclonal BCR antibodies, and Fc receptor derived binding elements. The Ig may be derived from a species selected from the group consisting of mouse, goat, rabbit, pig, rat, horse, cow, shark, chicken, or llama. In some embodiments, the crosslinker is F(ab)₂ IgM, Polyclonal IgM antibodies, Monoclonal IgM antibodies, Biotinylated F(ab)₂ IgG/M, Biotinylated Polyclonal IgM antibodies, Biotinylated Monoclonal IgM antibodies and/or a combination thereof.

In some embodiments of the methods of the invention, the cell is subjected to a B cell receptor activator and a phosphatase inhibitor or kinase inhibitor, such as F(ab)₂IgM or biotinylated F(ab)₂IgM and a phosphatase inhibitor (e.g., H₂0₂).

In some embodiments, the invention provides a method of determining a tonic signaling (ligand independent) status of a cell by subjecting the cell to a modulator, determining the activation level of an activatable element that participates in a tonic signaling pathway in the cell, and determining the status of a tonic signaling pathway in the cell from the activation level. In some embodiments, a condition of an individual is determined based on tonic signaling status of a cell. In some embodiments, the condition is a neoplastic, autoimmune and/or hematopoietic condition as discussed above.

In some embodiments, the tonic signaling status of a cell is correlated with a clinical outcome such as prognosis or diagnosis of the condition.

In some embodiments, the correlation is determining the individual's response to a treatment, e.g., normal responder, hyper responder, poor responder, having emerging resistance, non-compliant, and adverse reaction.

In some embodiments of this aspect, the invention provides a method of correlating an activation level of a B-lymphocyte lineage derived cell with a neoplastic, autoimmune or hematopoietic condition in an individual by subjecting the B-lymphocyte lineage derived cell from the individual to a modulator; determining the activation levels of a plurality of activatable elements that participate in a tonic signaling pathway in the B-lymphocyte lineage derived cell; and identifying a pattern of the activation levels of the plurality of activatable elements in the tonic signaling pathway in the cell that correlates with a clinical outcome, such as the prediction of outcome for a particular treatment, a prognosis or diagnosis of a certain condition (e.g., a neoplastic condition).

In some embodiments of the methods of the invention, the cell is further subjected to a second modulator, e.g., the cell may be subjected to a B cell receptor activator and a phosphatase inhibitor, such as F(ab)₂IgM or biotinylated F(ab)₂IgM and a phosphatase inhibitor (e.g., H₂0₂).

In addition to determining the activation level of an activatable protein, in some embodiments the methods for classifying a cell further comprise determining the level of an additional intracellular marker and/or a cell surface marker. In some embodiments the methods for classifying a cell comprise determining the level of an additional intracellular marker. In some embodiments the intracellular marker is a captured intracellular cytokine. In some embodiments the methods for classifying a cell comprise determining the level of an additional cell surface marker. In some embodiments the cell surface marker is a cell surface ligand or receptor. In some embodiments the cell surface marker is a component of a B-cell receptor complex. In some embodiments the cell surface marker is CD45, CD5, CD19, CD20, CD22, CD23, CD27, CD37, CD40, CD52, CD79, CD38, CD96, major histocompatability antigen (MHC) Class 1 or MHC Class 2.

In some embodiments the methods of the invention for prognosis, diagnosis, or determination of treatment further comprise determining the level of an additional serum marker. In some embodiments the serum marker comprises a protein. In some embodiments the serum marker is a cytokine, growth factor, chemokine, soluble receptor, small compound, or pharmaceutical drug. In some embodiments the serum marker comprises a component or product of a pathogen or parasite. In some embodiments the serum marker is selected from a group consisting of beta-2-microglobulin, calcitonin, thymidine kinase and ferritin.

In some embodiments, the invention provides a method of correlating an activation level of B-lymphocyte lineage derived cells with a neoplastic, autoimmune or hematopoietic condition in an individual by subjecting the B-lymphocyte lineage derived cell from the individual to a modulator; determining the activation levels of a plurality of activatable elements in the B-lymphocyte lineage derived cell; and identifying a pattern of the activation levels of the plurality of activatable elements in the cell that correlates with the neoplastic condition. In some embodiments, the activatable element is selected from the group consisting of elements selected from the group consisting of Erk, Syk, ZAP-70, Lyn, Btk, BLNK, Cbl, PLCγ2, Akt, RelA, p38, S6 (which can be phosphorylated). In some embodiments, the activatable element is selected from the group consisting of Cbl, PLCγ2, and S6. In some embodiments, the activatable element is S6. In some embodiments, the B-lymphocyte lineage progenitor or derived cell is selected from the group consisting of early pro-B cell, late pro-B cell, large pre-B cell, small pre-B cell, immature B cell, mature B cell, plasma cell and memory B cell, a CD5+ B cell, a CD38+ B cell, a B cell bearing a mutilated or non mutated heavy chain of the B cell receptor, or a B cell expressing ZAP-70. In some embodiments, the invention provides methods for correlating and/or classifying an activation state of a CLL cell with a clinical outcome in an individual by subjecting the CLL cell from the individual to a modulator, where the CLL cell expresses a B-Cell receptor (BCR), determining the activation levels of a plurality of activatable elements, and identifying a pattern of the activation levels of the plurality of activatable elements to determine the presence or absence of an alteration in signaling proximal to the BCR, wherein the presence of the alteration is indicative of a clinical outcome.

In some embodiments the method comprises identifying a pattern of said activation levels of said plurality of activatable elements in said cell, wherein said pattern is correlated to a disease or condition.

In some embodiments, the correlation is determining the individual's response to a specific treatment, e.g., normal responder, hyper responder, poor responder, having emerging resistance, non-compliant, and adverse reaction.

In some embodiments of the invention, the modulator to which the cell is subjected is an activator or an inhibitor. In some embodiments, the modulator is, e.g., a growth factor, cytokine, adhesion molecule modulator, hormone, small molecule, polynucleotide, antibody, natural compound, lactone, chemotherapeutic agent, immune modulator, carbohydrate, protease, ion, reactive oxygen species, or radiation. In some embodiments, the modulator is an antibody, e.g. anti-CD20 (such as rituximab), anti-CD22 (such as epratuzumab), anti-CD23 (such as lumiliximab) or anti-CD52 (such as alemtuzumab), that recognize antigens on the cell surface. Newer generation antibodies have been generated to the above cell surface antigens. In some embodiments, the modulator is a B cell receptor complex modulator, e.g., anti-CD₂₀, which recognizes a component of the B cell receptor co-complex, or a B cell receptor activator such as a cross-linker of the B cell receptor complex or the B-cell co-receptor complex. In some embodiments, the cross-linker is an antibody, or molecular binding entity. In some embodiments, the cross-linker is an antibody, such as a multivalent antibody. In some embodiments, the antibody is a monovalent, bivalent, or multivalent antibody made more multivalent by attachment to a solid surface or tethered on a nanoparticle surface to increase the local valency of the epitope binding domain. In some embodiments, the cross-linker is a molecular binding entity, such as an entity that acts upon or binds the B cell receptor complex via carbohydrates or an epitope in the complex. In some embodiments, the molecular binding entity is a monovalent, bivalent, or multivalent binding entity that is made more multivalent by attachment to a solid surface or tethered on a nanoparticle surface to increase the local valency of the epitope binding domain. In some embodiments where the modulator is a B cell receptor modulator, e.g., a B cell receptor activator such as a cross-linker of the B cell receptor complex or the B-cell co-receptor complex, cross-linking includes binding of an antibody or molecular binding entity to the cell and then causing its crosslinking via interaction of the cell with a solid surface that causes crosslinking of the BCR complex via antibody or molecular binding entity. In some embodiments, the crosslinker is selected from the group consisting of F(ab)2 IgM, IgG, IgD, polyclonal BCR antibodies, monoclonal BCR antibodies, Fc receptor derived binding elements and/or a combination thereof. In some embodiments, the Ig is derived from a species selected from the group consisting of mouse, goat, rabbit, pig, rat, horse, cow, shark, chicken, or llama. In some embodiments, the crosslinker is F(ab)2 IgM, Polyclonal IgM antibodies, Monoclonal IgM antibodies, Biotinylated F(ab)2 IgG/M, Biotinylated Polyclonal IgM antibodies, Biotinylated Monoclonal IgM antibodies and/or a combination thereof.

In some embodiments of the methods of the invention, the cell is further subjected to a second modulator, e.g., the cell may be subjected to a B cell receptor activator and a kinase inhibitor Such as a PI3 kinase inhibitor or a JAK inhibitor (see U.S. Ser. Nos. 61/226,878 and 61/157,900 which are hereby incorporated by reference) or a phosphatase inhibitor. In some embodiments, the second modulator is F(ab)₂IgM or F(ab)₂IgM and H₂0₂.

In some embodiments, the modulator is PMA, BAFF, April, SDF1a, SCF, CD40L, IGF-1, Imiquimod, polyCpG, fludarabine, cyclophosphamide, chlorambucil IL-7, IL-6, IL-10, IL-27, IL-4, IL-2, IL-3, thapsigargin and/or a combination thereof.

In some embodiments, the activatable element is a protein. In some embodiments, the protein is selected from the group consisting of Akt1, Akt2, Akt3, SAPK/JNK1,2,3, p38s, Erk1/2, Syk, ZAP-70, Btk, BLNK, Lyn, PLCγ, PLC?γ2, STAT1, STAT3, STAT4, STAT5, STATE, CREB, Lyn, p-S6, Cbl, NF-κB, GSK3β, CARMA/Bcl10 and Tcl-1. In some embodiments, the activatable element is STAT5, PLCγ, Syk, Erk, or Lyn. In some embodiments, these markers are used to predict response to fludarabine.

In some embodiments, tonic signaling (ligand independent signaling) is shown in a subset of CLL patients by using H₂0₂ alone or in combination with a crosslinker, such as F(ab)2IgM. In some embodiments, if the cell demonstrates evidence of tonic signaling after treatment with H₂0₂, then that is one embodiment of a predictive response to a drug, such as fludarabine as one example.

In one embodiment of the invention, tonic signaling is shown by measuring canonical B cell signaling molecules such as p-Lyn, p-Syk, p-BLNK, p-PLCγ2, p-Erk, p-Akt, p-S6, p-65/RelA, as well as non-canonical signaling markers such as p-STAT5.

In some embodiments, ZVAD is used as a modulator to analyze cell death pathways to investigate whether a therapeutic agent affects caspase independent or caspase dependent pathways. ZVAD will block caspase dependant cleavage and it can be used to distinguish caspase-dependent from caspase-independent cell death. This analysis is useful to determine if test substances or drugs will affect either apoptotic pathway and whether both caspase-dependent and caspase-independent pathways are necessary for a therapeutic agent to effectively promote cell death.

In another embodiment, mixture models are used to assess response to treatment. A sample signaling profile may be compared to a standard signaling profile and a result determined. In one embodiment, data generated from the tests described herein are compared to a standard profile defined by a mixture model derived from measurements from one or a plurality of samples. Data can be used to create a profile of results for patients in order to predict who will respond to a particular therapeutic regimen, those who will not, and variations thereof. Test results may be compared to a standard profile once it is created and correlations to responses may be derived. A test may be structured so that an individual patient sample may be viewed with these populations in mind and allocated to one population or the other, or a mixture of both and subsequently to use this correlation to patient management, therapy, prognosis, etc.

In another aspect, the invention provides methods of classifying a cell population by contacting the cell population with at least one modulator, where the modulator is F(ab)2 IgM, an anti-CD20 antibody (such as rituximab), anti-CD52 antibody (such as alemtuzumab), anti-CD22 antibody(such as epratuzumab), anti-CD23 antibody (such as lumiliximab), bendamustine, velcade, phenylarsine oxide, sodium vanadate, H₂O₂, PMA, BAFF, April, SDF1a, CD40L, IGF-1, Imiquimod, polyCpG, fludarabine, cyclophosphamide, chlorambucil, IL-7, IL-6, IL-10, IL-27, IL-4, IL-2, IL-3, thapsigargin and/or a combination thereof, determining the presence or absence of an increase in activation level of an activatable element in the cell population, and classifying the cell population based on the presence or absence of the increase in the activation of the activatable element.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows Basal phosphorylation levels of B cell receptor signaling molecules. Intracellular phosphoflow cytometry was used to measure basal levels of phosphorylation of signaling molecules downstream of the BCR in gated B cells from peripheral blood mononuclear cells taken from CLL or healthy donors. Comparison between CLL B cells (left) and healthy B cells (right) showed greater variability in the B cells from the patient group with the exception of p-Erk and p-65 (p-values from Student's t-test comparing Arcsinh transformed MFI values from CLL and healthy B cells shown on right).

FIG. 2 shows H₂O₂ treatment segregates CLL samples into two groups based on their magnitude of BCR-mediated signaling. (A) CLL B cells were left untreated or stimulated for 10 minutes with anti-μ or anti-γ (10 μg/ml) alone (labeled as BCR X-link), H₂O₂ (3.3 mM) alone or the combination. Cells were fixed and permeabilized before they were incubated at 4° C. overnight with the core gating antibodies supplemented different antibody panels (Table 2). Two dimensional contour plots show exemplary samples in which CLL B cell subsets exhibit robust signaling mediated by H₂O₂ alone and some additional changes with the addition of a BCR cross-linking agent (for sample CLL014) for proximal BCR signaling molecules. Of note are distinct cell subsets with different signaling capacities within each sample. (B) Two dimensional contour plots show exemplary samples in which CLL B cell subsets show a reduced response to H₂O₂ alone as determined for proximal BCR signaling molecules. (C) STAT5, although not part of the canonical BCR network demonstrates either an increase in phosphorylation in response to H₂O₂ alone (left-hand columns) or a marginal response (right-hand columns). The two dimensional plot has SHP-2 along the X-axis as the SHP-2 antibody was in the same antibody panel as the p-STAT5 antibody. Samples CLL014 and CLL024 show distinct cell subsets with different p-STAT5 signaling capacities.

FIG. 3 shows In vitro exposure of CLL B cells to F-ara-A. Cells were exposed to vehicle or F-ara-A (1 μM) for 48 hours at 37° C. Cells were harvested and incubated with an antibody panel comprising the gating antibodies and antibodies recognizing components of the apoptotic cascade (Table 2). The two dimensional contour plots (cleaved caspase 3 (X-axis) and cleaved PARP (Y-axis)) show that samples CLL014 and CLL024 undergo apoptosis (left-hand panels, double positive for cleaved PARP and Caspase-3, left arrows) in response to F-ara-A treatment. Notably, in these samples there were also cell subsets which were refractory to F-ara-A.

FIG. 4 shows histograms comparing population distributions of all CLL and all healthy B cells based on their fluorescence intensities. (A) Arcsinh transformed fluorescence intensities from all gated CLL and healthy B cells were used to derive the histograms. CLL samples demonstrate multiple examples of bimodal activation, as revealed by modulated signaling (dashed lines) after phosphatase inhibition. See samples with arrows, third column. By contrast healthy B cells demonstrate a single cell subset (solid lines) with minimal activation of signaling. (B) Mixture models were generated from the histograms (dashed lines) of arcsinh transformed fluorescence intensities of the CLL B cells comprised of two normal distributions using the mixdistpackage (Efroni S, Schaefer C F, Buetow K H (2007) Identification of Key Processes Underlying Cancer Phenotypes Using Biologic Pathway Analysis. PLoS ONE 2(5): e425); http://icarus.math.mcmaster.ca/peter/mix/mixdist.pdf for R (http://www.r-project.org). To determine component cell populations in a given sample, metrics were defined by computing the area under the curve for the fluorescent intensities of all cells from that sample with respect to a random sampling of 50000 events representing each mixture model-derived distribution. These metrics were termed ‘MixMod1’ and ‘MixMod2’ representing the areas under the curve for the distributions with lower (solid lines) and higher (short-dashed lines) mean fluorescent intensities, respectively. Two normal probability density populations of CLL cells that have a high and low response to signaling molecules downstream of the BCR are depicted by the arrows in the third column. mediated signaling: Signaling heterogeneity observed in outlier cells.

FIG. 5 shows association between H₂O₂-mediated signaling and apoptosis induction by F-ara-A. (A) Area under the receiver operating characteristic (AUROC) curves were expressed with 95% confidence limits in order to evaluate how statistically significant H₂O₂-induced signaling is in predicting an in vitro apoptotic response to F-ara-A. The mixture model metric for H₂O₂-mediated signaling was used to calculate whether there was an association with response or lack of response to in vitro exposure to F-ara-A. A value of 0.5 for the ROC plots indicates that the association is due to chance. A value of 1.0 indicates that there is a perfect association. (B) Example of a Mixture Model showing H₂O₂-mediated increase in p-STAT5 and its ability to predict response to F-ara-A for an individual patient. An unscaled mixture model was derived from the mixture model for H₂O₂-mediated p-STAT5 signaling (top panel and FIG. 4B). Samples CLL007 and CLL021 have one population distribution of cells and are refractory to F-ara-A exposure. Samples CLL014 and CLL024 show population distributions of cells that span both subpopulations. CLL B cells in these samples are responsive to F-Ara-A exposure. CLL009 has a signaling profile predictive of apoptotic sensitivity but was refractory to in vitro F-ara-A. This latter sample does not fit the model presumably due to alternative pathways that confer refractoriness to apoptosis. Short-dashed line on the lower part of the scale-population density distribution defined by MixMod1, heavy-dashed line on the higher part of the scale-population density distribution defined by MixMod2, solid line-population density distribution for H₂O₂-mediated p-STAT5 for B cells from an individual patient.

FIG. 6 shows statistical association between H₂O₂-mediated signaling and apoptosis induction by F-ara-A (Fludarabine) in the group comprised of all CLL cells regardless of ZAP-70 or IgV_{H} mutational status compared with the group comprised of ZAP-70 positive or IgV_{H} unmutated status. (A) ROC curves from a fold change model were expressed in order to evaluate how statistically significant H₂O₂-induced signaling is in predicting an in vitro apoptotic response to F-ara-A for all CLL cells, regardless of ZAP-70 or IgV_{H} mutational status (that is, prediction of apoptotic response is based on H₂O₂-induced nodes). The fold change metric for H₂O₂-mediated signaling was used to calculate whether there was an association with response or lack of response to in vitro exposure to F-ara-A. A value of 0.5 for the ROC plots indicates that the association is due to chance. A value of 1.0 indicates that there is a perfect association. (B) ROC curves from a fold change model were expressed with 95% confidence limits to evaluate how statistically significant H₂O₂-induced signaling is in predicting in vitro apoptotic response to F-ara-A for cells with ZAP-70 positive or IgV_{H} unmutated status (that is, prediction of apoptotic response is based on H₂O₂-induced nodes in combination with ZAP-70 or IgVH status).

### DETAILED DESCRIPTION OF THE INVENTION

Objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the invention will become apparent to those skilled in the art from this detailed description.

All publications, patents, and patent applications mentioned in this specification are herein incorporated by reference to the same extent as if each individual publication or patent application was specifically and individually indicated to be incorporated by reference

This application incorporates by reference, in their entireties, U.S. Ser. No. 60/957,160 filed Aug. 21, 2007, U.S. Ser. No. 61/048,920 filed Apr. 29, 2008 and U.S. Ser. No. 12/229,476 filed Aug. 21, 2008.

The present invention incorporates information disclosed in other applications and texts. The following patent and other publications are hereby incorporated by reference in their entireties: Haskell et al, Cancer Treatment, 5^{th} Ed., W. B. Saunders and Co., 2001; Alberts et al., The Cell, 4^{th} Ed., Garland Science, 2002; Vogelstein and Kinzler, The Genetic Basis of Human Cancer, 2d Ed., McGraw Hill, 2002; Michael, Biochemical Pathways, John Wiley and Sons, 1999; Weinberg, The Biology of Cancer, 2007; Immunobiology, Janeway et al. 7^{th} Ed., Garland, and Leroith and Bondy, Growth Factors and Cytokines in Health and Disease, A Multi Volume Treatise, Volumes 1A and 1B, Growth Factors, 1996. Patents and applications that are also incorporated by reference include U.S. Pat. Nos. 7,381,535 and 7,393,656 and U.S. Ser. Nos. 10/193,462; 11/655,785; 11/655,789; 11/655,821; 11/338,957, 12/460,029, 61/048,886; 61/048,920; 61/048,657; 61/079,766; 61/155,362; 61/079,579; 61/079,537; 61/079,551; 61/087,555 and 61/085,789. See especially, U.S. Ser. No. 12/229,476 including the figures. Some commercial reagents, protocols, software and instruments that are useful in some embodiments of the present invention are available at the Becton Dickinson Website http://www.bdbiosciences.com/features/products/, and the Beckman Coulter website, http://www.beckmancoulter.com/Default.asp?bhfv=7. Relevant articles include High-content single-cell drug screening with phosphospecific flow cytometry, Krutzik et al., Nature Chemical Biology, 23 December (2007); Irish et al., FLt3 ligand Y591 duplication and Bcl-2 over expression are detected in acute myeloid leukemia cells with high levels of phosphorylated wild-type p53, Neoplasia, (2007), Irish et al. Mapping normal and cancer cell signaling networks: towards single-cell proteomics, Nature (2006) 6:146-155; and Irish et al., Single cell profiling of potentiated phospho-protein networks in cancer cells, Cell, (2004) 118, 1-20; Schulz, K. R., et al., Single-cell phospho-protein analysis by flow cytometry, Curr Protoc Immunol, (2007) 78:8 8.17.1-20; Krutzik, P. O., et al., Coordinate analysis of murine immune cell surface markers and intracellular phosphoproteins by flow cytometry, J. Immunol. (2005) 175(4):2357-65; Krutzik, P. O., et al., Characterization of the murine immunological signaling network with phosphospecific flow cytometry, J Immunol. (2005) 175(4):2366-73; Shulz et al., Current Protocols in Immunology (2007) 78:8.17.1-20; Stelzer et al. Use of Multiparameter Flow Cytometry and Immunophenotyping for the Diagnosis and Classification of Acute Myeloid Leukemia, Immunophenotyping, Wiley, 2000; and Krutzik, P.O. and Nolan, G. P., Intracellular phospho-protein staining techniques for flow cytometry: monitoring single cell signaling events, Cytometry A. (2003) 55(2):61-70; Hanahan D., Weinberg, The Hallmarks of Cancer, CELL (2000) 100:57-70; Krutzik et al, High content single cell drug screening with phosphospecific flow cytometry, Nat Chem Biol. (2008) 4:132-42; and Monroe, J. G., Ligand independent tonic signaling in B-cell receptor function, Current Opinion in Immunology 2004, 16:288-295. Experimental and process protocols and other helpful information can be found at http:/proteomices.stanford.edu. The articles and other references cited below are also incorporated by reference in their entireties for all purposes.

### Introduction

In some embodiments, this invention is directed to methods and compositions for diagnosis, prognosis and to methods of treatment. In some embodiments, the physiological status of cells present in a sample (e.g. clinical sample) is used, e.g., in diagnosis or prognosis of a condition, patient selection for therapy, to monitor treatment, modify therapeutic regimens, and to further optimize the selection of therapeutic agents; which may be administered as one or a combination of agents. Hence, therapeutic regimens can be individualized and tailored according to the data obtained prior to, and at different times over the course of treatment, thereby providing a regimen that is individually appropriate.

In some embodiments, the present invention is directed to methods for classifying a sample derived from an individual having or suspected of having a condition, e.g., a neoplastic, autoimmune or a hematopoietic condition. The invention allows for identification of prognostically and therapeutically relevant subgroups of conditions and prediction of the clinical course of an individual. The methods of the invention provide tools useful in the treatment of an individual afflicted with a condition, including but not limited to methods of choosing a therapy for an individual, methods of predicting response to a therapy for an individual, methods of determining the efficacy of a therapy in an individual, methods for assigning a risk group, methods of predicting an increased risk of relapse, methods of predicting an increased risk of developing secondary complications, and methods of determining the prognosis for an individual. The present invention provides methods that can serve as a prognostic indicator to predict the course of a condition, e.g. whether the course of a neoplastic, autoimmune or a hematopoietic condition in an individual will be aggressive or indolent, thereby aiding the clinician in managing the patient and evaluating the modality of treatment to be used.

In some embodiments, the invention is directed to methods for determining the activation level of one or more activatable elements in a cell upon treatment with one or more modulators. The activation of an activatable element in the cell upon treatment with one or more modulators can reveal operative pathways in a condition that can then be used, e.g., choose a therapy for an individual, predict response to a therapy for an individual, determine the efficacy of a therapy in an individual. In some embodiments the modulators may themselves be used directly within individuals as therapeutic agents. In some embodiments the activation of an activatable agent may be used as an indicator to predict course of the condition, identify risk group, predict an increased risk of developing secondary complications, and determine the prognosis for an individual.

In some embodiments, the invention is directed to methods for classifying a cell by contacting the cell with an inhibitor, determining the presence or absence of an increase in activation level of an activatable element in the cell, and classifying the cell based on the presence or absence of the increase in the activation of the activatable element. In some embodiments, the invention is directed to methods of determining the presence or absence of a condition in an individual by subjecting a cell from the individual to a modulator and an inhibitor, determining the activation level of an activatable element in the cell, and determining the presence or absence of the condition based on the activation level upon treatment with a modulator and an inhibitor.

In some embodiments, the invention is directed to methods for classifying a cell by contacting the cell with an inhibitor, determining the presence or absence of a change in activation level of an activatable element in the cell, and classifying the cell based on the presence or absence of the change in the activation of the activatable element. In some embodiments the change is an increase. In some embodiments the change is a decrease.

In some embodiments, the invention is directed to methods of determining tonic signaling status of a cell by subjecting the cell to a modulator, determining the activation level of an activatable element that participates in a tonic signaling pathway in the cell, and determining the status of a tonic signaling pathway in the cell from the activation level. Tonic signaling in a cell may have functional consequences, for instance, to maintain certain differentiated cellular properties or functions. In some embodiments of the invention, the status of a tonic signaling pathway is used to correlate the status to differences in populations.

In some embodiments, the invention is directed to methods of determining a phenotypic profile of a population of cells by exposing the population of cells, optionally in separate cultures, to a plurality of modulators, wherein at least one of the modulators is an inhibitor, determining the presence or absence of an increase in activation level of an activatable element in the cell population from each of the separate culture and classifying the cell population based on the presence or absence of the increase in the activation of the activatable element from populations of cells in each separate culture.

In some embodiments a method for classifying a cell comprises contacting the cell with an inhibitor, determining the presence or absence of a change in an activation level of at least one activatable element in said cell, and classifying said cell based on said presence or absence of said change in the activation level of said at least one activatable element. In some embodiments the change is an increase. In some embodiments the change is a decrease.

In some embodiments the method of classifying a cell further comprises determining the level of an intracellular marker, cell surface marker or any combination thereof. For example a cell may be classified by a change in activation level of an activatable element and also by the level of one or more cell surface markers. In addition a cell may be classified by a change in activation level of an activatable element and by the level of an intracellular marker. Combinations may also be used. Serum markers are also useful in methods of diagnosis, prognosis, determining treatments effects and/or choosing a treatment.

One or more cell surface markers may also be used in the method of the invention in addition to intracellular markers (e.g. phospho-proteins). In some embodiments, the method comprises determining the level of a plurality of cell surface markers. Cell surface markers may include any cell surface molecule that is detected by flow cytometry. In some embodiments the cell surface marker is a human leukocyte differentiation antigen. In some embodiments the human leukocyte differentiation antigen is selected from the list: CD1, CD2, CD3, CD4, CD5, CD8, CD10, CD14, CD19, CD20, CD22, CD23, CD40, CD52, CD100, CD280, CD281, CD282, CD283, CD284, and CD289. In some embodiments the human leukocyte differentiation antigen is selected from the list comprising CD1 though CD300. In some embodiments the cell surface marker is any cell surface receptor or ligand. Examples of cell surface ligands and receptors include, but are not limited to, members of the TNF superfamily, interleukins, hormones, neurotransmitters, interferons, growth factors, chemokines, integrins, toll receptor ligands, prostaglandins, or leukotriene families. Other examples of cell surface markers include, but are not limited to metalloproteases. In some embodiments the cell surface marker is membrane bound IgM. In some embodiments the cell surface marker is a B-cell receptor (BCR) or a component of a BCR. In some embodiments the marker is CD45, CD5, CD14, CD19, CD20, CD22, CD23, CD27, CD37, CD40, CD52, CD79, CD38, CD96, major histocompatability antigen (MHC) Class 1 or MHC Class 2. In some embodiments the cell surface marker is membrane bound IgD. In some embodiments the cell surface marker is membrane bound IgG. In some embodiments, the method of classifying a cell comprises determining a level of at least one cell surface marker on said cell and an activation level of at least one activatable element on said cell. In some embodiments, the method of classifying a cell comprises determining the level of cell surface IgM on said cell. In some embodiments, the method comprises determining the level of cell surface IgD on said cell. In some embodiments, the method comprises determining the level of a BCR on said cell. In some embodiment the cell surface marker is associated with a disease or conditions. In some embodiments the maker is CD38 or CD96. In some embodiments the marker is CD38 and the condition is leukemia. In some embodiments the marker is CD96 and the condition is leukemia.

One or more intracellular markers may be used in the method of the invention. The levels of these markers can be determined before they are secreted and are referred to as “captured”. Examples of captured intracellular markers include, but are not limited to, TNF superfamily members, interleukins, hormones, neurotransmitters, interferons, growth factors, chemokines, integrins, prostaglandins, leukotrines and receptors for all of the above. Examples of intracellular markers also include, but are not limited to, metalloproteases. Examples of intracellular markers also include, but are not limited to, proteins involved in programmed cell death and proliferation. Examples of intracellular markers also include, but are not limited to viruses, pathogens, parasites and components or products thereof. In some embodiments, the method of classifying a cell further comprises determining the level of an intracellular pathogen or component of a pathogen. In some embodiments the intracellular pathogen is HIV. In some embodiments the intracellular pathogen is EBV. In some embodiments the intracellular component of a pathogen is a nucleic acid sequence derived from said pathogen. In some embodiments the intracellular component of a pathogen is a pathogen derived polypeptide.

The method of the invention may comprise determining the level of one or more serum markers. In some embodiments the serum marker is a marker of a condition. In some embodiments the serum marker is a marker of inflammation. In some embodiments the serum marker is a soluble cytokine, TNF superfamily member, interleukin, hormone, neurotransmitter, interferon, growth factor, chemokine, integrin, prostaglandin, leukotriene or any soluble receptor thereof. In some embodiments the serum marker is a marker of a specific disease or condition. In some embodiments the serum marker is a cancer marker. In some embodiments the serum marker is a leukemia marker. In some embodiments the serum marker is beta-2-microglobulin, calcitonin, CD20, CD23, CD52, IL6, IL2R, ICAM-1, CD14, IgG, thymidine kinase or ferritin. In some embodiments the serum marker is a pharmaceutical drug, pathogen, virus, parasite, small compound or toxin. Therefore, in some embodiments, the methods described herein are for diagnosis, prognosis or determining a method of treatment for a subject or patient. In some embodiments the methods comprise classifying a cell or population of cells. In certain embodiments, the methods of diagnosis, prognosis or determining a method of treatment comprise determining the level of at least one serum marker derived from the subject or patient. In some embodiments the serum marker is a cytokine, chemokine, soluble receptor, growth factor, antibody or binding protein. In some embodiments the serum marker is a pathogen. In some embodiments the serum marker is a pharmaceutical compound or drug.

In one embodiment, the present invention can distinguish between responders and non-responder cells from patients after those cells are treated with an anti-cancer agent, such as 9-β-D-arabinosyl-2-fluoroadenine (F-ara-A), the free nucleoside of fludarabine. In an embodiment of the invention, CLL cells are contacted with modulators, such as F(ab)2 IgM (also called anti-g) and H₂0₂ alone or combined together. Activatable elements such as phosphorylated Lyn, Syk, PLCγ2, BLNK, STAT5, Erk, p65/RelA, Akt (Akt1, Akt2, Akt3), S6, Chk2, cleaved PARP, cleaved caspase 3, cleaved caspase 8, cytosolic cytochrome C and Bcl-2 expression are analyzed to assist in the correlation between responses in cells and clinical outcomes.

The subject invention also provides kits for use in determining the physiological status of cells in a sample, the kit comprising one or more specific binding elements for signaling molecules, and may additionally comprise one or more therapeutic agents. The kit may further comprise a software package for data analysis of the physiological status, which may include reference profiles for comparison with the test profile.

As disclosed herein is a method for classifying a cell comprising contacting the cell with a modulator or an inhibitor used to determine the presence or absence of a change in activation level of an activatable element in the cell, and classifying the cell based on the presence or absence of the change in the activation level of the activatable element. In some embodiments the change in activation level of an activatable element is an increase in the activation level of an activatable element. In some embodiments the activatable element is a protein subject to phosphorylation or dephosphorylation.

In some embodiments, one aspect of the invention is tyrosine phosphatase inhibitor (e.g. peroxide) mediated STAT5 or AKT phosphorylation to segregate or stratify patients. In another embodiment, the invention relates to measuring in vitro apoptosis in response to F-ara-A into separate classes of patients who are apoptosis competent or refractory. Another aspect of the invention relates to the use of classification and modeling methods such as logistic regression (including regularized, penalized, and shrinkage methods including lasso and ridge), decision trees, random forests, support vector machines, boosting, etc. to generate univariate and multivariate models associating tyrosine phosphatase inhibitor (e.g. hydrogen peroxide (H₂0₂)) or B-cell receptor cross linking induced changes in phosphorylation with the ability of cells to undergo apoptosis. Another aspect of the invention is the detection of ZAP-70 to increase the predictability of the area under the ROC curve or the use of the ROC curve to determine the suitability of a classification and modeling method. Another aspect of the invention relates to the use of mixture models to represent data for the uses disclosed herein. In another embodiment, detection of ZAP-70, IGVH and/or CD38 can be used as clinical covariates that can be combined with phosphorylation and/or signaling readouts, in multivariate models of the methods described throughout the specification.

In some embodiments of the methods, the invention provides a method for classifying a cell by contacting the cell with an inhibitor; determining the activation levels of a plurality of activatable elements in the cell; and classifying the cell based on the activation level. In some embodiments, the inhibitor is a kinase or phosphatase inhibitor, such as adaphostin, AG 490, AG 825, AG 957, AG 1024, aloisine, aloisine A, alsterpaullone, aminogenistein, API-2, apigenin, arctigenin, AY-22989, BAY 61-3606, bisindolylmaleimide IX, chelerythrine, 10-[4′-(N,N-Diethylamino)butyl]-2-chlorophenoxazine hydrochloride, dasatinib, 2-Dimethylamino-4,5,6,7-tetrabromo-1H-benzimidazole, 5,7-Dimethoxy-3-(4-pyridinyl)quinoline dihydrochloride, edelfosine, ellagic acid, enzastaurin, ER 27319 maleate, erlotinib, ET18OCH₃, fasudil, flavopiridol, gefitinib, GW 5074, H-7, H-8, H-89, HA-100, HA-1004, HA-1077, HA-1100, hydroxyfasudil, indirubin-3′-oxime, 5-Iodotubercidin, kenpaullone, KN-62, KY12420, LFM-A13, lavendustin A, luteolin, LY-294002, LY294002, mallotoxin, ML-9, NSC-154020, NSC-226080, NSC-231634, NSC-664704, NSC-680410, NU6102, olomoucine, oxindole I, PD-153035, PD-98059, PD-169316, phloretin, phloridzin, piceatannol, picropodophyllin, PK1, PP1, PP2, purvalanol A, quercetin, R406, R788, rapamune, rapamycin, Ro 31-8220, roscovitine, rottlerin, SB202190, SB203580, sirolimus, sorafenib, SL327, SP600125, staurosporine, STI-571, SU-11274, SU1498, SU4312, SU6656, 4,5,6,7-Tetrabromotriazole, TG101348, Triciribine, Tyrphostin AG 490, Tyrphostin AG 825, Tyrphostin AG 957, Tyrphostin AG 1024, Tyrphostin SU1498, U0126, VX-509, VX-667, VX-680, W-7, wortmannin, XL-019, XL-147, XL-184, XL-228, XL-281, XL-518, XL-647, XL-765, XL-820, XL-844, XL-880, Y-27632, ZD-1839, ZM-252868, ZM-447-439, H₂O₂, siRNA, miRNA, Cantharidin, (−)-p-Bromotetramisole, Microcystin LR, Sodium Orthovanadate, Sodium Pervanadate, Vanadyl sulfate, Sodium oxodiperoxo(1,10-phenanthroline)vanadate, bis(maltolato)oxovanadium(IV), Sodium Molybdate, Sodium Perm olybdate, Sodium Tartrate, Imidazole, Sodium Fluoride, β-Glycerophosphate, Sodium Pyrophosphate Decahydrate, Calyculin A, Discodermia calyx, bpV(phen), mpV(pic), DMHV, Cypermethrin, Dephostatin, Okadaic Acid, NIPP-1, N-(9,10-Dioxo-9,10-dihydro-phenanthren-2-yl)-2,2-dimethyl-propionamide, α-Bromo-4-hydroxyacetophenone, 4-Hydroxyphenacyl Br, α-Bromo-4-methoxyacetophenone, 4-Methoxyphenacyl Br, α-Bromo-4-(carboxymethoxy)acetophenone, 4-(Carboxymethoxy)phenacyl Br, and bis(4-Trifluoromethylsulfonamidophenyl)-1,4-diisopropylbenzene, phenyarsine oxide, Pyrrolidine Dithiocarbamate, or Aluminum fluoride. In some embodiments the phosphatase inhibitor is a tyrosine phosphatase inhibitor, such as H₂O₂.

In some embodiments the cell or cell population (hereinafter called a “cell”) is a hematopoietic-derived cell. In some embodiments, the hematopoietically derived cell is selected from the group consisting of pluripotent hematopoietic stem cells, B-lymphocyte lineage progenitor or derived cells, T-lymphocyte lineage progenitor or derived cells, NK cell lineage progenitor or derived cells, granulocyte lineage progenitor or derived cells, monocyte lineage progenitor or derived cells, megakaryocyte lineage progenitor or derived cells and erythroid lineage progenitor or derived cells. In some embodiments, the hematopoietic derived cell is a B-lymphocyte lineage progenitor and derived cell, e.g., an early pro-B cell, late pro-B cell, large pre-B cell, small pre-B cell, immature B cell, mature B cell, plasma cell and memory B cell, a CD5+ B cell, a CD38+ B cell, a B cell bearing a mutated or non mutated heavy chain of the B cell receptor, or a B cell expressing ZAP-70.

In some embodiments, the classification or correlation includes classifying the cell as a cell that is correlated with a clinical outcome. In some embodiments, the clinical outcome is the prognosis and/or diagnosis of a condition. In some embodiments, the clinical outcome is the presence or absence of a neoplastic, autoimmune or a hematopoietic condition, such as Non-Hodgkin Lymphoma, Hodgkin or other lymphomas, acute or chronic leukemias, polycythemias, thrombocythemias, multiple myeloma or plasma cell disorders, e.g., amyloidosis and Waldenstrom's macroglobulinemia, myelodysplastic disorders, myeloproliferative disorders, myelofibrosis, or atypical immune lymphoproliferations, systemic lupus erythematosis (SLE), rheumatoid arthritis (RA). In some embodiments, the neoplastic, autoimmune or hematopoietic condition is non-B lineage derived, such as acute myeloid leukemia (AML), Chronic Myeloid Leukemia (CML), non-B cell acute lymphocytic leukemia (ALL), non-B cell lymphomas, myelodysplastic disorders, myeloproliferative disorders, myelofibrosis, thrombocythemias, or non-B atypical immune lymphoproliferations. In some embodiments, the neoplastic, autoimmune or hematopoietic condition is a B-Cell or B cell lineage derived disorder, such as Chronic Lymphocytic Leukemia (CLL), B-cell lymphoma, B lymphocyte lineage leukemia, B lymphocyte lineage lymphoma, Multiple Myeloma, acute lymphoblastic leukemia (ALL), B-cell pro-lymphocytic leukemia, precursor B lymphoblastic leukemia, hairy cell leukemia or plasma cell disorders, e.g., amyloidosis or Waldenstrom's macroglobulinemia, B cell lymphomas including but not limited to diffuse large B cell lymphoma, follicular lymphoma, mucosa associated lymphatic tissue lymphoma, small cell lymphocytic lymphoma and mantle cell lymphoma. In some embodiments, the condition is CLL. In some embodiments, the CLL is defined by a monoclonal B cell population that co-expresses CD5 with CD19 and CD23 or CD5 with CD20 and CD23 and by surface immunoglobulin expression.

In some embodiments, the clinical outcome is the staging or grading of a neoplastic, autoimmune or hematopoietic condition. Examples of staging in methods provided by the invention include aggressive, indolent, benign, refractory, Roman Numeral staging, TNM Staging, Rai staging, Binet staging, WHO classification, FAB classification, IPSS score, WPSS score, limited stage, extensive stage, staging according to cellular markers such as ZAP-70 and CD38, occult, including information that may inform on time to progression, progression free survival, overall survival, or event-free survival.

In some embodiments of the invention, the activation level of the plurality of activatable elements in the cell is selected from the group consisting of cleavage by extracellular or intracellular protease exposure, novel hetero-oligomer formation, glycosylation level, phosphorylation level, acetylation level, methylation level, biotinylation level, glutamylation level, glycylation level, hydroxylation level, isomerization level, prenylation level, myristoylation level, lipoylation level, phosphopantetheinylation level, sulfation level, ISGylation level, nitrosylation level, palmitoylation level, SUMOylation level, ubiquitination level, neddylation level, citrullination level, deamidation level, disulfide bond formation level, proteolytic cleavage level, translocation level, changes in protein turnover, multi-protein complex level, oxidation level, multi-lipid complex, and biochemical changes in cell membrane. In some embodiments, the activation level is a phosphorylation level. In some embodiments, the activatable element is selected from the group consisting of proteins, carbohydrates, lipids, nucleic acids and metabolites. In some embodiments, the activatable element is a protein. In some embodiments, the activatable element is a change in metabolic state, temperature, or local ion concentration. In embodiments where the activatable element is a protein, in some embodiments the protein is a protein subject to phosphorylation or dephosphorylation, such as kinases, phosphatases, adaptor/scaffold proteins, ubiquitination enzymes, adhesion molecules, contractile proteins, cytoskeletal proteins, heterotrimeric G proteins, small molecular weight GTPases, guanine nucleotide exchange factors, GTPase activating proteins, caspases and proteins involved in apoptosis (e.g. PARP), ion channels, molecular transporters, molecular chaperones, metabolic enzymes, vesicular transport proteins, hydroxylases, isomerases, transferases, deacetylases, methylases, demethylases, proteases, esterases, hydrolases, DNA binding proteins or transcription factors. In some embodiments, the protein is selected from the group consisting of PI3-Kinase (p85, p110a, p110b, p110d), Jak1, Jak2, SOCs, Rac, Rho, Cdc42, Ras-GAP, Vav, Tiam, Sos, Dbl, Nck, Gab, PRK, SHP1, and SHP2, SHIP1, SHIP2, sSHIP, PTEN, Shc, Grb2, PDK1, SGK, Akt1, Akt2, Akt3, TSC1,2, Rheb, mTor, 4EBP-1, p70S6Kinase, S6, LKB-1, AMPK, PFK, Acetyl-CoAa Carboxylase, DokS, Rafs, Mos, Tp12, MEK1/2, MLK3, TAK, DLK, MKK3/6, MEKK1,4, MLK3, ASK1, MKK4/7, SAPK/JNK1,2,3, p38s, Erk1/2, Syk, Btk, BLNK, LAT, ZAP-70, Lyn, Cbl, SLP-76, PLCγ₁, PLCγ2, transcription factor, STAT1, STAT3, STAT4, STAT5, STATE, FAK, p130CAS, PAKs, LIMK1/2, Hsp90, Hsp70, Hsp27, SMADs, Rel-A (p65-NFκB), CREB, Histone H₂B, HATs, HDACs, PKR, Rb, Cyclin D, Cyclin E, Cyclin A, Cyclin B, P16, p14Arf, p27KIP, p21CIP, Cdk4, Cdk6, Cdk7, Cdk1, Cdk2, Cdk9, Cdc25, A/B/C, Abl, E2F, FADD, TRADD, TRAF2, RIP, Myd88, BAD, Bcl-2, Mcl-1, Bcl-XL, Caspase 2, Caspase 3, Caspase 6, Caspase 7, Caspase 8, Caspase 9, PARP, IAPB, Smac, Fodrin, Actin, Src, Lyn, Fyn, Lyn, NIK, IκB, p65(RelA), IKKα, PKA, PKCα, PKCβ, PKCθ, PKCδ, CAMK, Elk, AFT, Myc, Egr-1, NFAT, ATF-2, Mdm2, p53, DNA-PK, Chk1, Chk2, ATM, ATR, \tilde{β}catenin, CrkL, GSK3a, GSK3β, and FOXO. In some embodiments, the protein selected from the group consisting of Erk, Syk, ZAP-70, Lyn, Btk, BLNK, Cbl, PLCγ2, Akt, RelA, p38, S6. In some embodiments the protein is S6.

In some embodiments, the protein is selected from the group consisting of HER receptors, PDGF receptors, Kit receptor, FGF receptors, Eph receptors, Trk receptors, IGF receptors, Insulin receptor, Met receptor, Ret, VEGF receptors, TIE1, TIE2, FAK, Jak1, Jak2, Jak3, Tyk2, Src, Lyn, Fyn, Lyn, Fgr, Yes, Csk, Abl, Btk, ZAP-70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Weel, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3a, GSK3β, Cdks, CLKs, PKR, PI3-Kinase class 1, class 2, class 3, mTor, SAPK/JNK1,2,3, p38s, PKR, DNA-PK, ATM, ATR, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, SHIPs, myotubularins, phosphoinositide kinases, phospholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, IL-2, IL-4, IL-8, IL-6, interferon γ, interferon α, suppressors of cytokine signaling (SOCs), Cbl, SCF ubiquitination ligase complex, APC/C, adhesion molecules, integrins, Immunoglobulin-like adhesion molecules, selectins, cadherins, catenins, focal adhesion kinase, p130CAS, fodrin, actin, paxillin, myosin, myosin binding proteins, tubulin, eg5/KSP, CENPs, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, Vav, Tiam, Sos, Dbl, PRK, TSC1,2, Ras-GAP, Arf-GAPs, Rho-GAPs, caspases, Caspase 2, Caspase 3, Caspase 6, Caspase 7, Caspase 8, Caspase 9, PARP, Bcl-2, Mcl-1, Bcl-XL, Bcl-w, Bcl-B, A1, Bax, Bak, Bok, Bik, Bad, Bid, Bim, Bmf, Hrk, Noxa, Puma, IAPB, XIAP, Smac, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, Pin1 prolyl isomerase, topoisomerases, deacetylases, Histone deacetylases, sirtuins, histone acetylases, CBP/P300 family, MYST family, ATF2, DNA methyl transferases, Histone H3K4 demethylases, H3K27, JHDM2A, UTX, VHL, WT-1, p53, Hdm, PTEN, ubiquitin proteases, urokinase-type plasminogen activator (uPA) and uPA receptor (uPAR) system, cathepsins, metalloproteinases, esterases, hydrolases, separase, potassium channels, sodium channels, multi-drug resistance proteins, P-Glycoprotein, nucleoside transporters, Ets, Elk, SMADs, Rel-A (p65-NFKB), CREB, NFAT, ATF-2, AFT, Myc, Fos, Sp1, Egr-1, T-bet, β-catenin, HIFs, FOXOs, E2Fs, SRFs, TCFs, Egr-1, β-catenin, FOXO transcription factor, STAT1, STAT2, STAT3, STAT4, STAT5a, STAT5b, STATE, p53, WT-1, HMGA, pS6, 4EPB-1, eIF4E-binding protein, RNA polymerase, initiation factors, elongation factors.

In some embodiments of the methods of the invention, the modulator to which the cell is subjected is an activator or an inhibitor. In some embodiments, the modulator is, e.g., a growth factor, cytokine, adhesion molecule modulator, hormone, small molecule, polynucleotide, antibodies, natural compounds, lactones, chemotherapeutic agents, immune modulator, carbohydrate, proteases, ions, reactive oxygen species, or radiation. In some embodiments, the modulator is a B cell receptor modulator, e.g., a B cell receptor activator such as a cross-linker of the B cell receptor complex or the B-cell co-receptor complex. In some embodiments of the invention, the cell is subjected to a modulator and a separate B cell receptor modulator (such as a B cell receptor cross-linker). In some embodiments, the cross-linker is an antibody, or molecular binding entity. In some embodiments, the cross-linker is an antibody, such as a multivalent antibody. In some embodiments, the antibody is a monovalent, bivalent, or multivalent antibody made more multivalent by attachment to a solid surface or tethered on a nanoparticle surface to increase the local valency of the epitope binding domain. In some embodiments, the cross-linker is a molecular binding entity, such as an entity that acts upon or binds the B cell receptor complex via carbohydrates or an epitope in the complex. In some embodiments, the molecular binding entity is a monovalent, bivalent, or multivalent binding entity that is made more multivalent by attachment to a solid surface or tethered on a nanoparticle surface to increase the local valency of the epitope binding domain. In some embodiments where the modulator is a B cell receptor modulator, e.g., a B cell receptor activator such as a cross-linker of the B cell receptor complex or the B-cell co-receptor complex, cross-linking includes binding of an antibody or molecular binding entity to the cell and then causing its crosslinking via interaction of the cell with a solid surface that causes crosslinking of the BCR complex via antibody or molecular binding entity. In some embodiments, the crosslinker is selected from the group consisting of F(ab)2 IgM, IgG, IgD, polyclonal BCR antibodies, monoclonal BCR antibodies, and Fc receptor derived binding elements. The Ig may be derived from a species selected from the group consisting of mouse, goat, rabbit, pig, rat, horse, cow, shark, chicken, or llama. In some embodiments, the crosslinker is F(ab)2 IgM, Polyclonal IgM antibodies, Monoclonal IgM antibodies, Biotinylated F(ab)2 IgG/M, Biotinylated Polyclonal IgM antibodies, Biotinylated Monoclonal IgM antibodies and/or a combination thereof.

In some embodiments of the methods of the invention, the cell is subjected to a B cell receptor activator and a phosphatase inhibitor or kinase inhibitor, such as F(ab)₂IgM or biotinylated F(ab)₂IgM and a phosphatase inhibitor (e.g., H₂0₂).

In some embodiments, the invention provides a method of determining a tonic signaling (ligand independent) status of a cell by subjecting the cell to a modulator, determining the activation level of an activatable element that participates in a tonic signaling pathway in the cell, and determining the status of a tonic signaling pathway in the cell from the activation level. In some embodiments, a condition of an individual is determined based on tonic signaling status of a cell. In some embodiments, the condition is a neoplastic, autoimmune and/or hematopoietic condition as discussed above.

In some embodiments, the tonic signaling status of a cell is correlated with a clinical outcome such as prognosis or diagnosis of the condition.

In some embodiments, the correlation is determining the individual's response to a treatment, e.g., normal responder, hyper responder, poor responder, having emerging resistance, non-compliant, and adverse reaction.

In some embodiments of this aspect, the invention provides a method of correlating an activation level of a B-lymphocyte lineage derived cell with a neoplastic, autoimmune or hematopoietic condition in an individual by subjecting the B-lymphocyte lineage derived cell from the individual to a modulator; determining the activation levels of a plurality of activatable elements that participate in a tonic signaling pathway in the B-lymphocyte lineage derived cell; and identifying a pattern of the activation levels of the plurality of activatable elements in the tonic signaling pathway in the cell that correlates with a clinical outcome, such as the prediction of outcome for a particular treatment, a prognosis or diagnosis of a certain condition (e.g., a neoplastic condition).

In some embodiments of the methods of the invention, the cell is further subjected to a second modulator, e.g., the cell may be subjected to a B cell receptor activator and a phosphatase inhibitor, such as F(ab)₂IgM or biotinylated F(ab)₂IgM and a phosphatase inhibitor (e.g., H₂0₂).

In addition to determining the activation level of an activatable protein, in some embodiments the methods for classifying a cell further comprise determining the level of an additional intracellular marker and/or a cell surface marker. In some embodiments the methods for classifying a cell comprise determining the level of an additional intracellular marker. In some embodiments the intracellular marker is a captured intracellular cytokine. In some embodiments the methods for classifying a cell comprise determining the level of an additional cell surface marker. In some embodiments the cell surface marker is a cell surface ligand or receptor. In some embodiments the cell surface marker is a component of a B-cell receptor complex. In some embodiments the cell surface marker is CD45, CD5, CD19, CD20, CD22, CD23, CD27, CD37, CD40, CD52, CD79, CD38, CD96, major histocompatability antigen (MHC) Class 1 or MHC Class 2.

In some embodiments the methods of the invention for prognosis, diagnosis, or determination of treatment further comprise determining the level of an additional serum marker. In some embodiments the serum marker comprises a protein. In some embodiments the serum marker is a cytokine, growth factor, chemokine, soluble receptor, small compound, or pharmaceutical drug. In some embodiments the serum marker comprises a component or product of a pathogen or parasite. In some embodiments the serum marker is selected from a group consisting of beta-2-microglobulin, calcitonin, thymidine kinase and ferritin.

In some embodiments, the invention provides a method of correlating an activation level of B-lymphocyte lineage derived cells with a neoplastic, autoimmune or hematopoietic condition in an individual by subjecting the B-lymphocyte lineage derived cell from the individual to a modulator; determining the activation levels of a plurality of activatable elements in the B-lymphocyte lineage derived cell; and identifying a pattern of the activation levels of the plurality of activatable elements in the cell that correlates with the neoplastic condition. In some embodiments, the activatable element is selected from the group consisting of elements selected from the group consisting of Erk, Syk, ZAP-70, Lyn, Btk, BLNK, Cbl, PLCγ2, Akt, RelA, p38, S6 (which can be phosphorylated). In some embodiments, the activatable element is selected from the group consisting of Cbl, PLCγ2, and S6. In some embodiments, the activatable element is S6. In some embodiments, the B-lymphocyte lineage progenitor or derived cell is selected from the group consisting of early pro-B cell, late pro-B cell, large pre-B cell, small pre-B cell, immature B cell, mature B cell, plasma cell and memory B cell, a CD5+ B cell, a CD38+ B cell, a B cell bearing a mutilated or non mutated heavy chain of the B cell receptor, or a B cell expressing ZAP-70. In some embodiments, the invention provides methods for correlating and/or classifying an activation state of a CLL cell with a clinical outcome in an individual by subjecting the CLL cell from the individual to a modulator, where the CLL cell expresses a B-Cell receptor (BCR), determining the activation levels of a plurality of activatable elements, and identifying a pattern of the activation levels of the plurality of activatable elements to determine the presence or absence of an alteration in signaling proximal to the BCR, wherein the presence of the alteration is indicative of a clinical outcome.

In some embodiments the method comprises identifying a pattern of said activation levels of said plurality of activatable elements in said cell, wherein said pattern is correlated to a disease or condition.

In some embodiments, the correlation is determining the individual's response to a specific treatment, e.g., normal responder, hyper responder, poor responder, having emerging resistance, non-compliant, and adverse reaction.

In some embodiments of the invention, the modulator to which the cell is subjected is an activator or an inhibitor. In some embodiments, the modulator is, e.g., a growth factor, cytokine, adhesion molecule modulator, hormone, small molecule, polynucleotide, antibody, natural compound, lactone, chemotherapeutic agent, immune modulator, carbohydrate, protease, ion, reactive oxygen species, or radiation. In some embodiments, the modulator is an antibody, e.g. anti-CD20 (such as rituximab), anti-CD22 (such as epratuzumab), anti-CD23 (such as lumiliximab) or anti-CD52 (such as alemtuzumab), that recognize antigens on the cell surface. Newer generation antibodies have been generated to the above cell surface antigens. In some embodiments, the modulator is a B cell receptor complex modulator, e.g., anti-CD20, which recognizes a component of the B cell receptor co-complex, or a B cell receptor activator such as a cross-linker of the B cell receptor complex or the B-cell co-receptor complex. In some embodiments, the cross-linker is an antibody, or molecular binding entity. In some embodiments, the cross-linker is an antibody, such as a multivalent antibody. In some embodiments, the antibody is a monovalent, bivalent, or multivalent antibody made more multivalent by attachment to a solid surface or tethered on a nanoparticle surface to increase the local valency of the epitope binding domain. In some embodiments, the cross-linker is a molecular binding entity, such as an entity that acts upon or binds the B cell receptor complex via carbohydrates or an epitope in the complex. In some embodiments, the molecular binding entity is a monovalent, bivalent, or multivalent binding entity that is made more multivalent by attachment to a solid surface or tethered on a nanoparticle surface to increase the local valency of the epitope binding domain. In some embodiments where the modulator is a B cell receptor modulator, e.g., a B cell receptor activator such as a cross-linker of the B cell receptor complex or the B-cell co-receptor complex, cross-linking includes binding of an antibody or molecular binding entity to the cell and then causing its crosslinking via interaction of the cell with a solid surface that causes crosslinking of the BCR complex via antibody or molecular binding entity. In some embodiments, the crosslinker is selected from the group consisting of F(ab)2 IgM, IgG, IgD, polyclonal BCR antibodies, monoclonal BCR antibodies, Fc receptor derived binding elements and/or a combination thereof. In some embodiments, the Ig is derived from a species selected from the group consisting of mouse, goat, rabbit, pig, rat, horse, cow, shark, chicken, or llama. In some embodiments, the crosslinker is F(ab)2 IgM, Polyclonal IgM antibodies, Monoclonal IgM antibodies, Biotinylated F(ab)₂ IgG/M, Biotinylated Polyclonal IgM antibodies, Biotinylated Monoclonal IgM antibodies and/or a combination thereof.

In some embodiments of the methods of the invention, the cell is further subjected to a second modulator, e.g., the cell may be subjected to a B cell receptor activator and a kinase inhibitor Such as a PI3 kinase inhibitor or a JAK inhibitor (see U.S. Ser. Nos. 61/226,878 and 61/157,900 which are hereby incorporated by reference) or a phosphatase inhibitor. In some embodiments, the second modulator is F(ab)₂IgM or F(ab)₂IgM and H₂O₂.

In some embodiments, the modulator is PMA, BAFF, April, SDF1a, SCF, CD40L, IGF-1, Imiquimod, polyCpG, fludarabine, cyclophosphamide, chlorambucil IL-7, IL-6, IL-10, IL-27, IL-4, IL-2, IL-3, thapsigargin and/or a combination thereof.

In some embodiments, the activatable element is a protein. In some embodiments, the protein is selected from the group consisting of Akt1, Akt2, Akt3, SAPK/JNK1,2,3, p38s, Erk1/2, Syk, ZAP-70, Btk, BLNK, Lyn, PLCγ, PLC?γ2, STAT1, STAT3, STAT4, STAT5, STATE, CREB, Lyn, p-S6, Cbl, NF-κB, GSK313, CARMA/Bcl10 and Tcl-1. In some embodiments, the activatable element is STAT5, PLCγ, Syk, Erk, or Lyn. In some embodiments, these markers are used to predict response to fludarabine.

In some embodiments, tonic signaling (ligand independent signaling) is shown in a subset of CLL patients by using H₂O₂ alone or in combination with a crosslinker, such as F(ab)₂IgM. In some embodiments, if the cell demonstrates evidence of tonic signaling after treatment with H₂O₂, then that is one embodiment of a predictive response to a drug, such as fludarabine as one example.

In one embodiment of the invention, tonic signaling is shown by measuring canonical B cell signaling molecules such as p-Lyn, p-Syk, p-BLNK, p-PLCγ2, p-Erk, p-Akt, p-S6, p-65/RelA, as well as non-canonical signaling markers such as p-STAT5.

In some embodiments, ZVAD is used as a modulator to analyze cell death pathways to investigate whether a therapeutic agent affects caspase independent or caspase dependent pathways. ZVAD will block caspase dependant cleavage and it can be used to distinguish caspase-dependent from caspase-independent cell death. This analysis is useful to determine if test substances or drugs will affect either apoptotic pathway and whether both caspase-dependent and caspase-independent pathways are necessary for a therapeutic agent to effectively promote cell death.

In another embodiment, mixture models are used to assess response to treatment. A sample signaling profile may be compared to a standard signaling profile and a result determined. In one embodiment, data generated from the tests described herein are compared to a standard profile defined by a mixture model derived from measurements from one or a plurality of samples. Data can be used to create a profile of results for patients in order to predict who will respond to a particular therapeutic regimen, those who will not, and variations thereof. Test results may be compared to a standard profile once it is created and correlations to responses may be derived. A test may be structured so that an individual patient sample may be viewed with these populations in mind and allocated to one population or the other, or a mixture of both and subsequently to use this correlation to patient management, therapy, prognosis, etc.

In another aspect, the invention provides methods of classifying a cell population by contacting the cell population with at least one modulator, where the modulator is F(ab)2 IgM, an anti-CD20 antibody (such as rituximab), anti-CD52 antibody (such as alemtuzumab), anti-CD22 antibody(such as epratuzumab), anti-CD23 antibody (such as lumiliximab), bendamustine, velcade, phenylarsine oxide, sodium vanadate, H₂O₂, PMA, BAFF, April, SDF1a, CD40L, IGF-1, Imiquimod, polyCpG, fludarabine, cyclophosphamide, chlorambucil, IL-7, IL-6, IL-10, IL-27, IL-4, IL-2, IL-3, thapsigargin and/or a combination thereof, determining the presence or absence of an increase in activation level of an activatable element in the cell population, and classifying the cell population based on the presence or absence of the increase in the activation of the activatable element.

### CLL

Chronic lymphocytic leukemia (CLL) is the most common adult leukemia in the Western world and is characterized by aberrant accumulation of CD5+ B lymphocytes in the peripheral blood, bone marrow and secondary lymphoid organs. Clinical presentation, natural course of the disease and response to treatment are all extremely variable with survival ranging from months to decades. Although the biological mechanisms to account for this unpredictable clinical course are unknown, several biological indicators have been linked to CLL. However, there is often discordance between their predictive value for disease outcome. Thus predisposing factors in determining the clinical benefit of these markers include whether the disease is at early or late stage as well as the treatment that the patient may have undergone. A variety of cytogenetic abnormalities including del(17p13.1), del(11q22.3), trisomy 12 are associated with poor prognosis while del(13q.1.4.3) is associated with a more favorable clinical course (Hallck 2008, Hamblin 2007 Ghia et al., 2007). In addition, at least two predominant subtypes of CLL have been identified based on the presence or absence of somatic mutations within the immunoglobulin heavy chain variable region (IgV_{H}). The outcome of patients with leukemic cells that express unmutated. IgV_{H} is poor compared to that of patients in which leukemic cells express mutated IgV_{H} ((Hamblin 1999), Ghia et al, Crit. Rev. Oncol. Hematol. 2007, 2008). The latter is commonly but not invariably linked to positive expression of ZAP-70 and CD38 (Damle 1999). Moreover, considerable molecular heterogeneity between leukemic samples has been identified through gene array technology (Rodriguez et al 2007). Given the availability of newly approved and investigational therapeutic agents, a greater understanding of CLL disease biology is needed to predict disease progression and assist in selecting optimal therapeutic agents on an individual patient basis.

Studies in leukemia have described a new approach in which single cell network profiling in response to extracellular inputs (such as growth factors and cytokines) can be used to distinguish healthy from diseased cells (Irish Cell (2004), Irish Blood (2006), Irish et al., Nat Rev Cancer (2006), Kotecha et al., (2008)). Induced protein phosphorylation rather than the frequently measured basal phosphorylation state of a protein is more informative as it takes into account signaling dysregulation that is the consequence of numerous cytogenetic, epigenetic and molecular changes characteristic of transformed cells. Multiparameter flow cytometry at the single cell level not only measures multiple phospho-signaling proteins but also delineates cell sub-sets within complex primary cell populations. No prior sorting of cell subpopulations is required before challenge with an extracellular modulator.

Central to B cell development is the role of the B cell receptor signal complex composed of a surface immunoglobulin molecule non-covalently associated with the signal transducing-CD79α/CD79β heterodimer. In normal B cells, stimulation of the BCR by antigen leads to phosphorylation of immunoreceptor tyrosine-based activation motifs (ITAMs) within the cytoplasmic tails of CD79α and CD79β. Subsequent recruitment of Syk to these motifs propagates a signal through activation of downstream signaling molecules such as BLNK, phosphatidylinositol-3-Kinase, phospholipase C-γ and the Ras/Raf/Erk pathways (Brerski and Monroe 2008, Efremov et al., Autoimmunity Reviews 2007 Kurosaki et al., J. Exp Med 182, p1815, 1995, Takata et al., J. Exp Med 184, p31 1996). During normal B cell maturation, signals from the B cell receptor lead to remarkably different biochemical responses depending on the developmental stage in which the β-cell resides (Gauld et al). Additionally, there is essential fine tuning of BCR signaling for the survival and proliferation of healthy B cells, which has been shown to involve phosphatase(s) whose activity is regulated by NADPH-oxidase-generated H₂O₂ (Reth M. Nat. Immunol. 2002; 3:1129-1134 and Irish J M, J. Immunol. 2006; 177:1581-1589).

Recently, it has been recognized that in conjunction with antigen-driven responses, ligand-independent signaling, (termed tonic signaling) by the pre-B cell receptor and BCR has an important role in B cell development and in mature B cells respectively. In addition to the recognized role of CD79α and CD79β, tyrosine phosphatases are also likely to impact on tonic signaling. This is based on a study of B cells in which inhibition of these phosphatases by H₂0₂ or vanadate revealed tyrosine phosphorylation of signaling proteins associated with the BCR (Wienands, J., Larbolette, O. & Roth, M.PNAS (1996), Reth Nat Rev. Immunol 2002, Monroe (2006) Irish et al., J. Immunol. 2006).

As mentioned above, significant associations have been observed between clinical course of CLL and certain features of the BCR, indicating that antigen-dependent and independent stimulation and signaling may play an important role in the pathogenesis of the disease Most CLL B cells express a BCR comprised of IgM with or without somatic mutations. In addition, the biased usage of V_{H} genes, the preferential usage of kappa or lambda light-chains, as well as aberrant expression of BCR signaling mediators suggest a central role for the B receptor signaling network in CLL. This is corroborated by in vitro studies in which significant differences in BCR signaling were found in CLL primary patient samples (Chen et al. Blood 2007, Gobessi Blood 2007 Deglesne et al., Can Res 66, p7158, 2006, Muzio et al., Blood 2008 112, p188 Guarini et al, Blood 2008 112 p782).

Under normal physiological conditions, apoptosis proceeds from sensors that monitor cell stress and damage to effectors that relay the signals to activate programmed cell death pathways. In cancer, cells have co-opted a variety of mechanisms to evade apoptosis for the purpose of survival and disease progression and also to over-ride any benefit from a therapeutic agent (Hanahan and Wienberg, Cell 2000) CLL is no different in that it may show inactivated p53 signaling (17p deletion) in a subset of patients. In other patient subsets, different mechanisms over-riding apoptosis have evolved resulting in refractoriness or resistance to therapies. The current study was undertaken to determine whether and how ligand dependent and ligand independent (tonic) BCR signaling was associated with subverted apoptotic pathways in patient samples exposed in vitro to fludarabine, a drug at the core of many CLL treatment regimens.

Based on prior studies in leukemic samples, modulated SCNP using flow cytometry was applied to determine whether there were subsets of cells between samples and within the same sample that showed: a) alterations in BCR responses b) differences in fludarabine-induced apoptosis c) associations between BCR signaling and in vitro chemosensitivity to fludarabine. The data presented here suggest tonic BCR signaling may play a role in the response mediated by agents that induce apoptosis.

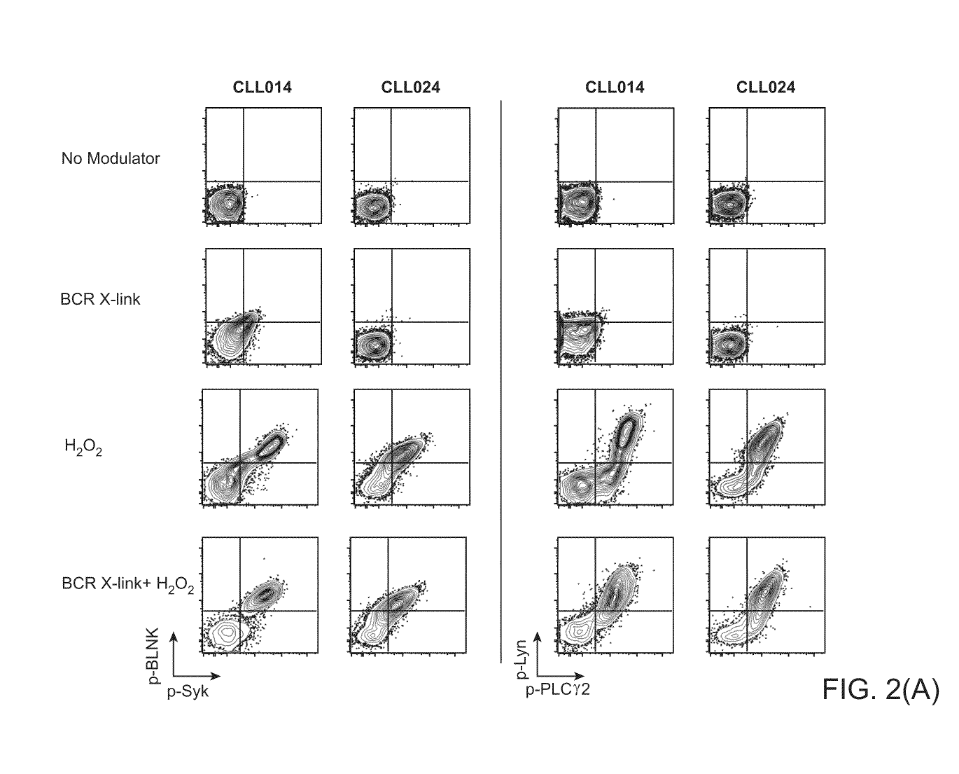

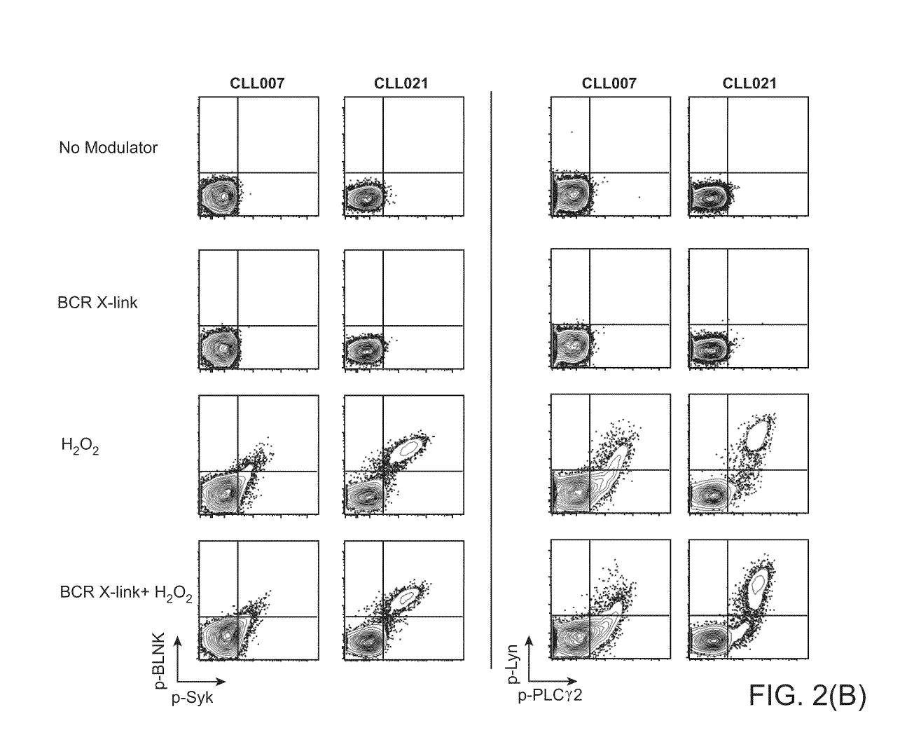

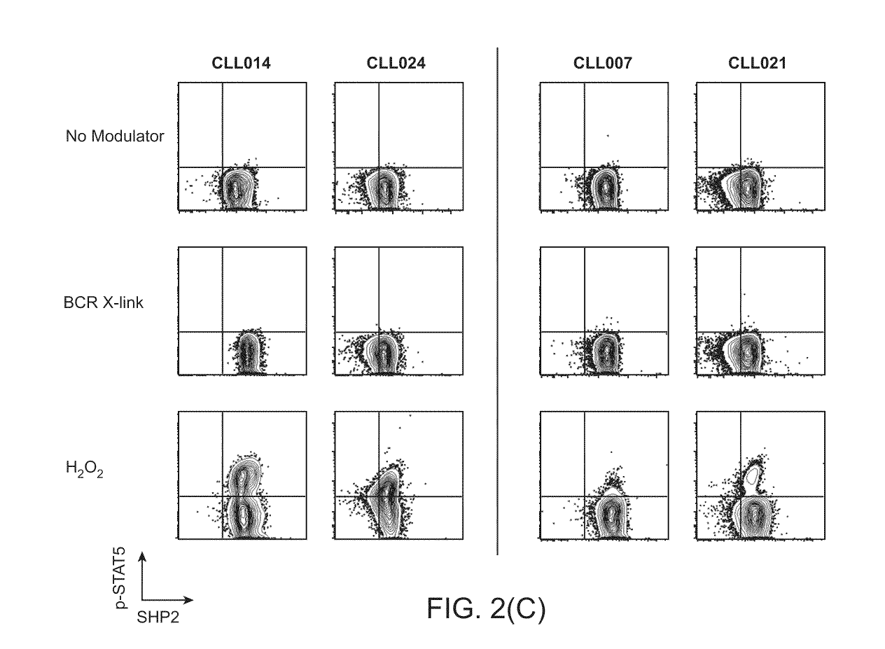

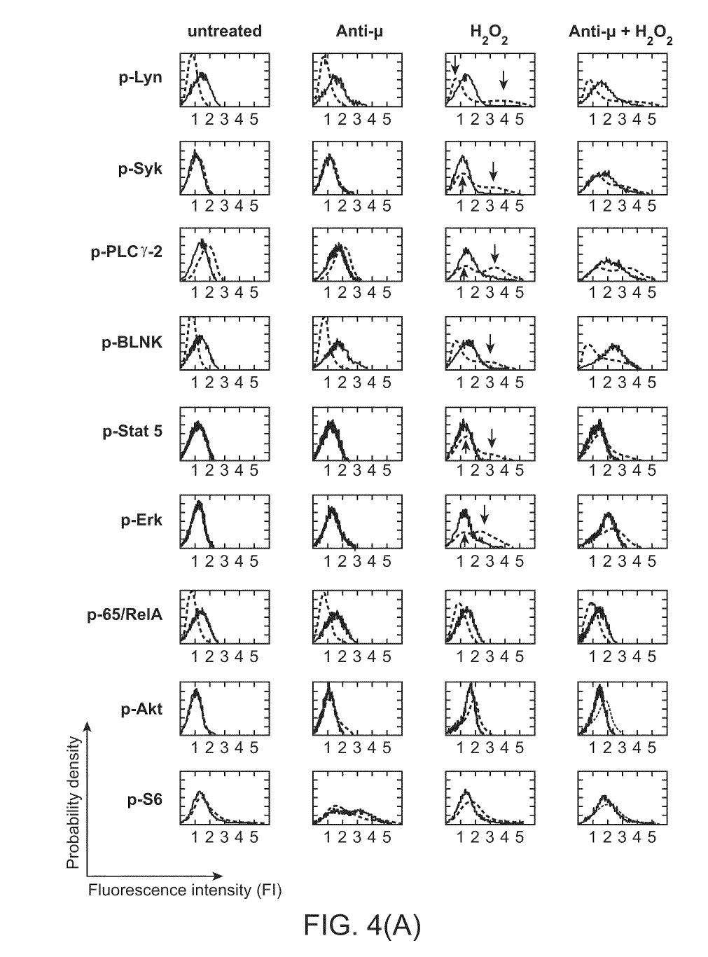

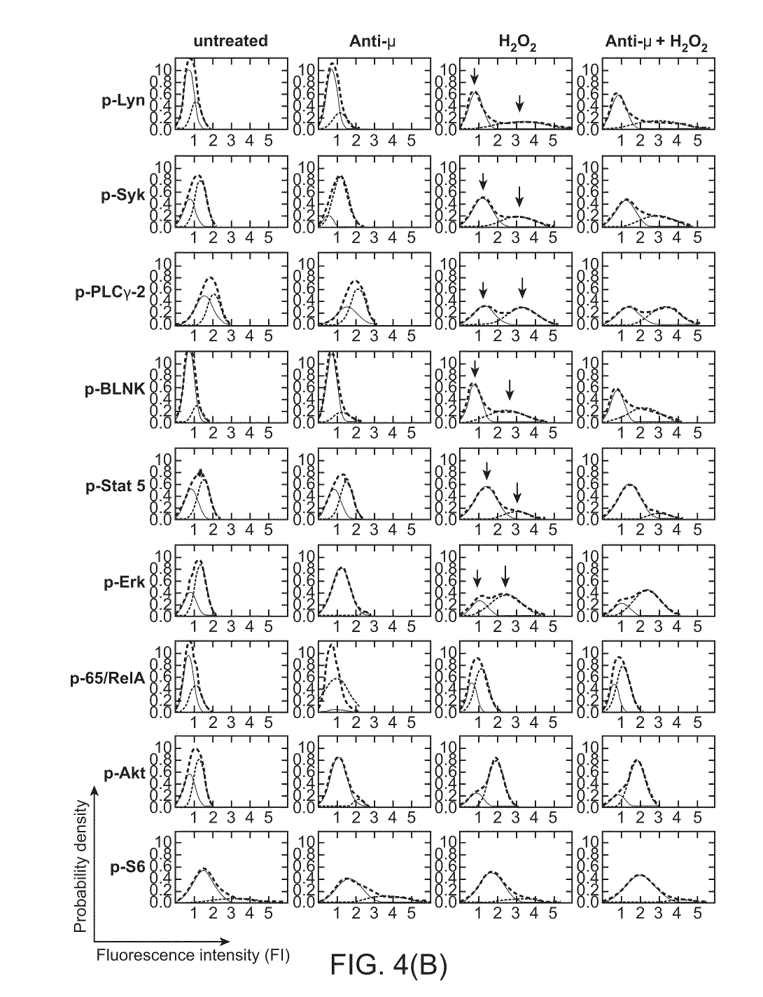

In one aspect of the invention, the range of basal signaling in CLL B cells from patient samples is very broad compared to B cells from healthy donors. Mixture models show the distribution of different signaling subpopulations within a sample. A mixture model is created by making a virtual sample by looking at the distribution of signaling subpopulations in an entire cohort of samples. (FIGS. 4A, B). Heterogeneity of signaling is seen within cell subsets in individual samples. The heterogeneity is revealed by treatment of the sample with a modulator including, but not limited to H₂0₂. Such heterogeneity is not observed by monitoring the basal phosphorylation state in the absence of a modulator. This heterogeneity could have therapeutic implications. One or more cell subsets in a sample with a differential signaling response could be, for example a therapeutically resistant clone. See FIG. 2A patient sample CLL014 treated with H₂0₂.

In some CLL samples, subpopulations of B cells undergo an increase in p-STAT5 in response to modulators, including but not limited to phosphatase inhibitors such as H₂O₂. See FIG. 2C.

Another embodiment of the invention is detection of B cell subsets within CLL patient samples (that are refractory or competent) to undergo apoptosis induced by in vitro treatment of therapeutic agents including, but not limited to fludarabine. This drug forms the core of many CLL patient treatment regimens.

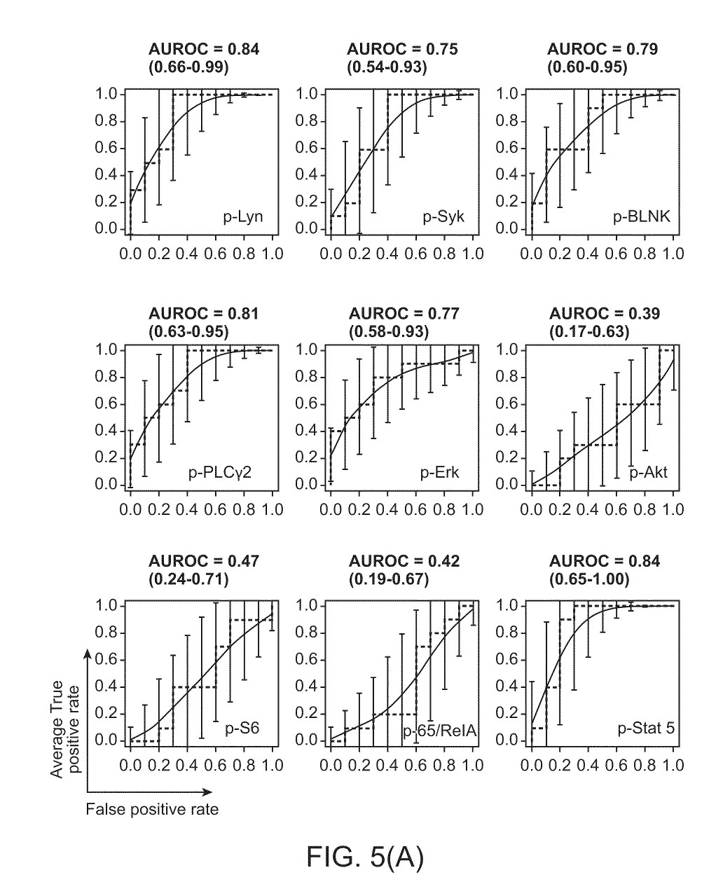

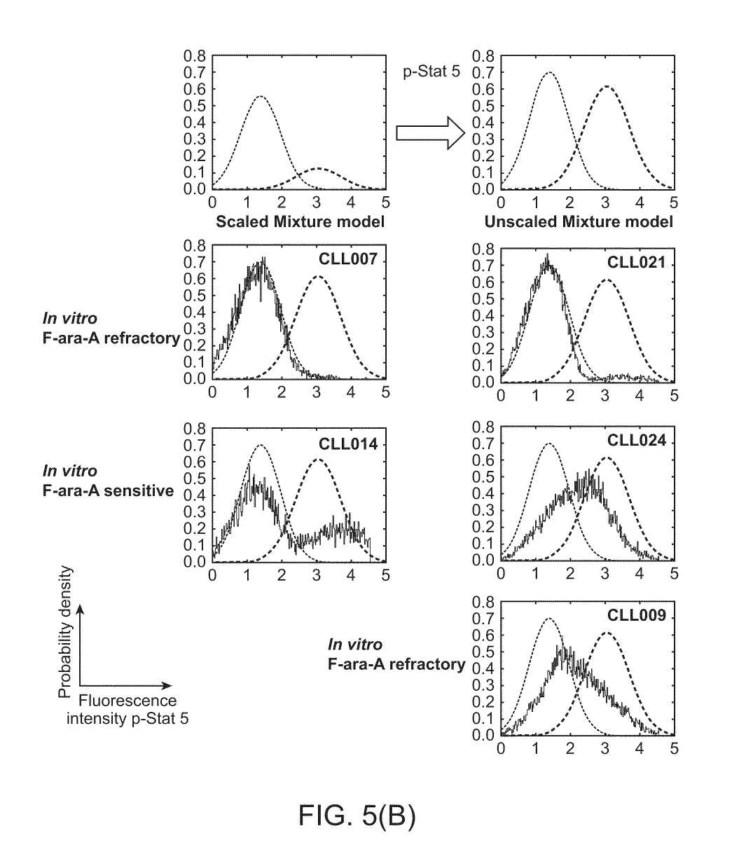

Patients can be stratified by the modulated signaling responses in their CLL samples. Patients can also be stratified by the apoptotic response of their CLL samples exposed in vitro to therapeutic agents such as fludarabine. Apoptotic responses stratify the patient samples into those that are competent versus those that are refractory. Also, the level of signaling stratifies patient samples during basal and/or modulated signaling states. The present data shows that there is an association between increased signaling responses and an ability to undergo apoptosis. Another embodiment of the invention relates to the statistical methods used to demonstrate these biological pathway associations. These statistical methods include, but are not limited to Area Under the Receiver Operating Characteristic (AUROC) (see FIG. 5A) using metrics including, but not limited to the mixture models shown in FIGS. 4A and 4B. The AUROC curves show that increased phosphorylation of Lyn, Syk, BLNK, PLCγ2, Erk, and STAT5 are highly predictive of cell subpopulations competent to undergo apoptosis in vitro. See FIG. 5A. An AUROC value greater than 0.5 can indicate an improved predictive value as opposed to chance association. An AUROC value of one indicates that the predictive value is perfect. An AUROC which has a value>0.65>0.70>0.75>0.8>0.85>0.9>0.95>0.97 could form the basis for a predictive test for patient management. In some embodiment, the methods of the invention determine the presence or absence of a change in activation level of at least two activatable elements of Lyn, Syk, BLNK, PLCγ2, Erk or STAT5 in a cell.

In some embodiments of the invention, detection using mixture models and univariate or multivariate analysis described above can be used in a predictive test for diagnosis and/or patient management, for example, using classification and modeling methods such as logistic regression (including regularized, penalized, and shrinkage methods including lasso and ridge), decision trees, random forests, support vector machines, boosting, etc. to generate univariate and multivariate models. In some embodiments, analysis can be done using univariate and multivariate models associating hydrogen peroxide (H₂0₂) or B-cell receptor cross linking induced changes in phosphorylation with the ability of cells to undergo apoptosis.

Another embodiment of the invention allows a user to understand whether the signaling data for an intracellular signaling molecule is predictive for the apoptotic response of a sample from an individual patient. As such, it can be the basis of a test to determine whether a patient's CLL disease will respond to a therapeutic agent including, but not limited to fludarabine. See FIG. 5B.

### Methods

In some embodiments, the invention provides methods, including methods to determine the physiological status of a cell, e.g., by determining the activation level of an activatable element upon contact with one or more modulators. In some embodiments, the invention provides methods, including methods to classify a cell according to the status of an activatable element in a cellular pathway. The information can be used in prognosis and diagnosis, including susceptibility to disease(s), status of a diseased state and response to changes, in the environment, such as the passage of time, treatment with drugs or other modalities. The physiological status of the cells provided in a sample (e.g. clinical sample) may be classified according to the activation of cellular pathways of interest. The cells can also be classified as to their ability to respond to therapeutic agents and treatments.

One or more cells, or samples containing one or more cells, can be isolated from body samples, such as, but not limited to, smears, sputum, biopsies, secretions, cerebrospinal fluid, bile, blood, lymph fluid, urine and feces, a lavage of a tissue or organ (e.g. lung) or tissue which has been removed from organs, such as breast, lung, intestine, skin, cervix, prostate, and stomach. For example, a tissue sample can comprise a region of functionally related cells or adjacent cells. Such samples can comprise complex populations of cells, which can be assayed as a population, or separated into sub-populations. Such cellular and acellular samples can be separated by centrifugation, elutriation, density gradient separation, apheresis, affinity selection, panning, FACS, centrifugation with Hypaque, etc. By using antibodies specific for markers identified with particular cell types, a relatively homogeneous population of cells may be obtained. Alternatively, a heterogeneous cell population can be used. Cells can also be separated by using filters. For example, whole blood can also be applied to filters that are engineered to contain pore sizes that select for the desired cell type or class. Rare pathogenic cells can be filtered out of diluted, whole blood following the lysis of red blood cells by using filters with pore sizes between 5 to 10 μm, as disclosed in U.S. patent application Ser. No. 09/790,673. Other devices can separate tumor cells from the bloodstream, see Demirci U, Toner M., Direct etch method for microfluidic channel and nanoheight post-fabrication by picoliter droplets, Applied Physics Letters 2006; 88 (5), 053117; and Irimia D, Geba D, Toner M., Universal microfluidic gradient generator, Analytical Chemistry 2006; 78: 3472-3477. Once a sample is obtained, it can be used directly, frozen, or maintained in appropriate culture medium for short periods of time. Methods to isolate one or more cells for use according to the methods of this invention are performed according to standard techniques and protocols well-established in the art.

Suitable cells include those cell types associated in a wide variety of disease conditions, even while in a non-diseased state. Accordingly, suitable eukaryotic cell types include, but are not limited to, tumor cells of all types (e.g. melanoma, myeloid leukemia, carcinomas of the lung, breast, ovaries, colon, kidney, prostate, pancreas and testes), cardiomyocytes, dendritic cells, endothelial cells, epithelial cells, lymphocytes (T-cell and B cell), mast cells, eosinophils, vascular intimal cells, macrophages, natural killer cells, erythrocytes, hepatocytes, leukocytes including mononuclear leukocytes, stem cells such as hematopoietic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include primary disease state cells, such as primary tumor cells. Suitable cells also include known research cells, including, but not limited to, Jurkat T cells, NIH3T3 cells, CHO, COS, etc. See the ATCC cell line catalog, hereby expressly incorporated by reference.

In some embodiments, the cells are cultured post collection in a media suitable for revealing the activation level of an activatable element (e.g. RPMI, DMEM) in the presence, or absence, of serum such as fetal bovine serum, bovine serum, human serum, porcine serum, horse serum, or goat serum. When serum is present in the media it could be present at a level ranging from 0.0001% to 100%. In some embodiments serum is present in the media at a level ranging from 0.0001% to 90%.

In some embodiments serum is present in the media at a level ranging from 0.01% to 30%. In some embodiments serum is present in the media at 1, 2, 3, 4, 5, 6, 7, 8, 9 or 10%. In some embodiments, serum is present in the media at any suitable level.

In some embodiments, the cell is a hematopoietic cell. Examples of hematopoietic cells include but are not limited to pluripotent hematopoietic stem cells, B-lymphocyte lineage progenitor or derived cells, T-lymphocyte lineage progenitor or derived cells, NK cell lineage progenitor or derived cells, granulocyte lineage progenitor or derived cells, monocyte lineage progenitor or derived cells, megakaryocyte lineage progenitor or derived cells and erythroid lineage progenitor or derived cells.

In some embodiments, the cells used in the present invention are taken from a patient. Cells used in the present invention can be purified from whole blood by any suitable method.

The term “patient” or “individual” as used herein includes humans as well as other mammals. The methods generally involve determining the status of an activatable element. The methods also involve determining the status of a plurality of activatable elements.

In some embodiments, the invention provides a method of classifying a cell by determining the presence or absence of a change in activation level of an activatable element in the cell upon treatment with one or more modulators, and classifying the cell based on the presence or absence of the change in the activation of the activatable element. In some embodiments the change is a decrease. In some embodiments the change is an increase. In some embodiments of the invention, the activation level of the activatable element is determined by contacting the cell with a binding element that is specific for an activation state of the activatable element. In some embodiments, a cell is classified according to the activation level of a plurality of activatable elements after the cell have been subjected to a modulator. In some embodiments of the invention, the activation levels of a plurality of activatable elements are determined by contacting a cell with a plurality of binding element, where each binding element is specific for an activation state of an activatable element.

The classification of a cell according to the status of an activatable element can comprise classifying the cell as a cell that is correlated with a clinical outcome. In some embodiments, the clinical outcome is the prognosis and/or diagnosis of a condition. In some embodiments, the clinical outcome is the presence or absence of a neoplastic, autoimmune or a hematopoietic condition such as those conditions shown in the Summary and under the section marked Conditions.

Modulators include compounds or conditions capable of impacting cellular signaling networks. Modulators can include single or multiple agents. For example, anti-g and H₂O₂ act together in healthy bone marrow cells. A modulator can be an activator or an inhibitor. Modulators can take the form of a wide variety of environmental inputs. Examples of modulators include but are not limited to growth factors, cytokines, chemokines, soluble receptors, Toll-like receptor ligands, pathogens, parasites, components of pathogens or parasites, adhesion molecule modulators, pharmaceutical compounds, drugs, hormones, small molecules, polynucleotides, antibodies, natural compounds, lactones, chemotherapeutic agents, immune modulators, carbohydrates, proteases, ions, reactive oxygen species, radiation, physical parameters such as heat, cold, UV radiation, peptides, and protein fragments, either alone or in the context of cells, cells themselves, viruses, and biological and non-biological complexes (e.g. beads, plates, viral envelopes, antigen presentation molecules such as major histocompatibility complex). Examples of modulators include, but are not limited to, F(ab)2 IgM, rituximab, alemtuzumab, fludarabine, cyclophosphamide, chlorambucil, anti-CD22 antibody (such as epratuzumab), anti-CD23 (such as lumiliximab), Bendamustine, velcade, H₂O₂, PMA, BAFF, April, SDF1a, CD40L, IGF-1, Imiquimod, polyCpG, IL-7, IL-6, IL-10, IL-27, IL-4, IL-2, and IL-3. Additional modulators, inhibitors and activators are disclosed in U.S. 61/085,789 which is hereby incorporated by reference in its entirety. Fludarabine is shown in V. Gandhi and W. Plunkett (2002) Clin. Pharmacokinet 41:93-103, which is hereby incorporated by reference in its entirety.

In some embodiments, the modulator is an activator. In some embodiments the modulator is an inhibitor. In some embodiments, the invention provides methods for classifying a cell by contacting the cell with an inhibitor, determining the presence or absence of a change in activation level of an activatable element in the cell, and classifying the cell based on the presence or absence of the change in the activation of the activatable element. In some embodiments the change is a decrease. In some embodiments the change is an increase. In some embodiments, a cell is classified according to the activation level of a plurality of activatable elements after the cell have been subjected to an inhibitor. In some embodiments, the inhibitor is an inhibitor of a cellular factor or a plurality of factors that participates in a signaling cascade in the cell. In some embodiments, the inhibitor is a kinase or phosphatase inhibitor. Examples of kinase inhibitors include adaphostin, AG 490, AG 825, AG 957, AG 1024, aloisine, aloisine A, alsterpaullone, aminogenistein, API-2, apigenin, arctigenin, AY-22989, BAY 61-3606, bisindolylmaleimide IX, chelerythrine, 10-[4′-(N,N-Diethylamino)butyl]-2-chlorophenoxazine hydrochloride, dasatinib, 2-Dimethylamino-4,5,6,7-tetrabromo-1H-benzimidazole, 5,7-Dimethoxy-3-(4-pyridinyl)quinoline dihydrochloride, edelfosine, ellagic acid, enzastaurin, ER 27319 maleate, erlotinib, ET180CH3, fasudil, flavopiridol, gefitinib, GW 5074, H-7, H-8, H-89, HA-100, HA-1004, HA-1077, HA-1100, hydroxyfasudil, indirubin-3′-oxime, 5-Iodotubercidin, kenpaullone, KN-62, KY12420, LFM-A13, lavendustin A, luteolin, LY-294002, LY294002, mallotoxin, ML-9, NSC-154020, NSC-226080, NSC-231634, NSC-664704, NSC-680410, NU6102, olomoucine, oxindole I, PD-153035, PD-98059, PD 169316, phloretin, phloridzin, piceatannol, picropodophyllin, PK1, PP1, PP2, purvalanol A, quercetin, R406, R788, rapamune, rapamycin, Ro 31-8220, roscovitine, rottlerin, SB202190, SB203580, sirolimus, sorafenib, SL327, SP600125, staurosporine, STI-571, SU-11274, SU1498, SU4312, SU6656, 4,5,6,7-Tetrabromotriazole, TG101348, Triciribine, Tyrphostin AG 490, Tyrphostin AG 825, Tyrphostin AG 957, Tyrphostin AG 1024, Tyrphostin SU1498, U0126, VX-509, VX-667, VX-680, W-7, wortmannin, XL-019, XL-147, XL-184, XL-228, XL-281, XL-518, XL-647, XL-765, XL-820, XL-844, XL-880, Y-27632, ZD-1839, ZM-252868, ZM-447-439, Examples of phosphatase inhibitors include, but are not limited to H₂O₂, siRNA, miRNA, Cantharidin, (−)-p-Bromotetramisole, Microcystin LR, Sodium Orthovanadate, Sodium Pervanadate, Vanadyl sulfate, Sodium oxodiperoxo(1,10-phenanthroline)vanadate, bis(maltolato)oxovanadium(IV), Sodium Molybdate, Sodium Perm olybdate, Sodium Tartrate, Imidazole, Sodium Fluoride, β-Glycerophosphate, Sodium Pyrophosphate Decahydrate, Calyculin A, Discodermia calyx, bpV(phen), mpV(pic), DMHV, Cypermethrin, Dephostatin, Okadaic Acid, NIPP-1, N-(9,10-Dioxo-9,10-dihydro-phenanthren-2-yl)-2,2-dimethyl-propionamide, α-Bromo-4-hydroxyacetophenone, 4-Hydroxyphenacyl Br, α-Bromo-4-methoxyacetophenone, 4-Methoxyphenacyl Br, α-Bromo-4-(carboxymethoxy)acetophenone, 4-(Carboxymethoxy)phenacyl Br, and bis(4-Trifluoromethylsulfonamidophenyl)-1,4-diisopropylbenzene, phenyarsine oxide, Pyrrolidine Dithiocarbamate, and Aluminum fluoride. In some embodiments, the phosphatase inhibitor is H₂O₂.

In some embodiments, the methods of the invention provide methods for determining the presence or absence of a condition in an individual by subjecting a cell from the individual to a modulator and an inhibitor, determining the activation level of an activatable element in the cell, and determining the presence or absence of a condition based on the activation level. In some embodiments, the activation level of a plurality of activatable elements in the cell is determined. The inhibitor can be an inhibitor as described herein. In some embodiments, the inhibitor is a phosphatase inhibitor. In some embodiments, the inhibitor is H₂O₂. The modulator can be any modulator described herein. In some embodiments, the modulator is a B cell receptor modulator. In some embodiments, the B cell receptor modulator is a B cell receptor activator. An example of B cell receptor activator is a cross-linker of the B cell receptor complex or the B-cell co-receptor complex. In some embodiments, cross-linker is an antibody or molecular binding entity. In some embodiments, the cross-linker is an antibody. In some embodiments, the antibody is a multivalent antibody. In some embodiments, the antibody is a monovalent, bivalent, or multivalent antibody made more multivalent by attachment to a solid surface or tethered on a nanoparticle surface to increase the local valency of the epitope binding domain.

The cross-linker can be a molecular binding entity. In some embodiments, the molecular binding entity acts upon or binds the B cell receptor complex via carbohydrates or an epitope in the complex. In some embodiments, the molecular is a monovalent, bivalent, or multivalent is made more multivalent by attachment to a solid surface or tethered on a nanoparticle surface to increase the local valency of the epitope binding domain.

The cross-linking of the B cell receptor complex or the B-cell co-receptor complex can comprise binding of an antibody or molecular binding entity to the cell and then causing its crosslinking via interaction of the cell with a solid surface that causes crosslinking of the BCR complex via antibody or molecular binding entity.

The crosslinker can be F(ab)2 IgM, IgG, IgD, polyclonal BCR antibodies, monoclonal BCR antibodies, Fc receptor derived binding elements and/or a combination thereof. The Ig can be derived from a species selected from the group consisting of mouse, goat, rabbit, pig, rat, horse, cow, shark, chicken, llama or human. The Ig or binding element can be fully human or partially human and can be generated by any suitable method known in the art. In some embodiments, the crosslinker is F(ab)2 IgM, Polyclonal IgM antibodies, Monoclonal IgM antibodies, Biotinylated F(ab)2 IgG/M, Biotinylated Polyclonal IgM antibodies, Biotinylated Monoclonal IgM antibodies and/or a combination thereof.

In some embodiments, the methods of the invention provides for the use of more than one modulator. In some embodiments, the methods of the invention utilize a B cell receptor activator and a phosphatase inhibitor. In some embodiments, the methods of the invention utilize F(ab)₂IgM or biotinylated F(ab)2IgM and H₂0₂.

In some embodiments, the methods of the invention provides for methods of classifying a cell population, or determining a phenotypic profile of a population of cells, by exposing the cell population in separate cultures to a plurality of modulators and determining the status of activatable elements in the cell populations. In some embodiments, the status of a plurality of activatable elements in the cell population, or the phenotypic profile, is determined. In some embodiments, at least one of the modulators of the plurality of modulators is an inhibitor. The modulator can be any modulators described herein. In some embodiments, the modulator is selected from the group consisting of F(ab)2 IgM, H₂O₂, PMA, BAFF, April, SDF1a, CD40L, IGF-1, Imiquimod, polyCpG, IL-7, IL-6, IL-10, IL-27, IL-4, IL-2, IL-3, thapsigargin and a combination thereof. In some embodiments of the invention, the status of an activatable element is determined by contacting the cell population with a binding element that is specific for an activation state of the activatable element. In some embodiments, the status of a plurality of activatable elements is determined by contacting the cell population with a plurality of binding elements, where each binding element is specific for an activation state of an activatable element.

In some embodiments, the methods of the invention provide for methods for classifying a cell population by contacting the cell population with at least one modulator, where the modulator is to F(ab)2 IgM, rituximab, alemtuzumab (such as Campath), anti-CD22 antibody (such as epratuzumab), anti-CD23 antibody (such as lumiliximab), bendamustine, velcade H₂O₂, phenylarsine oxide, sodium vanadate, PMA, BAFF, April, SDF1a, CD40L, IGF-1, Imiquimod, polyCpG, fludarabine, cyclophosphamide, chlorambucil, IL-7, IL-6, IL-10, IL-27, IL-4, IL-2, IL-3, thapsigargin and/or a combination thereof, and determining the status of an activatable element in the cell population. In some embodiments, the status of a plurality of activatable elements in the cell population is determined. In some embodiments of the invention, the status of an activatable element is determined by contacting the cell population with a binding element that is specific for an activation state of the activatable element. In some embodiments, the status of a plurality of activatable elements is determined by contacting the cell population with a plurality of binding elements, where each binding element is specific for an activation state of an activatable element.

In some embodiments, the invention provides a method for classifying a B-lymphocyte progenitor or derived cell as described herein by contacting the cell with a modulator, determining the presence or absence of a change in activation level of an activatable element in the cell, and classifying the cell based on the presence or absence of the change in the activation of the activatable element. In some embodiments the change is a decrease. In some embodiments the change is an increase. In some embodiments, the presence or absence of a change in the activation level of an activatable element is determined by contacting the cell with a binding element that is specific for an activation state of the activatable element. In some embodiments, a B-lymphocyte progenitor or derived cell is classified according to the activation level of a plurality of activatable elements after the cells have been subjected to a modulator. In some embodiments, the presence or absence of a change in the activation levels of a plurality of activatable elements is determined by contacting the cell population with a plurality of binding elements, where each binding elements is specific for an activation state of an activatable element. In some embodiments, the method for classifying a B-lymphocyte progenitor or derived cell further comprises determining the level of at least one cell-surface marker. In some embodiments, the method for classifying a B-lymphocyte progenitor or derived cell further comprises determining the level of at least one intracellular marker, for example a captured intracellular cytokine In some embodiments, the B-lymphocyte progenitor or derived cell is associated with a condition such a neoplastic, autoimmune or hematopoietic condition. Thus, in some embodiments, the invention provides methods for classifying a B-lymphocyte progenitor or derived cell associated with a condition (e.g. neoplastic, autoimmune or hematopoietic condition) by contacting the cell with a modulator, determining the presence or absence of a change in activation level of one or more activatable elements in the cell, and classifying the cell based on the presence or absence of the change in the activation of the one or more activatable elements. In some embodiments the change is a decrease. In some embodiments the change is an increase.

In some embodiments, the invention provides methods for correlating and/or classifying an activation state of a CLL cell with a clinical outcome in an individual by subjecting the CLL cell from the individual to a modulator, wherein the CLL cell expresses B-Cell receptor (BCR), determining the activation levels of a plurality of activatable elements, and identifying a pattern of the activation levels of the plurality of activatable elements to determine the presence or absence of an alteration in signaling proximal to the BCR, wherein the presence of the alteration is indicative of a clinical outcome. In some embodiments, the activation levels of a plurality of activatable elements are determined by contacting the cell with a plurality of binding elements, where each binding element is specific for an activation state of an activatable element. The clinical outcome can be any clinical outcome described herein.

In some embodiments, the methods of the invention provide methods for determining tonic signaling status of a cell by subjecting the cell to a modulator, determining the activation level of an activatable element that participates in a tonic signaling pathway in the cell, and determining the status of a tonic signaling pathway in the cell from the activation level. In some embodiments, the status of a plurality of activatable elements in the cell population is determined. In some embodiments, the activation level of an activatable element is determined by contacting the cell with a binding element that is specific for an activation state of the activatable element. In some embodiments, the activation level of a plurality of activatable elements is determined by contacting the cell with a plurality of binding elements, where each binding element is specific for an activation state of an activatable element. In some embodiments, the tonic signaling is mediated by a cellular receptor. In some embodiments, the tonic signaling is mediated by a T-cell receptor (TCR). In some embodiments, the tonic signaling is mediated by the B-cell receptor (BCR). In some embodiments, the tonic signaling status in the cell is used to classify the cell as described herein.

Patterns and profiles of one or more activatable elements are detected using the methods known in the art including those described herein. In some embodiments, patterns and profiles of activatable elements that are cellular components of a cellular pathway are detected using the methods described herein. In some embodiments, patterns and profiles of activatable elements that are cellular components of a signaling pathway are detected using the methods described herein. In some embodiments, patterns and profiles of activatable elements that are cellular components of a tonic signaling pathway are detected using the methods described herein. For example, patterns and profiles of one or more phosphorylated polypeptide are detected using methods known in art including those described herein.

In some embodiments of the methods described herein, cells (e.g. normal non-transformed cells) other than the cells associated with a condition (e.g. cancer cells) can be used to make clinical decisions. That is that cells, other than cells associated with a condition (e.g. cancer cells), are in fact reflective of the condition process. Normal cells (e.g. healthy cells or non-transformed cells) can be used, e.g., in assigning a risk group, predicting an increased risk of relapse, predicting an increased risk of developing secondary complications, choosing a therapy for an individual, predicting response to a therapy for an individual, determining the efficacy of a therapy in an individual, and/or determining the prognosis for an individual. That is that cells other than cells associated with a condition (e.g. cancer cells) are in fact reflective of the condition process. For instance, in the case of cancer, infiltrating immune cells can determine the outcome of the disease. In another aspect, a combination of information from a cancer cell plus responding immune cells in the blood of a cancer patient can be used for diagnosis or prognosis of the cancer.

### Conditions

The methods of the invention are applicable to any condition in an individual involving, indicated by, and/or arising from, in whole or in part, altered physiological status in a cell. The term “physiological status” includes mechanical, physical, and biochemical functions in a cell. In some embodiments, the physiological status of a cell is determined by measuring characteristics of cellular components of a cellular pathway. Cellular pathways are well known in the art. In some embodiments the cellular pathway is a signaling pathway. Signaling pathways are also well known in the art (see, e.g., Hunter T., Cell (2000) 100(1): 113-27; Pawson T, Kofler M Curr Opin Cell Biol. 2009 April; 21(2):147-53 Cell Signaling Technology, Inc., 2002 Catalogue, Pathway Diagrams pgs. 232-253). A condition involving or characterized by altered physiological status may be readily identified, for example, by determining the state in a cell of one or more activatable elements, as taught herein.

In some embodiments, the condition is CLL. In some embodiments, CLL is defined by a monoclonal B cell population that may co-express the following markers alone or in all possible combinations: CD5, CD 20, CD19, CD22, CD23, CD38, and CD45. Other arrangements include CDCD5 with CD19 and CD23 or CD5 with CD20 and CD23 and by surface immunoglobulin expression. In some embodiments, CLL is defined by a monoclonal B cell population that co-expresses CD5 with CD19 and CD23 or CD5 with CD20 and CD23 and dim surface immunoglobulin expression. In some embodiments, the level of expression of the B cell receptor is also measured including its components such as IgM, IgG, IgI, IgD, kappa chain, lambda chain, Igα (CD79α)/Igβ (CD79β).

CLL is a clonal B cell disorder with an incidence of about 15,000 cases/yr and is the most common leukemia in western countries. The disease is first suspected by presence of lymphocytosis greater than 4,000 μl wbcs. Its phenotypic characterization shows CD5+, CD19+, CD20+, and CD23+. Clonality is determined by mutually exclusive expression of lambda or kappa light chains. Disease staging systems introduces by Rai and Binet are based on clinically determinable features. Cytogenetic changes associated with poor clinical outcome include 11 q22-23 deletion, 17p deletion, trisomy 12, and p53 dysfunction which is through 17p deletion as one dominant mechanism. Cytogenetic changes associated with benign clinical course include the 13q14 deletion. Molecular markers include IgVH, CD 38, and ZAP-70.

One embodiment of the invention is directed to tumors and autoimmune diseases generally. Another embodiment of the invention relates to solid tumors and hematopoietic tumors. Other conditions within the scope of the present invention include, but are not limited to, cancers such as gliomas, lung cancer, colon cancer and prostate cancer. Specific signaling pathway alterations have been described for many cancers, including loss of PTEN and resulting activation of Akt signaling in prostate cancer (Whang Y E. Proc Natl Acad Sci USA Apr. 28, 1998; 95(9):5246-50), increased IGF-1 expression in prostate cancer (Schaefer et al., Science Oct. 9, 1998, 282: 199a), EGFR over expression and resulting ERK activation in glioma cancer (Thomas C Y. Int J Cancer Mar. 10, 2003; 104(1):19-27), expression of HER2 in breast cancers (Menard et al. Oncogene. Sep. 29 2003, 22(42):6570-8), and APC mutation and activated Wnt signaling in colon cancer (Bienz M. Curr Opin Genet Dev 1999 October, 9(5):595-603).

Diseases other than cancer involving altered physiological status are also encompassed by the present invention. For example, it has been shown that diabetes involves underlying signaling changes, namely resistance to insulin and failure to activate downstream signaling through IRS (Burks D J, White M F. Diabetes 2001 February; 50 Suppl 1:S140-5). Similarly, cardiovascular disease has been shown to involve hypertrophy of the cardiac cells involving multiple pathways such as the PKC family (Malhotra A. Mol Cell Biochem 2001 September; 225 (1-):97-107). Inflammatory diseases, such as rheumatoid arthritis, are known to involve the chemokine receptors and disrupted downstream signaling (D'Ambrosio D. J Immunol Methods 2003 February; 273 (1-2):3-13). The invention is not limited to diseases presently known to involve altered cellular function, but includes diseases subsequently shown to involve physiological alterations or anomalies.

In some embodiments, the present invention is directed to methods for classifying one or more cells in a sample derived from an individual having or suspected of having condition. In some embodiments, the invention allows for identification of prognostically and therapeutically relevant subgroups of the conditions and prediction of the clinical course of an individual. In some embodiments, the invention provides method of classifying a cell according to the activation level of one or more activatable element in a cell from an individual having or suspected of having condition. In some embodiments, the classification includes classifying the cell as a cell that is correlated with a clinical outcome. The clinical outcome can be the prognosis and/or diagnosis of a condition, and/or staging or grading of a condition. In some embodiments, the classifying of the cell includes classifying the cell as a cell that is correlated to a patient response to a treatment. In some embodiments, the classifying of the cell includes classifying the cell as a cell that is correlated with minimal residual disease or emerging resistance.

### Activatable Elements

The methods and compositions of the invention may be employed to examine and profile the status of any activatable element in a cellular pathway, or collections of such activatable elements. Single or multiple distinct pathways may be profiled (sequentially or simultaneously), or subsets of activatable elements within a single pathway or across multiple pathways may be examined (again, sequentially or simultaneously).

As will be appreciated by those in the art, a wide variety of activation events can find use in the present invention. In general, the basic requirement is that the activation results in a change in the activatable protein that is detectable by some indication (termed an “activation state indicator”), preferably by altered binding of a labeled binding element or by changes in detectable biological activities (e.g., the activated state has an enzymatic activity which can be measured and compared to a lack of activity in the non-activated state). What is important is to differentiate, using detectable events or moieties, between two or more activation states (e.g. “off” and “on”).

The activation state of an individual activatable element is either in the on or off state. As an illustrative example, and without intending to be limited to any theory, an individual phosphorylatable site on a protein can activate or deactivate the protein. The terms “on” and “off,” when applied to an activatable element that is a part of a cellular constituent, are used here to describe the state of the activatable element, and not the overall state of the cellular constituent of which it is a part. Typically, a cell possesses a plurality of a particular protein or other constituent with a particular activatable element and this plurality of proteins or constituents usually has some proteins or constituents whose individual activatable element is in the on state and other proteins or constituents whose individual activatable element is in the off state. Since the activation state of each activatable element is measured through the use of a binding element that recognizes a specific activation state, only those activatable elements in the specific activation state recognized by the binding element, representing some fraction of the total number of activatable elements, will be bound by the binding element to generate a measurable signal. The measurable signal corresponding to the summation of individual activatable elements of a particular type that are activated in a single cell is the “activation level” for that activatable element in that cell.

Activation levels for a particular activatable element may vary among individual cells so that when a plurality of cells is analyzed, the activation levels follow a distribution. The distribution may be a normal distribution, also known as a Gaussian distribution, or it may be of another type. Different populations of cells may have different distributions of activation levels that can then serve to distinguish between the populations. In some embodiments, the basis for classifying cells is that the distribution of activation levels for one or more specific activatable elements will differ among different phenotypes. A certain activation level, or more typically a range of activation levels for one or more activatable elements seen in a cell or a population of cells, is indicative that that cell or population of cells belongs to a distinctive phenotype. Other measurements, such as cellular levels (e.g., expression levels) of biomolecules that may not contain activatable elements, may also be used to classify cells in addition to activation levels of activatable elements; it will be appreciated that these levels also will follow a distribution, similar to activatable elements. Thus, the activation level or levels of one or more activatable elements, optionally in conjunction with levels of one or more levels of biomolecules that may not contain activatable elements, of cell or a population of cells may be used to classify a cell or a population of cells into a class. Once the activation level of intracellular activatable elements of individual single cells is known they can be placed into one or more classes, e.g., a class that corresponds to a phenotype. A class encompasses a class of cells wherein every cell has the same or substantially the same known activation level, or range of activation levels, of one or more intracellular activatable elements. For example, if the activation levels of five intracellular activatable elements are analyzed, predefined classes that encompass one or more of the intracellular activatable elements can be constructed based on the activation level, or ranges of the activation levels, of each of these five elements. It is understood that activation levels can exist as a distribution and that an activation level of a particular element used to classify a cell may be a particular point on the distribution but more typically may be a portion of the distribution.

In addition to activation levels of intracellular activatable elements, expression levels of intracellular or extracellular biomolecules, e.g., proteins can be used alone or in combination with activation states of activatable elements to classify cells. Further, additional cellular elements, e.g., biomolecules or molecular complexes such as RNA, DNA, carbohydrates, metabolites, and the like, may be used in conjunction with activatable states or expression levels in the classification of cells encompassed here.

In some embodiments, other characteristics that affect the status of a cellular constituent may also be used to classify a cell. Examples include the translocation of biomolecules or changes in their turnover rates and the formation and disassociation of complexes of biomolecule. Such complexes can include multi-protein complexes, multi-lipid complexes, homo- or hetero-dimers or oligomers, and combinations thereof. Other characteristics include proteolytic cleavage, e.g. from exposure of a cell to an extracellular protease or from the intracellular proteolytic cleavage of a biomolecule.

Additional elements may also be used to classify a cell, such as the expression level of extracellular or intracellular markers, nuclear antigens, enzymatic activity, protein expression and localization, cell cycle analysis, chromosomal analysis, cell volume, and morphological characteristics like granularity, size and size of nucleus or other distinguishing characteristics. For example, B cells can be further subdivided based on the expression of cell surface markers such as B-cell receptor comprised of a membrane-bound form of a ligand binding moiety such as IgM, IgH, IgD, heavy chains non-covalently linked with kappa and lambda light chains and a signal transduction moiety which is heterodimer called Ig-a/Ig-13 (CD79), bound together by disulfide bridges. Each member of the dimer spans the plasma membrane and has a cytoplasmic tail bearing an immunoreceptor tyrosine-based activation (ITAM) motif transduction moiety. Additional elements that are BCR regulators, such as the following can be used to classify the cell: CD45, CD5, CD19, CD20, CD22, CD23, CD27, CD37, CD40, CD52, CD38, CD96, major histocompatability antigen (MHC) Class 1 or MHC Class 2.

Alternatively, predefined classes of cells can be classified based upon shared characteristics that may include inclusion in one or more additional predefined class or the presence of extracellular and/or intracellular markers, a similar gene expression profile, mutational status, epigenetic silencing, nuclear antigens, enzymatic activity, protein expression and localization, cell cycle analysis, chromosomal analysis, cell volume, and morphological characteristics like granularity and size of nucleus or other distinguishing characteristics.

In some embodiments, the physiological status of one or more cells is determined by examining and profiling the activation level of one or more activatable elements in a cellular pathway. In some embodiments, a cell is classified according to the activation level of a plurality of activatable elements. In some embodiments, a hematopoietic cell is classified according to the activation levels of a plurality of activatable elements. In some embodiments, the activation level of one or more activatable elements of a hematopoietic cell is correlated with a condition. In some embodiments, the activation level of one or more activatable elements of a hematopoietic cell is correlated with a neoplastic, autoimmune or hematopoietic condition as described herein. Examples of hematopoietic cells include but are not limited to pluripotent hematopoietic stem cells, myeloid progenitors, B-lymphocyte lineage progenitor or derived cells, T-lymphocyte lineage progenitor or derived cells, NK cell lineage progenitor or derived cells, granulocyte lineage progenitor or derived cells, monocyte lineage progenitor or derived cells, megakaryocyte lineage progenitor or derived cells and erythroid lineage progenitor or derived cells. In some embodiments, the hematopoietic cell is a B-lymphocyte lineage progenitor or derived cell as described herein.

In some embodiments, the activation level of one or more activatable elements in single cells within the sample is determined. Cellular constituents that may include activatable elements include without limitation, proteins, carbohydrates, lipids, nucleic acids and metabolites. The activatable element may be a portion of the cellular constituent, for example, an amino acid residue in a protein that may undergo phosphorylation, or it may be the cellular constituent itself, for example, a protein that is activated by translocation from one part of the cell to another, change in conformation (due to, e.g., change in pH or ion concentration), by proteolytic cleavage, and the like. Upon activation, a change occurs to the activatable element, such as covalent modification of the activatable element (e.g., binding of a molecule or group to the activatable element, including but not limited to, phosphorylation, acetylation, methylation, ubiquitination) or a conformational change. Such changes generally contribute to changes in particular biological, biochemical, or physical properties of the cellular constituent that contains the activatable element. The state of the cellular constituent that contains the activatable element is determined to some degree, though not necessarily completely, by the state of activation of a particular activatable element of the cellular constituent. For example, a protein may have multiple activatable elements, and the particular activation states of these elements may overall determine the activation state of the protein; the state of a single activatable element is not necessarily determinative. Additional factors, such as the binding of other proteins, pH, ion concentration, interaction with other cellular constituents, and the like, can also affect the state of the cellular constituent.

In some embodiments, the activation levels of a plurality of intracellular activatable elements in single cells are determined. In some embodiments, at least about 2, 3, 4, 5, 6, 7, 8, 9, 10 or more than 10 intracellular activatable elements are determined.

Activation states of activatable elements may result from chemical additions or modifications of biomolecules and include biochemical processes such as glycosylation, phosphorylation, acetylation, methylation, biotinylation, glutamylation, glycylation, hydroxylation, isomerization, prenylation, myristoylation, lipoylation, phosphopantetheinylation, sulfation, ISGylation, nitrosylation, palmitoylation, SUMOylation, ubiquitination, neddylation, citrullination, amidation, and disulfide bond formation, disulfide bond reduction. Other possible chemical additions or modifications of biomolecules include the formation of protein carbonyls, direct modifications of protein side chains, such as o-tyrosine, chloro-, nitrotyrosine, and dityrosine, and protein adducts derived from reactions with carbohydrate and lipid derivatives. Other modifications may be non-covalent, such as binding of a ligand or binding of an allosteric modulator.

Examples of proteins that may include activatable elements include, but are not limited to kinases, phosphatases, lipid signaling molecules, adaptor/scaffold proteins, cytokines, cytokine regulators, ubiquitination enzymes, adhesion molecules, cytoskeletal/contractile proteins, heterotrimeric G proteins, small molecular weight GTPases, guanine nucleotide exchange factors, GTPase activating proteins, caspases, proteins involved in apoptosis (e.g. PARP), cell cycle regulators, molecular chaperones, metabolic enzymes, vesicular transport proteins, hydroxylases, isomerases, deacetylases, methylases, demethylases, tumor suppressor genes, proteases, ion channels, molecular transporters, transcription factors/DNA binding factors, regulators of transcription, and regulators of translation. Examples of activatable elements, activation states and methods of determining the activation level of activatable elements are described in US Publication Number 20060073474 entitled “Methods and compositions for detecting the activation state of multiple proteins in single cells” and U.S. Pat. No. 7,393,656 entitled “Methods and compositions for risk stratification” the content of which are incorporate here by reference.

In some embodiments, the activatable element that is a protein is selected from the group consisting Exemplary signaling proteins include, but are not limited to, kinases, HER receptors, PDGF receptors, Kit receptor, FGF receptors, Eph receptors, Trk receptors, IGF receptors, Insulin receptor, Met receptor, Ret, VEGF receptors, TIE1, TIE2, FAK, Jak1, Jak2, Jak3, Tyk2, Src, Lyn, Fyn, Lyn, Fgr, Yes, Csk, Abl, Btk, ZAP-70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Weel, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3α, GSK3β, Cdks, CLKs, PKR, PI3-Kinase class 1, class 2, class 3, mTor, SAPK/JNK1,2,3, p38s, PKR, DNA-PK, ATM, ATR, phosphatases, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, SHIPs, myotubularins, lipid signaling, phosphoinositide kinases, phospholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, cytokines, IL-2, IL-4, IL-8, IL-6, interferon γ, interferon α, cytokine regulators, suppressors of cytokine signaling (SOCs), ubiquitination enzymes, Cbl, SCF ubiquitination ligase complex, APC/C, adhesion molecules, integrins, Immunoglobulin-like adhesion molecules, selectins, cadherins, catenins, focal adhesion kinase, p130CAS, cytoskeletal/contractile proteins, fodrin, actin, paxillin, myosin, myosin binding proteins, tubulin, eg5/KSP, CENPs, heterotrimeric G proteins, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, guanine nucleotide exchange factors, Vav, Tiam, Sos, Dbl, PRK, TSC1,2, GTPase activating proteins, Ras-GAP, Arf-GAPs, Rho-GAPs, caspases, Caspase 2, Caspase 3, Caspase 6, Caspase 7, Caspase 8, Caspase 9, PARP, proteins involved in apoptosis, Bcl-2, Mcl-1, Bcl-XL, Bcl-w, Bcl-B, A1, Bax, Bak, Bok, Bik, Bad, Bid, Bim, Bmf, Hrk, Noxa, Puma, IAPB, XIAP, Smac, cell cycle regulators, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, vesicular transport proteins, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, isomerases, Pin1 prolyl isomerase, topoisomerases, deacetylases, Histone deacetylases, sirtuins, acetylases, histone acetylases, CBP/P300 family, MYST family, ATF2, methylases, DNA methyl transferases, demethylases, Histone H3K4 demethylases, H3K27, JHDM2A, UTX, tumor suppressor genes, VHL, WT-1, p53, Hdm, PTEN, proteases, ubiquitin proteases, urokinase-type plasminogen activator (uPA) and uPA receptor (uPAR) system, cathepsins, metalloproteinases, esterases, hydrolases, separase, ion channels, potassium channels, sodium channels, molecular transporters, multi-drug resistance proteins, P-Glycoprotein, nucleoside transporters, transcription factors/DNA binding proteins, Ets, Elk, SMADs, Rel-A (p65-NFKB), CREB, NFAT, ATF-2, AFT, Myc, Fos, Sp1, Egr-1, T-bet, HIFs, FOXOs, E2Fs, SRFs, TCFs, Egr-1, β-catenin, FOXO STAT1, STAT3, STAT4, STAT5, STAT6, p53, WT-1, HMGA, regulators of translation, pS6, 4EPB-1, eIF4E-binding protein, regulators of transcription, RNA polymerase, initiation factors, and elongation factors.

In some embodiments the protein is selected from the group consisting of PI3-Kinase (p85, p110a, p110b, p110d), Jak1, Jak2, SOCs, Rac, Rho, Cdc42, Ras-GAP, Vav, Tiam, Sos, Dbl, Nck, Gab, PRK, SHPT, and SHP2, SHIP1, SHIP2, sSHIP, PTEN, Shc, Grb2, PDK1, SGK, Akt1, Akt2, Akt3, TSC1,2, Rheb, mTor, 4EBP-1, p70S6Kinase, S6, LKB-1, AMPK, PFK, Acetyl-CoAa Carboxylase, DokS, Rafs, Mos, Tp12, MEK1/2, MLK3, TAK, DLK, MKK3/6, MEKK1,4, MLK3, ASK1, MKK4/7, SAPK/JNK1,2,3, p38s, Erk1/2, Syk, Btk, BLNK, LAT, ZAP-70, Lyn, Cbl, SLP-76, PLCγ₁, PLCγ2, STAT1, STAT2, STAT3, STAT4, STAT5a, STAT5b, STAT6, FAK, p130CAS, PAKs, LIMK1/2, Hsp90, Hsp70, Hsp27, SMADs, Rel-A (p65-NFKB), CREB, Histone H₂B, HATs, HDACs, PKR, Rb, Cyclin D, Cyclin E, Cyclin A, Cyclin B, P16, p14Arf, p27KIP, p21CIP, Cdk4, Cdk6, Cdk7, Cdk1, Cdk2, Cdk9, Cdc25, A/B/C, Abl, E2F, FADD, TRADD, TRAF2, RIP, Myd88, BAD, Bcl-2, Mcl-1, Bcl-XL, Caspase 2, Caspase 3, Caspase 6, Caspase 7, Caspase 8, Caspase 9, PARP, IAPB, Smac, Fodrin, Actin, Src, Lyn, Fyn, Lyn, NIK, IκB, p65(RelA), IKKα, PKA, PKCα, PKCβ, PKCθ, PKδ, CAMK, Elk, AFT, Myc, Egr-1, NFAT, ATF-2, Mdm2, p53, DNA-PK, Chk1, Chk2, ATM, ATR, \tilde{β}catenin, CrkL, GSK3α, GSK3β, and FOXO.

In some embodiments, the classification of a cell according to activation level of an activatable element, e.g., in a cellular pathway comprises classifying the cell as a cell that is correlated with a clinical outcome. In some embodiments, the clinical outcome is the prognosis and/or diagnosis of a condition. In some embodiments, the clinical outcome is the presence or absence of a neoplastic, autoimmune or a hematopoietic condition. In some embodiments, the clinical outcome is the staging or grading of a neoplastic, autoimmune or hematopoietic condition. Examples of staging include, but are not limited to, aggressive, indolent, benign, refractory, Roman Numeral staging, TNM Staging, Rai staging, Binet staging, WHO classification, FAB classification, IPSS score, WPSS score, limited stage, extensive stage, staging according to cellular markers such as ZAP-70, IgV_{H} mutational status and CD38, occult, including information that may inform on time to progression, progression free survival, overall survival, or event-free survival.

In some embodiments, methods and compositions are provided for the classification of a cell according to the activation level of an activatable element, e.g., in a cellular pathway wherein the classification comprises classifying a cell as a cell that is correlated to a patient response to a treatment. In some embodiments, the patient response is selected from the group consisting of complete response, partial response, nodular partial response, no response, progressive disease, stable disease and adverse reaction.

In some embodiments, methods and compositions are provided for the classification of a cell according to the activation level of an activatable element, e.g., in a cellular pathway wherein the classification comprises classifying the cell as a cell that is correlated with minimal residual disease or emerging resistance.

In some embodiments, methods and compositions are provided for the classification of a cell according to the activation level of an activatable element, e.g., in a cellular pathway wherein the classification comprises selecting a method of treatment. Example of methods of treatments include, but are not limited to, chemotherapy, biological therapy, radiation therapy, bone marrow transplantation, Peripheral stem cell transplantation, umbilical cord blood transplantation, autologous stem cell transplantation, allogeneic stem cell transplantation, syngeneic stem cell transplantation, surgery, induction therapy, maintenance therapy, and watchful waiting.

Generally, the methods of the invention involve determining the activation levels of an activatable element in a plurality of single cells in a sample.

A. Signaling Pathways

In some embodiments, the methods of the invention are employed to determine the status of an activatable element in a signaling pathway. In some embodiments, a cell is classified, as described herein, according to the activation level of one or more activatable elements in one or more signaling pathways. Signaling pathways and their members have been extensively described. See (Hunter T. Cell (2000) 100(1): 13-27). Exemplary signaling pathways include the following pathways and their members: The MAP kinase pathway including Ras, Raf, MEK, ERK and elk; the PI3K/Akt pathway including PI-3-kinase, PDK1, Akt and Bad; the canonical and non-canonical NF-κB pathway including Nik, IKKs, IkB and NF-κB and the Wnt pathway including frizzled receptors, beta-catenin, APC and other co-factors and TCF (see Cell Signaling Technology, Inc. 2002 Catalog pages 231-279 and Hunter T., supra.). In some embodiments of the invention, the correlated activatable elements being assayed (or the signaling proteins being examined) are members of the MAP kinase, Akt, NFkB, WNT, STAT and/or PKC signaling pathways. The methods of the invention also comprise the methods, signaling pathways and signaling molecules disclosed in U.S. 61/085,789 which is hereby incorporated by reference in its entirety.

In some embodiments, the methods of the invention are employed to determine the status of a signaling protein in a signaling pathway known in the art including those described herein. Exemplary types of signaling proteins within the scope of the present invention include, but are not limited to, kinases, kinase substrates (i.e. phosphorylated substrates), phosphatases, phosphatase substrates, binding proteins (such as 14-3-3), receptor ligands and receptors (cell surface receptor tyrosine kinases and nuclear receptors)). Kinases and protein binding domains, for example, have been well described (see, e.g., Cell Signaling Technology, Inc., 2002 Catalogue “The Human Protein Kinases” and “Protein Interaction Domains” pgs. 254-279). Signaling proteins are identified above as activatable elements.

MAP kinase pathway: In some embodiments, the methods of the invention are employed to determine the status of an activatable element in the MAP kinase pathway. Without intending to be limited to any theory, the MAP Kinase pathway is a signal transduction pathway that couples intracellular responses to the binding of growth factors to cell surface receptors. This pathway is very complex and includes many protein components. In many cell types, activation of this pathway promotes cell division.

PI3K/Akt pathway: In some embodiments, the methods of the invention are employed to determine the status of an activatable element in a PI3K/Akt pathway. Without intending to be limited to any theory, the PI3K/Akt pathway plays a role in effecting alterations in abroad range of cellular functions in response to extracellular signals. A downstream effector of PI3K is the serine-threonine kinase Akt which in response to PI3K activation phosphorylates and regulates the activity of a number of targets including kinases, transcription factors and other regulatory molecules. The serine/threonine kinase Akt functions intracellularly as a nodal point for a constellation of converging upstream signaling pathways, which involve stimulation of receptor tyrosine kinases such as IGF-1R, HER2/Neu, VEGF-R, PDGF-R), and an assembly of membrane-localized complexes of receptor-PI3K and activation of Akt through the second messenger PIP3. The integration of these intracellular signals at the level of Akt and its kinase activity, regulates the phosphorylation of its several downstream effectors, such as NF-B, mTOR, Forkhead, Bad, GSK-3 and MDM-2. These phosphorylation events, in turn, mediate the effects of Akt on cell growth, proliferation, protection from pro-apoptotic stimuli, and stimulation of neoangiogenesis. Akt and its upstream regulators are deregulated in a wide range of solid tumors and hematologic malignancies. The Akt pathway is the central cell survival pathway that is activated by such oncogenic events as over expression of an upstream receptor tyrosine kinase such as EGFR (ibid) or loss of an upstream regulatory protein such as PTEN (ibid).

NE-κB pathway: In some embodiments, the methods of the invention are employed to determine the status of an activatable element in a NF-κB pathway. Without intending to be limited to any theory, the NF-KB pathway is involved in regulating many aspects of cellular activity, in stress, injury and especially in pathways of the immune response. Some examples are the response to and induction of IL-2, the induction of TAP1 and MHC molecules by NF-κB, and many aspects of the inflammatory response, e.g. induction of IL-1 (alpha and beta), TNF-alpha and leukocyte adhesion molecules (E-selectin, VCAM-1 and ICAM-1). Moreover, NF-κB is involved in many aspects of cell growth, differentiation and proliferation via the induction of certain growth and transcription factors (e.g. c-myc, ras and p53). The NF-κB signal transduction pathway is misregulated in a variety of human cancers, especially those of lymphoid cell origin. Several human lymphoid cancer cells are reported to have mutations or amplifications of genes encoding NF-κB transcription factors. In most cancer cells NF-κB is constitutively active and resides in the nucleus. In some cases, this may be due to chronic stimulation of the IKK pathway, while in others the gene encoding IkBa may be defective. Such continuous nuclear NF-κB activity not only protects cancer cells from apoptotic cell death, but may even enhance their growth activity. Designing anti-tumor agents to block NF-κB activity or to increase their sensitivity to conventional chemotherapy may have great therapeutic value.

WNT pathway: In some embodiments, the methods of the invention are employed to determine the status of an activatable element in a WNT pathway. Without intending to be limited to any theory, the Wnt signaling pathway describes a complex network of proteins most well known for their roles in embryogenesis and cancer, but also involved in normal physiological processes in adult animals. The canonical Wnt pathway describes a series of events that occur when Wnt proteins bind to cell-surface receptors of the Frizzled family, causing the receptors to activate Dishevelled family proteins and ultimately resulting in a change in the amount of β-catenin that reaches the nucleus. Dishevelled (DSH) is a key component of a membrane-associated Wnt receptor complex which, when activated by Wnt binding, inhibits a second complex of proteins that includes axin, GSK-3, and the protein APC. The axin/GSK-3/APC complex normally promotes the proteolytic degradation of the β-catenin intracellular signaling molecule. After this “β-catenin destruction complex” is inhibited, a pool of cytoplasmic β-catenin stabilizes, and some β-catenin is able to enter the nucleus and interact with TCF/LEF family transcription factors to promote specific gene expression.

PKC pathway: In some embodiments, the methods of the invention are employed to determine the status of an activatable element in a PKC pathway. Without intending to be limited to any theory, PKC pathway is associated with cell proliferation, differentiation, and apoptosis. At least eleven closely related PKC isozymes have been reported that differ in their structure, biochemical properties, tissue distribution, subcellular localization, and substrate specificity. They are classified as conventional (α, β1, β2, γ), novel (δ, ε, η, θ, μ), and atypical (ζ, λ) isozymes. Conventional PKC isozymes are Ca2+-dependent, while novel and atypical isozymes do not require Ca2+ for their activation. All PKC isozymes, with the exception of ζ and λ, are activated by diacylglycerol (DAG). PKC isozymes negatively or positively regulate critical cell cycle transitions, including cell cycle entry and exit and the G1 and G2 checkpoints. Altered PKC activity has been linked with various types of malignancies. Higher levels of PKC and differential activation of various PKC isozymes have been reported in breast tumors, adenomatous pituitaries, thyroid cancer tissue, leukemic cells, and lung cancer cells. Down regulation of PKCα is reported in the majority of colon adenocarcinomas and in the early stages of intestinal carcinogenesis. Thus, PKC inhibitors have become important tools in the treatment of cancers. The involvement of PKC in the regulation of apoptosis adds another dimension to the effort to develop drugs that will specifically target PKC. PKC pathway activation is thought to also play a role in diseases such as cardiovascular disease and diabetes.

In some embodiments of the invention, the methods described herein are employed to determine the status of an activatable element in a signaling pathway. Methods and compositions are provided for the classification of a cell according to the status of an activatable element in a signaling pathway. The cell can be a hematopoietic cell. Examples of hematopoietic cells are described above.

In some embodiments, the classification of a cell according to the status of an activatable element in a signaling pathway comprises classifying the cell as a cell that is correlated with a clinical outcome. In some embodiments, the clinical outcome is the prognosis and/or diagnosis of a condition. In some embodiments, the clinical outcome is the presence or absence of a neoplastic, autoimmune or a hematopoietic condition. In some embodiments, the clinical outcome is the staging or grading of a neoplastic, autoimmune or hematopoietic condition. Examples of staging include, but are not limited to, aggressive, indolent, benign, refractory, Roman Numeral staging, TNM Staging, Rai staging, Binet staging, WHO classification, FAB classification, IPSS score, WPSS score, limited stage, extensive stage, staging according to cellular markers such as ZAP-70, IgV_{H} mutational status and CD38, occult, including information that may inform on time to progression, progression free survival, overall survival, or event-free survival.

In some embodiments, methods and compositions are provided for the classification of a cell according to the status of an activatable element in a signaling pathway wherein the classification comprises classifying a cell as a cell that is correlated to a patient response to a treatment. In some embodiments, the patient response is selected from the group consisting of complete response, partial response, nodular partial response, no response, progressive disease, stable disease and adverse reaction.

In some embodiments, methods and compositions are provided for the classification of a cell according to the status of an activatable element in a signaling pathway wherein the classification comprises classifying the cell as a cell that is correlated with minimal residual disease or emerging resistance.

The invention is not limited to presently elucidated signaling pathways and signal transduction proteins, and encompasses signaling pathways and proteins subsequently identified.

B. B-Cell Receptor Pathway

In some embodiments, the methods and compositions of the invention may be employed to examine and profile the status of any activatable element in B-Cell Receptor (BCR) signaling, or collections of such activatable elements in a B-lymphocyte lineage progenitor or derived cell. In some embodiments, the physiological status of one or more B-lymphocyte lineage progenitor or derived cell is determined by examining and profiling the status of one or more activatable element in BCR signaling. In some embodiments, a B-lymphocyte lineage progenitor or derived cell is classified, as described herein, according to the activation level of one or more activatable elements in BCR signaling. Examples of B-lymphocyte lineage derived cell include, but are not limited to, B-lymphocyte lineage early pro-B cell, late pro-B cell, large pre-B cell, small pre-B cell, immature B cell, mature B cell, plasma cell, memory B cell, a CD5+ B cell, a CD38+ B cell, a B cell bearing a mutated or non mutated heavy chain of the B cell receptor and a B cell expressing ZAP-70. In some embodiments, the B-lymphocyte lineage progenitor or derived cell is a cell associated with a condition as described herein.

Without intending to be limited to any theory, BCR cross-linking triggers phosphorylation of tyrosines within the ITAM motif domains of Iga and 103 by Src family member tyrosine kinases (e.g., Lyn, Lyn, Blk, Fyn). The phosphorylated ITAMs of Igαβ recruit and enhance phosphorylation of Syk (directly) and Btk (via Syk). BCR cross-linking also brings together numerous regulator and adapter molecules (e.g., SLP-65/BLNK, Grb2, CD22, SHP-1) and compartmentalizes the BCR in lipid rafts with coreceptors CD19 and CD21. Following Syk and Btk activation, the enzymes phospholipase-Cγ2 (PLCγ2) and PI3K propagate BCR signaling. PLCγ2 activation generates calcium flux, inositol-1,4,5-triphosphate, and diacylglycerol, and results in activation of protein kinase C and NF-κB. Syk interacts with PLCγ2 via adapters, whereas Btk can interact directly, and each is required for PLCγ2 activity following BCR cross-linking Both Syk and Btk can activate PI3K following BCR cross-linking Activation of PI3K enables Akt-mediated survival signaling, and PI3K is required for BCR-mediated survival during B cell development. PLCγ2 and PI3K also initiate kinase cascades that result in phosphorylation of the MAPK family proteins ERK1/2 and p38. Activation of the Ras-Raf-ERK1/2 signaling cascade is considered a central event in BCR signaling, and decreased Ras activation due to RasGRP1 and RasGRP3 loss in mouse impairs B cell proliferation. In contrast, p38 is a stress response protein that interacts with p53 and regulates cell cycle checkpoints. Differential activation of ERK1/2 and p38 might enable the BCR to drive diverse cellular outcomes, but the question arises whether a given B cell activates these two pathways simultaneously or favors one pathway depending on additional signaling context.

Efficient activation of BCR signaling depends on generation of H₂O₂ and inactivation of negative regulatory protein tyrosine phosphatases (PTPs). Following BCR cross-linking, recruitment and activation of calcium-dependent NADPH oxidases (NOX) proteins, such as NOX5, enables production of H₂O₂ and lowers the signaling threshold for the BCR. BCR-induced H₂O₂ transiently inactivates membrane proximal PTPs, including SHP-1, via reversible oxidation of the catalytic cysteine to sulfenic acid. Elegant work reconstituting the BCR signaling pathway in insect cells has suggested a model of redox feedback loops where H₂O₂ inactivates PTPs and enables amplification of early signaling events, such as Syk phosphorylation and ITAM binding. Recent work characterized endogenously generated H₂O₂ as the primary redox species generated by BCR signaling and indicated that NOX-dependent production of H₂O₂ was critical to initiate a wave of BCR signaling in mouse A20 B cells.

In some embodiments, the invention provides a method for classifying a B-lymphocyte lineage progenitor or derived cell upon treatment with a modulator and/or inhibitor. Examples of B-lymphocyte lineage progenitor or derived cells include, but are not limited to an early pro-B cell, late pro-B cell, large pre-B cell, small pre-B cell, immature B cell, mature B cell, plasma cell and memory B cell, a CD5+ B cell, a CD38+ B cell, a B cell bearing a mutated or non mutated heavy chain of the B cell receptor, or a B cell expressing ZAP-70.

In some embodiments, the classification includes classifying the cell according to the status of an activatable element in a BCR pathway as a cell that is correlated with a clinical outcome. In some embodiments, the invention provides methods for classifying a B-lymphocyte lineage progenitor or derived cell based on an alteration in signaling proximal to the BCR. In some embodiments, the clinical outcome is the prognosis and/or diagnosis of a condition. In some embodiments, the clinical outcome is the presence or absence of a neoplastic, autoimmune or a hematopoietic condition, such as Chronic Lymphocytic Leukemia (CLL), B lymphocyte lineage leukemia, B lymphocyte lineage lymphoma, Multiple Myeloma, or plasma cell disorders, e.g., amyloidosis or Waldenstrom's macroglobulinemia. In some embodiments, the condition is CLL. In some embodiments, the invention provides methods for classifying a CLL cell based on an alteration in signaling proximal to the BCR. The presence of the alteration is indicative of a clinical outcome. In some embodiments, CLL is defined by a monoclonal B cell population that may co-express the following markers alone or in all possible combinations: CD5, CD 20, CD19, CD22, CD23, CD38, and CD45. Other arrangements include CDCD5 with CD19 and CD23 or CD5 with CD20 and CD23 and by surface immunoglobulin expression. In some embodiments, CLL is defined by a monoclonal B cell population that co-expresses CD5 with CD19 and CD23 or CD5 with CD20 and CD23 and dim surface immunoglobulin expression. Additional B-cell markers can be used to identify or classify a B-lymphocyte lineage progenitor or derived cell. Non-limiting examples such as the following can be used to classify the cell: CD45, CD5, CD19, CD20, CD22, CD23, CD27, CD37, CD40, CD52, CD38, CD96, major histocompatability antigen (MHC) Class 1 or MHC Class 2.

In some embodiments of the methods of the invention, the classifying of the B-lymphocyte lineage progenitor or derived cell based on activation level of an activatable element in BCR pathway includes classifying the cell as a cell that is correlated to a patient response to a treatment, as defined above.

In some embodiments of the methods of the invention, the classifying of the B-lymphocyte lineage progenitor or derived cells based on activation of an activatable element in BCR pathway includes classifying the cell as a cell that is correlated with minimal residual disease or emerging resistance.

C. Tonic Signaling

In some embodiments, the methods and compositions of the invention may be employed to determine the status of a tonic signaling pathway in a cell. In some embodiments, the methods and compositions of the invention may be employed to examine and profile the status of any activatable element in a tonic signaling pathway, or collections of such activatable elements in a cell. In some embodiments, the physiological status of a cell is determined by examining and profiling the status of one or more activatable elements in a tonic signaling pathway. In some embodiments, a cell is classified, as described herein, according to the status of one or more activatable elements in a tonic signaling pathway. The term “tonic signaling” includes ligand-independent signaling, antigen independent signaling, basal signaling, signaling in the resting stae, and non-induced or ligand-independent signaling.

Without intending to be limited to any theory, recent evidence supports the notion that in most signal transduction systems regulated by cellular receptors some basal level of signaling occurs continuously in a ligand-independent manner, although the flux through such systems may vary considerably. The basal, tonic, or the steady state level of signaling in unstimulated cells is the result of equilibrium of positive and negative regulators within a signaling pathway. Thus, the balanced actions of positive and negative regulators of signal transduction set the steady state equilibrium. The steady state level of signaling in the unstimulated state may itself have functional consequences, for instance, to maintain certain differentiated cellular properties or functions.

In some embodiments, the invention provides for methods of determining tonic signaling status of a cell. Methods and compositions are provided for the classification of a cell according to the status of an activatable element in a tonic signaling pathway. The cell can be a hematopoietic cell. Examples of hematopoietic cells are described above.

In some embodiments, the classification of a cell according to the status of an activatable element in a tonic signaling pathway comprises classifying the cell as a cell that is correlated with a clinical outcome. In some embodiments, the clinical outcome is the prognosis and/or diagnosis of a condition. In some embodiments, the clinical outcome is the presence or absence of a neoplastic, autoimmune or a hematopoietic condition. Examples of neoplastic, autoimmune or hematopoietic conditions include, but are not limited to, such as Chronic Lymphocytic Leukemia (CLL), B lymphocyte lineage leukemia, B lymphocyte lineage lymphoma, Multiple Myeloma, or plasma cell disorders, e.g., amyloidosis or Waldenstrom's macroglobulinemia. In some embodiments, the condition is CLL. In some embodiments, CLL is defined by a monoclonal B cell population that co-expresses CD5 with CD19 and CD23 or CD5 with CD20 and CD23 and by surface immunoglobulin expression.

In some embodiments, the clinical outcome is the staging or grading of a neoplastic, autoimmune or hematopoietic condition. Examples of staging are defined above.

In some embodiments, the invention provides methods for classifying a CLL cell based on an alteration in signaling proximal to the BCR that is indicative of the presence of tonic signaling. The presence of the alteration is indicative of a clinical outcome, where the clinical outcome is as described herein.

In some embodiments, methods and compositions are provided for the classification of a cell according to the status of an activatable element in a tonic signaling pathway wherein the classification comprises classifying a cell as a cell that is correlated to a patient response to a treatment. In some embodiments, the patient response is selected from the group consisting of complete response, partial response, nodular partial response, no response, progressive disease, stable disease and adverse reaction.

In some embodiments, methods and compositions are provided for the classification of a cell according to the status of an activatable element in a tonic signaling pathway wherein the classification comprises classifying the cell as a cell that is correlated with minimal residual disease or emerging resistance.

In some embodiments, methods and compositions are provided for the classification of a cell according to the status of an activatable element in a tonic signaling pathway wherein the classification comprises selecting a method of treatment. Examples of methods of treatments are described above.

### Binding Element

In some embodiments of the invention, the activation level of an activatable element is determined by contacting a cell with a binding element that is specific for an activation state of the activatable element. The term “Binding element” includes any molecule, e.g., peptide, polypeptide (including an antibody) nucleic acid, small organic molecule which is capable of detecting an activation state of an activatable element over another activation state of the activatable element. See U.S. Ser. No. 12/229,976 which is incorporated by reference in its entirety.

In some embodiments, the binding element is a peptide, polypeptide, oligopeptide or a protein. The peptide, polypeptide, oligopeptide or protein may be made up of naturally occurring amino acids and peptide bonds, or synthetic peptidomimetic structures. Thus “amino acid”, or “peptide residue”, as used herein include both naturally occurring and synthetic amino acids. For example, homo-phenylalanine, citrulline and norleucine are considered amino acids for the purposes of the invention. The side chains may be in either the (R) or the (S) configuration. In some embodiments, the amino acids are in the (S) or L-configuration. If non-naturally occurring side chains are used, non-amino acid substituents may be used, for example to prevent or retard in vivo degradation. Proteins including non-naturally occurring amino acids may be synthesized or in some cases, made recombinantly; see van Hest et al., FEBS Lett 428:(1-2) 68-70 May 22, 1998 and Tang et al., Abstr. Pap Am. Chem. 5218: U138 Part 2 Aug. 22, 1999, both of which are expressly incorporated by reference herein.

Methods of the present invention may be used to detect any particular activatable element in a sample that is antigenically detectable and antigenically distinguishable from other activatable element which is present in the sample. For example, as demonstrated (see, e.g., the Examples) and described herein, the activation state-specific antibodies of the present invention can be used in the present methods to identify distinct signaling cascades of a subset or subpopulation of complex cell populations; and the ordering of protein activation (e g, kinase activation) in potential signaling hierarchies. Hence, in some embodiments the expression and phosphorylation of one or more polypeptides are detected and quantified using methods of the present invention. In some embodiments, the expression and phosphorylation of one or more polypeptides that are cellular components of a cellular pathway are detected and quantified using methods of the present invention. As used herein, the term “activation state-specific antibody” or “activation state antibody” or grammatical equivalents thereof, refer to an antibody that specifically binds to a corresponding and specific antigen. Preferably, the corresponding and specific antigen is a specific form of an activatable element. Also preferably, the binding of the activation state-specific antibody is indicative of a specific activation state of a specific activatable element.

In some embodiments, the binding element is an antibody. In some embodiment, the binding element is an activation state-specific antibody. In some embodiment, the binding element is a phospho-specific antibody.

As pointed out above, activation state specific antibodies can be used to detect kinase activity, however additional means for determining kinase activation are provided by the present invention. For example, substrates that are specifically recognized by protein kinases and phosphorylated thereby are known. Antibodies that specifically bind to such phosphorylated substrates but do not bind to such non-phosphorylated substrates (phospho-substrate antibodies) may be used to determine the presence of activated kinase in a sample.

Many antibodies, many of which are commercially available (for example, see Cell Signaling Technology, www.cellsignal.com, Millipore, eBioscience, Caltag, Santa Cruz Biotech, Abcam, BD Biosciences, Sigma and Anaspec) the contents which are incorporated herein by reference) have been produced which specifically bind to the phosphorylated isoform of a protein but do not specifically bind to a non-phosphorylated isoform of a protein. Many such antibodies have been produced for the study of signal transducing proteins which are reversibly phosphorylated. Particularly, many such antibodies have been produced which specifically bind to phosphorylated, activated isoforms of protein. Examples of proteins that can be analyzed with the methods described herein include, but are not limited to, kinases, HER receptors, PDGF receptors, Kit receptor, FGF receptors, Eph receptors, Trk receptors, IGF receptors, Insulin receptor, Met receptor, Ret, VEGF receptors, TIE1, TIE2, FAK, Jak1, Jak2, Jak3, Tyk2, Src, Lyn, Fyn, Lyn, Fgr, Yes, Csk, Abl, Btk, ZAP-70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Weel, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3a, GSK313, Cdks, CLKs, PKR, PI3-Kinase class 1, class 2, class 3, mTor, SAPK/JNK1,2,3, p38s, PKR, DNA-PK, ATM, ATR, phosphatases, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, SHIPs, myotubularins, lipid signaling, phosphoinositide kinases, phospholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, cytokines, IL-2, IL-4, IL-8, IL-6, interferon γ, interferon α, cytokine regulators, suppressors of cytokine signaling (SOCs), ubiquitination enzymes, Cbl, SCF ubiquitination ligase complex, APC/C, adhesion molecules, integrins, Immunoglobulin-like adhesion molecules, selectins, cadherins, catenins, focal adhesion kinase, p130CAS, cytoskeletal/contractile proteins, fodrin, actin, paxillin, myosin, myosin binding proteins, tubulin, eg5/KSP, CENPs, heterotrimeric G proteins, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, guanine nucleotide exchange factors, Vav, Tiam, Sos, Dbl, PRK, TSC1,2, GTPase activating proteins, Ras-GAP, Arf-GAPs, Rho-GAPs, caspases, Caspase 2, Caspase 3, Caspase 6, Caspase 7, Caspase 8, Caspase 9, PARP, proteins involved in apoptosis, Bcl-2, Mcl-1, Bcl-XL, Bcl-w, Bcl-B, A1, Bax, Bak, Bok, Bik, Bad, Bid, Bim, Bmf, Hrk, Noxa, Puma, IAPB, XIAP, Smac, cell cycle regulators, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, vesicular transport proteins, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, isomerases, Pin1 prolyl isomerase, topoisomerases, deacetylases, Histone deacetylases, sirtuins, acetylases, histone acetylases, CBP/P300 family, MYST family, ATF2, methylases, DNA methyl transferases, demethylases, Histone H3K4 demethylases, H3K27, JHDM2A, UTX, tumor suppressor genes, VHL, WT-1, p53, Hdm, PTEN, proteases, ubiquitin proteases, urokinase-type plasminogen activator (uPA) and uPA receptor (uPAR) system, cathepsins, metalloproteinases, esterases, hydrolases, separase, ion channels, potassium channels, sodium channels, molecular transporters, multi-drug resistance proteins, P-Glycoprotein, nucleoside transporters, transcription factors/DNA binding proteins, Ets, Elk, SMADs, Rel-A (p65-NFKB), CREB, NFAT, ATF-2, AFT, Myc, Fos, Sp1, Egr-1, T-bet, β-catenin, HIFs, FOXOs, E2Fs, SRFs, TCFs, Egr-1,13-catenin, FOXO STAT1, STAT3, STAT4, STAT5, STATE, p53, WT-1, HMGA, regulators of translation, pS6, 4EPB-1, eIF4E-binding protein, regulators of transcription, RNA polymerase, initiation factors, elongation factors. In some embodiments, the protein is S6.

Non-activation state antibodies may also be used in the present invention. In some embodiments, non-activation state antibodies bind to epitopes in both activated and non-activated forms of an element. Such antibodies may be used to determine the amount of non-activated plus activated element in a sample. In some embodiments, non-activation state antibodies bind to epitopes present in non-activated forms of an element but absent in activated forms of an element. Such antibodies may be used to determine the amount of non-activated element in a sample. Both types of non-activation state antibodies may be used to determine if a change in the amount of activation state element, for example from samples before and after treatment with a candidate bioactive agent as described herein, coincide with changes in the amount of non-activation state element. For example, such antibodies can be used to determine whether an increase in activated element is due to activation of non-activation state element, or due to increased expression of the element, or both.

A. Labels

The methods and compositions of the instant invention provide binding elements comprising a label or tag. By label is meant a molecule that can be directly (i.e., a primary label) or indirectly (i.e., a secondary label) detected; for example a label can be visualized and/or measured or otherwise identified so that its presence or absence can be known. A compound can be directly or indirectly conjugated to a label which provides a detectable signal, e.g. radioisotopes, fluorescers, enzymes, antibodies, particles such as magnetic particles, chemiluminescers, or specific binding molecules, etc. Specific binding molecules include pairs, such as biotin and streptavidin, digoxin and antidigoxin etc. Examples of labels include, but are not limited to, optical fluorescent and chromogenic dyes including labels, label enzymes and radioisotopes. See U.S. Ser. No. 12/229,976 which is incorporated by reference in its entirety.

In some embodiments, one or more binding elements are uniquely label. Using the example of two activation state specific antibodies, by “uniquely labeled” is meant that a first activation state antibody recognizing a first activated element comprises a first label, and second activation state antibody recognizing a second activated element comprises a second label, wherein the first and second labels are detectable and distinguishable, making the first antibody and the second antibody uniquely labeled.

In general, labels fall into four classes: a) isotopic labels, which may be radioactive or heavy isotopes; b) magnetic, electrical, thermal labels; c) colored, optical labels including luminescent, phosphorous and fluorescent dyes or moieties; and d) binding partners. Labels can also include enzymes (horseradish peroxidase, etc.) and magnetic particles. In some embodiments, the detection label is a primary label. A primary label is one that can be directly detected, such as a fluorophore.

Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, Texas Red, IAEDANS, EDANS, BODIPY FL, LC Red 640, Cy 5, Cy 5.5, LC Red 705 and Oregon green. Suitable optical dyes are described in the 1996 Molecular Probes Handbook by Richard P. Haugland, hereby expressly incorporated by reference. Suitable fluorescent labels also include, but are not limited to, green fluorescent protein (GFP; Chalfie, et al., Science 263(5148):802-805 (Feb. 11, 1994); and EGFP; Clontech—Genbank Accession Number U55762), blue fluorescent protein (BFP; 1. Quantum Biotechnologies, Inc. 1801 de Maisonneuve Blvd. West, 8th Floor, Montreal (Quebec) Canada H3H1J9; 2. Stauber, R. H. Biotechniques 24(3):462-471 (1998); 3. Heim, R. and Tsien, R. Y. Curr. Biol. 6:178-182 (1996)), enhanced yellow fluorescent protein (EYFP; 1. Clontech Laboratories, Inc., 1020 East Meadow Circle, Palo Alto, Calif. 94303), luciferase (Ichiki, et al., J. Immunol. 150(12):5408-5417 (1993)), .beta.-galactosidase (Nolan, et al., Proc Natl Acad Sci USA 85(8):2603-2607 (April 1988)) and Renilla WO 92/15673; WO 95/07463; WO 98/14605; WO 98/26277; WO 99/49019; U.S. Pat. No. 5,292,658; U.S. Pat. No. 5,418,155; U.S. Pat. No. 5,683,888; U.S. Pat. No. 5,741,668; U.S. Pat. No. 5,777,079; U.S. Pat. No. 5,804,387; U.S. Pat. No. 5,874,304; U.S. Pat. No. 5,876,995; and U.S. Pat. No. 5,925,558). All of the above-cited references are expressly incorporated herein by reference.

In some embodiments, labels for use in the present invention include: Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes) (Eugene, Oreg.), FITC, Rhodamine, and Texas Red (Pierce, Rockford, Ill.), Cy5, Cy5.5, Cy7 (Amersham Life Science, Pittsburgh, Pa.). Tandem conjugate protocols for Cy5PE, Cy5.5PE, Cy7PE, Cy5.5APC, Cy7APC are known in the art. Quantitation of fluorescent probe conjugation may be assessed to determine degree of labeling and protocols including dye spectral properties are also well known in the art. In some embodiments the fluorescent label is conjugated to an aminodextran linker which is conjugated to a binding element or antibody. Additional labels listed in and are available through the on-line and hard copy catalogues of BD Biosciences, Beckman Coulter, AnaSpec, Invitrogen, Cell Signaling Technology, Millipore, eBioscience, Caltag, Santa Cruz Biotech, Abcam and Sigma, the contents of which are incorporated herein by reference.

In some embodiments, the activatable elements are labeled with tags suitable for Inductively Coupled Plasma Mass Spectrometer (ICP-MS) as disclosed in Tanner et al. Spectrochimica Acta Part B: Atomic Spectroscopy, 2007 March; 62(3):188-195; Ornatsky et al, mRNA Detection in Leukemia Cell lines by Novel Metal-Tagged in situ Hybridization using Inductively Coupled Plasma Mass Spectometry, Translational Oncogenomics (2006):1, 1-9; Ornatsky et al, Multiple Cellular Antigen Detection by ICP-MS, J. Imm. Methods 308 (2006) 68-76; and Lou et al., Polymer-Based Elemental Tags for Sensitive Bioassays, Angew. Chem. Int. Ed., (2007) 46, 6111-6114.

Production of antibody-embedded substrates is well known; see Slinkin et al., Bioconj. Chem., 2:342-348 (1991); Torchilin et al., supra; Trubetskoy et al., Bioconj. Chem. 3:323-327 (1992); King et al., Cancer Res. 54:6176-6185 (1994); and Wilbur et al., Bioconjugate Chem. 5:220-235 (1994) (all of which are hereby expressly incorporated by reference), and attachment of or production of proteins with antigens is described above. Calmodulin-embedded substrates are commercially available, and production of proteins with CBP is described in Simcox et al., Strategies 8:40-43 (1995), which is hereby incorporated by reference in its entirety.

As will be appreciated by those in the art, tag-components of the invention can be made in various ways, depending largely upon the form of the tag. Components of the invention and tags are preferably attached by a covalent bond.

### Alternative Activation State Indicators

An alternative activation state indicator useful with the instant invention is one that allows for the detection of activation by indicating the result of such activation. For example, phosphorylation of a substrate can be used to detect the activation of the kinase responsible for phosphorylating that substrate. Similarly, cleavage of a substrate can be used as an indicator of the activation of a protease responsible for such cleavage. Methods are well known in the art that allow coupling of such indications to detectable signals, such as the labels and tags described above in connection with binding elements. For example, cleavage of a substrate can result in the removal of a quenching moiety and thus allowing for a detectable signal being produced from a previously quenched label.

### Modulators

In some embodiments, the methods and composition utilize a modulator. A modulator can be an activator, an inhibitor or a compound capable of impacting a cellular pathway. Modulators can take the form of environmental cues and inputs.

Modulation can be performed in a variety of environments. In some embodiments, cells are exposed to a modulator immediately after collection. In some embodiments where there is a mixed population of cells, purification of cells is performed after modulation. In some embodiments, whole blood is collected to which a modulator is added. In some embodiments, cells are modulated after processing for single cells or purified fractions of single cells. As an illustrative example, whole blood can be collected and processed for an enriched fraction of lymphocytes that is then exposed to a modulator. Modulation can include exposing cells to more than one modulator. For instance, in some embodiments, cells are exposed to at least 2, 3, 4, 5, 6, 7, 8, 9, or 10 modulators.

In some embodiments, cells are cultured post collection in a suitable media before exposure to a modulator. In some embodiments, the media is a growth media. In some embodiments, the growth media is a complex media that may include serum. In some embodiments, the growth media comprises serum. In some embodiments, the serum is selected from the group consisting of fetal bovine serum, bovine serum, human serum, porcine serum, horse serum, and goat serum. In some embodiments, the serum level ranges from 0.0001% to 30%. In some embodiments any suitable amount of serum is used. In some embodiments, the growth media is a chemically defined minimal media and is without serum. In some embodiments, cells are cultured in a differentiating media.

Modulators include chemical and biological entities, and physical or environmental stimuli. Modulators can act extracellularly or intracellularly. Chemical and biological modulators include growth factors, cytokines, neurotransmitters, adhesion molecules, hormones, small molecules, inorganic compounds, polynucleotides, antibodies, natural compounds, lectins, lactones, chemotherapeutic agents, biological response modifiers, carbohydrate, proteases and free radicals. Modulators include complex and undefined biologic compositions that may comprise cellular or botanical extracts, cellular or glandular secretions, physiologic fluids such as serum, amniotic fluid, or venom. Physical and environmental stimuli include electromagnetic, ultraviolet, infrared or particulate radiation, redox potential and pH, the presence or absences of nutrients, changes in temperature, changes in oxygen partial pressure, changes in ion concentrations and the application of oxidative stress. Modulators can be endogenous or exogenous and may produce different effects depending on the concentration and duration of exposure to the single cells or whether they are used in combination or sequentially with other modulators. Modulators can act directly on the activatable elements or indirectly through the interaction with one or more intermediary biomolecule. Indirect modulation includes alterations of gene expression wherein the expressed gene product is the activatable element or is a modulator of the activatable element.

In some embodiments, modulators produce different activation states depending on the concentration of the modulator, duration of exposure or whether they are used in combination or sequentially with other modulators.

In some embodiments the modulator is selected from the group consisting of growth factor, cytokine, adhesion molecule modulator, drugs, hormone, small molecule, polynucleotide, antibodies, natural compounds, lactones, chemotherapeutic agents, immune modulator, carbohydrate, proteases, ions, reactive oxygen species, peptides, and protein fragments, either alone or in the context of cells, cells themselves, viruses, and biological and non-biological complexes (e.g. beads, plates, viral envelopes, antigen presentation molecules such as major histocompatibility complex). In some embodiments, the modulator is a physical stimuli such as heat, cold, UV radiation, and radiation. Examples of modulators, include but are not limited to, F(ab)2 IgM, Rituxan, Alemtuzumab (such as Campath), anti CD22 (epratuzumab), anti-CD23 (lumiliximab), bendamustine, velcade, H₂O₂, phenylarsine oxide, sodium vanadate, H₂O₂, PMA, BAFF, April, SDF1a, CD40L, IGF-1, Imiquimod, polyCpG, fludarabine, cyclophosphamide, chlorambucil, IL-7, IL-6, IL-10, IL-27, IL-4, IL-2, IL-3, and thapsigargin.

In some embodiments, the modulator is an activator. In some embodiments the modulator is an inhibitor. In some embodiments, cells are exposed to one or more modulator. In some embodiments, cells are exposed to at least 2, 3, 4, 5, 6, 7, 8, 9, or 10 modulators. In some embodiments, cells are exposed to at least two modulators, wherein one modulator is an activator and one modulator is an inhibitor. In some embodiments, cells are exposed to at least 2, 3, 4, 5, 6, 7, 8, 9, or 10 modulators, where at least one of the modulators is an inhibitor.

In some embodiments, the modulator is a B cell receptor modulator. In some embodiments, the B cell receptor modulator is a B cell receptor activator. An example of B cell receptor activator is a cross-linker of the B cell receptor complex or the B-cell co-receptor complex. In some embodiments, cross-linker is an antibody or molecular binding entity. In some embodiments, the cross-linker is an antibody. In some embodiments, the antibody is a multivalent antibody. In some embodiments, the antibody is a monovalent, bivalent, or multivalent antibody made more multivalent by attachment to a solid surface or tethered on a nanoparticle surface to increase the local valency of the epitope binding domain.

In some embodiments, the cross-linker is a molecular binding entity. In some embodiments, the molecular binding entity acts upon or binds the B cell receptor complex via carbohydrates or an epitope in the complex. In some embodiments, the molecular is a monovalent, bivalent, or multivalent is made more multivalent by attachment to a solid surface or tethered on a nanoparticle surface to increase the local valency of the epitope binding domain.

In some embodiments, the cross-linking of the B cell receptor complex or the B-cell co-receptor complex comprises binding of an antibody or molecular binding entity to the cell and then causing its crosslinking via interaction of the cell with a solid surface that causes crosslinking of the BCR complex via antibody or molecular binding entity.

In some embodiments, the crosslinker is F(ab)2 IgM, IgG, IgD, polyclonal BCR antibodies, monoclonal BCR antibodies, Fc receptor derived binding elements and/or a combination thereof. The Ig can be derived from a species selected from the group consisting of mouse, goat, rabbit, pig, rat, horse, cow, shark, chicken, or llama. In some embodiments, the crosslinker is F(ab)2 IgM, Polyclonal IgM antibodies, Monoclonal IgM antibodies, Biotinylated F(ab)₂ IgG/M, Biotinylated Polyclonal IgM antibodies, Biotinylated Monoclonal IgM antibodies and/or combination thereof.

In some embodiments, the inhibitor is an inhibitor of a cellular factor or a plurality of factors that participates in a cellular pathway (e.g. signaling cascade) in the cell. In some embodiments, the inhibitor is a kinase or phosphatase inhibitor. Examples of kinase inhibitors are recited above.

In some embodiments H₂O₂ is administered as an inhibitor. In some embodiments H₂O₂ is administered at between 0.01 and 50 mM. In some embodiments H₂O₂ is administered at between 0.1 and 10 mM. In some embodiments H₂O₂ is administered at between 1 and 10 mM. In some embodiments H₂O₂ is administered at between 1 and 5 mM. In some embodiments H₂O₂ is administered at 0.5, 1, 1.5, 2, 2.5, 3, 3.5, 4, 4.5, 5, 5.5, 6, 6.5, 7, 7.5, 8, 8.5, 9, 9.5 or 10 mM. In certain embodiments, H₂O₂ is administered at 3.0 mM. In certain embodiments, H₂O₂ is administered at 3.3 mM. In some embodiments the duration of exposure of H₂O₂ is between 0.01 and 360 minutes. In some embodiments the duration of exposure of H₂O₂ is between 0.1 and 240 minutes. In some embodiments the duration of exposure of H₂O₂ is between 0.5 and 180 minutes. In some embodiments the duration of exposure of H₂O₂ is between 0 and 120 minutes. In some embodiments the duration of exposure to H₂O₂ is between 5 and 15 minutes. In some embodiments the duration of exposure of H₂O₂ is 1, 2, 3, 4, 5, 10, 15, 20, 25, 30, 35, 40, 45, 50, 55, 60, 70, 80, 90, 100, 110, 120, 140, 160 or 180 minutes. In some embodiments the duration of exposure of H₂O₂ is 10 minutes. In some embodiments H₂O₂ is administered as an inhibitor with at least one other modulator. In some embodiments H₂O₂ is administered as an inhibitor with F(ab)2 IgM or any suitable BCR agonist. In some embodiments H₂O₂ is administered before administration of F(ab)₂IgM. In some embodiments H₂O₂ is administered simultaneously with F(ab)2 IgM. In some embodiments H₂O₂ is administered after F(ab)2 IgM.

In some embodiments, the activation level of an activatable element in a cell is determined after contacting the cell with at least 2, 3, 4, 5, 6, 7, 8, 9, or 10 modulators. In some embodiments, the activation level of an activatable element in a cell is determined after contacting the cell with at least 2, 3, 4, 5, 6, 7, 8, 9, or 10 modulators where at least one of the modulators is an inhibitor. In some embodiments, the activation level of an activatable element in a cell is determined after contacting the cell with an inhibitor and a modulator, where the modulator can be an inhibitor or an activator. In some embodiments, the activation level of an activatable element in a cell is determined after contacting the cell with an inhibitor and an activator. In some embodiments, the activation level of an activatable element in a cell is determined after contacting the cell with two or more modulators.

In some embodiments, a phenotypic profile of a population of cells is determined by measuring the activation level of an activatable element when the population of cells is exposed to a plurality of modulators in separate cultures. In some embodiments, the modulators include F(ab)₂ IgM, anti-CD20 antibody (such as rituximab), anti-CD52 antibody (such as Alemtuzumab or Campath), anti-CD22 antibody (such as epratuzumab), anti-CD23 antibody (such as lumiliximab), bendamustine, velcade, H₂O₂, phenylarsine oxide, sodium vanadate, PMA, BAFF, April, SDF1a, CD40L, IGF-1, Imiquimod, polyCpG, fludarabine, cyclophosphamide, chlorambucil, IL-7, IL-6, IL-10, IL-27, IL-4, IL-2, IL-3, thapsigardin and/or a combination thereof. For instance a population of cells can be exposed to one or more, all or a combination of the following combination of modulators: F(ab)2 IgM; anti-CD20 antibody (such as rituximab), anti-CD52 antibody (such as Alemtuzumab or Campath); H₂O₂; PMA; BAFF; April; SDF1a; CD40L; IGF-1; Imiquimod; polyCpG; fludarabine; cyclophosphamide; chlorambucil; IL-7; IL-6; IL-10; IL-27; IL-4; IL-2; IL-3; anti CD22 (epratuzumab), anti-CD23 (lumiliximab), thapsigargin; F(ab)2 IgM; bendamustine; velcade, phenylarsine oxide; and sodium vanadate. In some embodiments, the phenotypic profile of the population of cells is used to classify the population as described herein.

### Detection

In practicing the methods of this invention, the detection of the status of the one or more activatable elements can be carried out by a person, such as a technician in the laboratory. Alternatively, the detection of the status of the one or more activatable elements can be carried out using automated systems. In either case, the detection of the status of the one or more activatable elements for use according to the methods of this invention is performed according to standard techniques and protocols well-established in the art. See U.S. Ser. No. 12/229,976 and 12/460,029 which is incorporated by reference in its entirety.

One or more activatable elements can be detected and/or quantified by any method that can detect and/or quantitate the presence of the activatable element of interest. Such methods may include radioimmunoassay (RIA) or enzyme linked immunoabsorbance assay (ELISA), immunohistochemistry, immunofluorescent histochemistry with or without confocal microscopy, reversed phase assays, homogeneous enzyme immunoassays, and related non-enzymatic techniques, Western blots, whole cell staining, immunoelectronmicroscopy, nucleic acid amplification, gene array, protein array, mass spectrometry, patch clamp, 2-dimensional gel electrophoresis, differential display gel electrophoresis, microsphere-based multiplex protein assays, label-free cellular assays and flow cytometry, etc. U.S. Pat. No. 4,568,649 describes ligand detection systems, which employ scintillation counting. These techniques are particularly useful for modified protein parameters. Cell readouts for proteins and other cell determinants can be obtained using fluorescent or otherwise tagged reporter molecules. Flow cytometry methods are useful for measuring intracellular parameters.

In some embodiments, the present invention provides methods for determining an activatable element's activation profile for a single cell. The methods may comprise analyzing cells by flow cytometry on the basis of the activation level of at least two activatable elements. Binding elements (e.g. activation state-specific antibodies) are used to analyze cells on the basis of activatable element activation level, and can be detected as described below. Alternatively, non-binding elements systems as described above can be used in any system described herein.

When using fluorescent labeled components in the methods and compositions of the present invention, it will recognized that different types of fluorescent monitoring systems, e.g., Cytometric measurement device systems, can be used to practice the invention. In some embodiments, flow cytometric systems are used or systems dedicated to high throughput screening, e.g. 96 well or greater microtiter plates. Methods of performing assays on fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N.J., Modern Molecular Photochemistry, Menlo Park: Benjamin/Cummings Publishing Col, Inc. (1978), pp. 296-361.

Fluorescence in a sample can be measured using a fluorimeter. In general, excitation radiation, from an excitation source having a first wavelength, passes through excitation optics. The excitation optics cause the excitation radiation to excite the sample. In response, fluorescent proteins in the sample emit radiation that has a wavelength that is different from the excitation wavelength. Collection optics then collect the emission from the sample. The device can include a temperature controller to maintain the sample at a specific temperature while it is being scanned. According to one embodiment, a multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation. In general, known robotic systems and components can be used.

Other methods of detecting fluorescence may also be used, e.g., Quantum dot methods (see, e.g., Goldman et al., J. Am. Chem. Soc. (2002) 124:6378-82; Pathak et al. J. Am. Chem. Soc. (2001) 123:4103-4; and Remade et al., Proc. Natl. Sci. USA (2000) 18:553-8, each expressly incorporated herein by reference) as well as confocal microscopy. In general, flow cytometry involves the passage of individual cells through the path of a laser beam. The scattering the beam and excitation of any fluorescent molecules attached to, or found within, the cell is detected by photomultiplier tubes to create a readable output, e.g. size, granularity, or fluorescent intensity.

The detecting, sorting, or isolating step of the methods of the present invention can entail fluorescence-activated cell sorting (FACS) techniques, where FACS is used to select cells from the population containing a particular surface marker, or the selection step can entail the use of magnetically responsive particles as retrievable supports for target cell capture and/or background removal. A variety of FACS systems are known in the art and can be used in the methods of the invention (see e.g., WO99/54494, filed Apr. 16, 1999; U.S. Ser. No. 20010006787, filed Jul. 5, 2001, each expressly incorporated herein by reference).

In some embodiments, a FACS cell sorter (e.g. a FACSVantage™ Cell Sorter, Becton Dickinson Immunocytometry Systems, San Jose, Calif.) is used to sort and collect cells based on their activation profile (positive cells) in the presence or absence of a change in activation level in an activatable element in response to a modulator. In some embodiments the change is a decrease. In some embodiments the change is an increase.

In some embodiments, the cells are first contacted with fluorescent-labeled activation state-specific binding elements (e.g. antibodies) directed against specific activation state of specific activatable elements. In such an embodiment, the amount of bound binding element on each cell can be measured by passing droplets containing the cells through the cell sorter. By imparting an electromagnetic charge to droplets containing the positive cells, the cells can be separated from other cells. The positively selected cells can then be harvested in sterile collection vessels. These cell-sorting procedures are described in detail, for example, in the FACSVantage™. Training Manual, with particular reference to sections 3-11 to 3-28 and 10⁻¹ to 10-17, which is hereby incorporated by reference in its entirety.

In another embodiment, positive cells can be sorted using magnetic separation of cells based on the presence of an isoform of an activatable element. In such separation techniques, cells to be positively selected are first contacted with specific binding element (e.g., an antibody or reagent that binds an isoform of an activatable element). The cells are then contacted with retrievable particles (e.g., magnetically responsive particles) that are coupled with a reagent that binds the specific element. The cell-binding element-particle complex can then be physically separated from non-positive or non-labeled cells, for example, using a magnetic field. When using magnetically responsive particles, the positive or labeled cells can be retained in a container using a magnetic filed while the negative cells are removed. These and similar separation procedures are described, for example, in the Baxter Immunotherapy Isolex training manual which is hereby incorporated in its entirety.

In some embodiments, methods for the determination of a receptor element activation state profile for a single cell are provided. The methods comprise providing a population of cells and analyze the population of cells by flow cytometry. Preferably, cells are analyzed on the basis of the activation level of at least two activatable elements. In some embodiments, a multiplicity of activatable element activation-state antibodies is used to simultaneously determine the activation level of a multiplicity of elements.

In some embodiment, cell analysis by flow cytometry on the basis of the activation level of at least two elements is combined with a determination of other flow cytometry readable outputs, such as the presence of surface markers, granularity and cell size to provide a correlation between the activation level of a multiplicity of elements and other cell qualities measurable by flow cytometry for single cells.

As will be appreciated, the present invention also provides for the ordering of element clustering events in signal transduction. Particularly, the present invention allows the artisan to construct an element clustering and activation hierarchy based on the correlation of levels of clustering and activation of a multiplicity of elements within single cells. Ordering can be accomplished by comparing the activation level of a cell or cell population with a control at a single time point, or by comparing cells at multiple time points to observe subpopulations arising out of the others.

The present invention provides a valuable method of determining the presence of cellular subsets within cellular populations. Ideally, signal transduction pathways are evaluated in homogeneous cell populations to ensure that variances in signaling between cells do not qualitatively nor quantitatively mask signal transduction events and alterations therein. As the ultimate homogeneous system is the single cell, the present invention allows the individual evaluation of cells to allow true differences to be identified in a significant way.

Thus, the invention provides methods of distinguishing cellular subsets within a larger cellular population. As outlined herein, these cellular subsets often exhibit altered biological characteristics (e.g. activation levels, altered response to modulators) as compared to other subsets within the population. For example, as outlined herein, the methods of the invention allow the identification of subsets of cells from a population such as primary cell populations, e.g. peripheral blood mononuclear cells that exhibit altered responses (e.g. response associated with presence of a condition) as compared to other subsets. In addition, this type of evaluation distinguishes between different activation states, altered responses to modulators, cell lineages, cell differentiation states, etc.

As will be appreciated, these methods provide for the identification of distinct signaling cascades for both artificial and stimulatory conditions in complex cell populations, such a peripheral blood mononuclear cells, or naive and memory lymphocytes.

When necessary, cells are dispersed into a single cell suspension (e.g. by enzymatic digestion with a suitable protease, collagenase, dispase, etc; and the like). An appropriate solution is used for dispersion or suspension. Such solution will generally be a balanced salt solution, e.g. normal saline, PBS, Hanks balanced salt solution, etc., conveniently supplemented with fetal calf serum or other naturally occurring factors, in conjunction with an acceptable buffer at low concentration, generally from 5-25 mM. Convenient buffers include HEPES1 phosphate buffers, lactate buffers, etc. The cells may be fixed, e.g. with 3% paraformaldehyde, and are usually permeabilized, e.g. with ice cold methanol; HEPES-buffered PBS containing 0.1% saponin, 3% BSA; covering for 2 min in acetone at −200 C; and the like as known in the art and according to the methods described herein.

In some embodiments, one or more cells are contained in a well of a 96 well plate or other commercially available multi-well plate. In an alternate embodiment, the reaction mixture or cells are in a cytometric measurement device. Other multi-well plates useful in the present invention include, but are not limited to 384 well plates and 1536 well plates. Still other vessels for containing the reaction mixture or cells and useful in the present invention will be apparent to the skilled artisan.

The addition of the components of the assay for detecting the activation level or activity of an activatable element, or modulation of such activation level or activity, may be sequential or in a predetermined order or grouping under conditions appropriate for the activity that is assayed for. Such conditions are described here and known in the art. Moreover, further guidance is provided below (see, e.g., in the Examples).

In some embodiments, the activation level of an activatable element is measured using Inductively Coupled Plasma Mass Spectrometer (ICP-MS). A binding element that has been labeled with a specific element binds to the activatable element. When the cell is introduced into the ICP, it is atomized and ionized. The elemental composition of the cell, including the labeled binding element that is bound to the activatable element, is measured. The presence and intensity of the signals corresponding to the labels on the binding element indicates the level of the activatable element on that cell (Tanner et al. Spectrochimica Acta Part B: Atomic Spectroscopy, (2007), 62(3):188-195.).

As will be appreciated by one of skill in the art, the instant methods and compositions find use in a variety of other assay formats in addition to flow cytometry analysis. For example, a chip analogous to a DNA chip can be used in the methods of the present invention. Arrayers and methods for spotting nucleic acid to a chip in a prefigured array are known. In addition, protein chips and methods for synthesis are known. These methods and materials may be adapted for the purpose of affixing activation state binding elements to a chip in a prefigured array. In some embodiments, such a chip comprises a multiplicity of element activation state binding elements, and is used to determine an element activation state profile for elements present on the surface of a cell.

In some embodiments, a chip comprises a multiplicity of the “second set binding elements,” in this case generally unlabeled. Such a chip is contacted with sample, preferably cell extract, and a second multiplicity of binding elements comprising element activation state specific binding elements is used in the sandwich assay to simultaneously determine the presence of a multiplicity of activated elements in sample. Preferably, each of the multiplicity of activation state-specific binding elements is uniquely labeled to facilitate detection.

In some embodiments confocal microscopy can be used to detect activation profiles for individual cells. Confocal microscopy relies on the serial collection of light from spatially filtered individual specimen points, which is then electronically processed to render a magnified image of the specimen. The signal processing involved confocal microscopy has the additional capability of detecting labeled binding elements within single cells, accordingly in this embodiment the cells can be labeled with one or more binding elements. In some embodiments the binding elements used in connection with confocal microscopy are antibodies conjugated to fluorescent labels, however other binding elements, such as other proteins or nucleic acids are also possible.

In some embodiments, the methods and compositions of the instant invention can be used in conjunction with an “In-Cell Western Assay.” In such an assay, cells are initially grown in standard tissue culture flasks using standard tissue culture techniques. Once grown to optimum confluency, the growth media is removed and cells are washed and trypsinized. The cells can then be counted and volumes sufficient to transfer the appropriate number of cells are aliquoted into microwell plates (e.g., Nunc™ 96 Microwell™ plates). The individual wells are then grown to optimum confluency in complete media whereupon the media is replaced with serum-free media. At this point controls are untouched, but experimental wells are incubated with a modulator, e.g. EGF. After incubation with the modulator cells are fixed and stained with labeled antibodies to the activation elements being investigated. Once the cells are labeled, the plates can be scanned using an imager such as the Odyssey Imager (LiCor, Lincoln Nebr.) using techniques described in the Odyssey Operator's Manual v1.2, which is hereby incorporated in its entirety. Data obtained by scanning of the multi-well plate can be analyzed and activation profiles determined as described below.

In some embodiments, the detecting is by high pressure liquid chromatography (HPLC), for example, reverse phase HPLC, and in a further aspect, the detecting is by mass spectrometry.

These instruments can fit in a sterile laminar flow or fume hood, or are enclosed, self-contained systems, for cell culture growth and transformation in multi-well plates or tubes and for hazardous operations. The living cells may be grown under controlled growth conditions, with controls for temperature, humidity, and gas for time series of the live cell assays. Automated transformation of cells and automated colony pickers may facilitate rapid screening of desired cells.

Flow cytometry or capillary electrophoresis formats can be used for individual capture of magnetic and other beads, particles, cells, and organisms.

Flexible hardware and software allow instrument adaptability for multiple applications. The software program modules allow creation, modification, and running of methods. The system diagnostic modules allow instrument alignment, correct connections, and motor operations. Customized tools, labware, and liquid, particle, cell and organism transfer patterns allow different applications to be performed. Databases allow method and parameter storage. Robotic and computer interfaces allow communication between instruments.

In some embodiment, the methods of the invention include the use of liquid handling components. The liquid handling systems can include robotic systems comprising any number of components. In addition, any or all of the steps outlined herein may be automated; thus, for example, the systems may be completely or partially automated.

As will be appreciated by those in the art, there are a wide variety of components which can be used, including, but not limited to, one or more robotic arms; plate handlers for the positioning of microplates; automated lid or cap handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation. Additional examples of automation, automated sample collection and analysis are disclosed in U.S. 61/048,657 which is hereby incorporated by reference in its entirety.

In some embodiments, chemically derivatized particles, plates, cartridges, tubes, magnetic particles, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In some embodiments, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradeable modular platform for additional capacity. This modular platform includes a variable speed orbital shaker, and multi-position work decks for source samples, sample and reagent dilution, assay plates, sample and reagent reservoirs, pipette tips, and an active wash station. In some embodiments, the methods of the invention include the use of a plate reader.

In some embodiments, thermocycler and thermoregulating systems are used for stabilizing the temperature of heat exchangers such as controlled blocks or platforms to provide accurate temperature control of incubating samples from 0° C. to 100° C.

In some embodiments, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some embodiments, the instrumentation will include a detector, which can be a wide variety of different detectors, depending on the labels and assay. In some embodiments, useful detectors include a microscope(s) with multiple channels of fluorescence; plate readers to provide fluorescent, ultraviolet and visible spectrophotometric detection with single and dual wavelength endpoint and kinetics capability, fluorescence resonance energy transfer (FRET), luminescence, quenching, two-photon excitation, and intensity redistribution; CCD cameras to capture and transform data and images into quantifiable formats; and a computer workstation.

In some embodiments, the robotic apparatus includes a central processing unit which communicates with a memory and a set of input/output devices (e.g., keyboard, mouse, monitor, printer, etc.) through a bus. Again, as outlined below, this may be in addition to or in place of the CPU for the multiplexing devices of the invention. The general interaction between a central processing unit, a memory, input/output devices, and a bus is known in the art. Thus, a variety of different procedures, depending on the experiments to be run, are stored in the CPU memory.

These robotic fluid handling systems can utilize any number of different reagents, including buffers, reagents, samples, washes, assay components such as label probes, etc.

### Analysis

Advances in flow cytometry have enabled the individual cell enumeration of up to thirteen simultaneous parameters (De Rosa et al., 2001) and are moving towards the study of genomic and proteomic data subsets (Krutzik and Nolan, 2003; Perez and Nolan, 2002). Likewise, advances in other techniques (e.g. microarrays) allow for the identification of multiple activatable elements. As the number of parameters, epitopes, and samples have increased, the complexity of experiments and the challenges of data analysis have grown rapidly. An additional layer of data complexity has been added by the development of stimulation panels which enable the study of activatable elements under a growing set of experimental conditions. Methods for the analysis of multiple parameters are well known in the art. In some embodiments flow cytometry applications require software for different phases of operation and analysis, see 61/079,579; 61/079,551; 61/079,537; 61/087,555; 61/085,789 which are hereby incorporated by reference in their entireties.

In some embodiments where flow cytometry is used, flow cytometry experiments are arrayed and the results are approximated as fold changes using a heat map to facilitate evaluation. Generally speaking, arrayed flow cytometry experiments simplify multidimensional flow cytometry data based on experimental design and observed differences between flow cytometry samples. One common way of comparing changes in a set of flow cytometry samples is to overlay histograms of one parameter on the same plot. Arrayed flow cytometry experiments ideally contain a reference sample against which experimental samples are compared. This reference sample is placed in the first position of the array, and subsequent experimental samples follow the control in the sequence. Reference samples can include normal and/or cells associated with a condition (e.g. tumor cells).

In some embodiments where flow cytometry is used, prior to analyzing of data the populations of interest and the method for characterizing these populations are determined. For instance, there are at least two general ways of identifying populations for data analysis: (i) “Outside-in” comparison of Parameter sets for individual samples or subset (e.g., patients in a trial). In this more common case, cell populations are homogenous or lineage gated in such a way as to create distinct sets considered to be homogenous for targets of interest. An example of sample-level comparison would be the identification of signaling profiles in tumor cells of a patient and correlation of these profiles with non-random distribution of clinical responses. This is considered an outside-in approach because the population of interest is pre-defined prior to the mapping and comparison of its profile to other populations. (ii) “Inside-out” comparison of Parameters at the level of individual cells in a heterogeneous population. An example of this would be the signal transduction state mapping of mixed hematopoietic cells under certain conditions and subsequent comparison of computationally identified cell clusters with lineage specific markers. This could be considered an inside-out approach to single cell studies as it does not presume the existence of specific populations prior to classification. A major drawback of this approach is that it creates populations which, at least initially, require multiple transient markers to enumerate and may never be accessible with a single cell surface epitope. As a result, the biological significance of such populations can be difficult to determine. The main advantage of this unconventional approach is the unbiased tracking of cell populations without drawing potentially arbitrary distinctions between lineages or cell types.

Each of these techniques capitalizes on the ability of flow cytometry to deliver large amounts of multiparameter data at the single cell level. For cells associated with a condition (e.g. neoplastic, autoimmune or hematopoetic condition), a third “meta-level” of data exists because cells associated with a condition (e.g. cancer cells) are generally treated as a single entity and classified according to historical techniques. These techniques have included organ or tissue of origin, degree of differentiation, proliferation index, metastatic spread, and genetic or metabolic data regarding the patient.

In some embodiments, the present invention uses variance mapping techniques for mapping condition signaling space. These methods represent a significant advance in the study of condition biology because it enables comparison of conditions independent of a putative normal control. Traditional differential state analysis methods (e.g., DNA microarrays, subtractive Northern blotting) generally rely on the comparison of cells associated with a condition from each patient sample with a normal control, generally adjacent and theoretically untransformed tissue. Alternatively, they rely on multiple clusterings and re-clusterings to group and then further stratify patient samples according to phenotype. In contrast, variance mapping of condition states compares condition samples first with themselves and then against the parent condition population. As a result, activation states with the most diversity among conditions provide the core parameters in the differential state analysis. Given a pool of diverse conditions, this technique allows a researcher to identify the molecular events that underlie differential condition pathology (e.g., cancer responses to chemotherapy), as opposed to differences between conditions and a proposed normal control.

In some embodiments, when variance mapping is used to profile the signaling space of patient samples, conditions whose signaling response to modulators is similar are grouped together, regardless of tissue or cell type of origin. Similarly, two conditions (e.g. two tumors) that are thought to be relatively alike based on lineage markers or tissue of origin could have vastly different abilities to interpret environmental stimuli and would be profiled in two different groups.

When groups of signaling profiles have been identified it is frequently useful to determine whether other factors, such as clinical responses, presence of gene mutations, and protein expression levels, are non-randomly distributed within the groups. If experiments or literature suggest such a hypothesis in an arrayed flow cytometry experiment, it can be judged with simple statistical tests, such as the Student's t-test and the X² test. Similarly, if two variable factors within the experiment are thought to be related, the r² correlation coefficient from a linear regression is used to represent the degree of this relationship.

In one embodiment of the invention, several metrics have been developed that compare distributions of per cell fluorescent intensities. These metrics may operate with one or more readouts (i.e., in one or more dimensions). The metrics may compare fluorescent intensities between cells (possibly from gated populations) from a single sample in basal and modulated states or compare the fluorescent intensities of cells from a given sample to a reference distribution of intensities; the fluorescent intensities may be untransformed compensated data or transformed using functions such as logarithm of base 2, natural logarithm, logarithm of base 10, arcsinh, etc. The reference distribution may be derived from a cohort of samples in a current experiment or from historical data and may comprise cells in one or more states including basal and modulated with one or more modulators; in addition, the reference distribution may be from a gated population, which may or may not be the same population as the population for which the metric is calculated (e.g., the metric may be computed comparing B-cells to T-cells). The reference distribution may be treated as discrete cell events, as a histogram of cell events (representing frequencies of intensities) or as one of a plurality of distribution functions (e.g., normal, beta, gamma, exponential, Dirichlet, non-uniform rational B-splines, etc.) The parameters for the distribution functions describing the distributions of cell events may be derived via methods including expectation maximization [REF: Hastie, Tibshirani, and Friedman, The Elements of Statistical Learning, pp 236-243, 2001.], Markov-chain Monte Carlo, or spectral methods[e.g., FFT]. Comparison of curves may be performed using a variety of metrics including Area Under the Curve (AUC), Cohen's D (Jacob Cohen (1988), Statistical Power Analysis for the Behavioral Sciences (second ed.)), Chi-Square, Kolmogorov-Smirnov, or other statistics.

One embodiment of the invention uses Uᵤ. This metric is designed to estimate the overlap between one and multi-dimensional distributions of cells that have been treated with a modulator and those that have not been treated with a modulator. Cells from both the modualted and unmodulated wells are ranked in decreasing order of intensity values for an antibody-fluorochrome conjugate. These rankings are then converted to an Receiver operating characteristic (ROC) curve, with the fraction of unmodulated cells on the x-axis and the fraction modualted cells on the y-axis. As one moves down the ranked list, an empirical ROC curve can be plotted by either moving parallel to the y-axis by 1/N_{modulated} if one encounters a modulated cell or the x-axis by 1N^{unmodulated} if one encounters a unmodulated cell. The Uᵤ metric is then computed as a area under the ROC curve. The Uu metric may also be considered as the scaled Mann-Whitney U statistic. If one encounters only modulated cells before any unmodulated cells, the Uᵤ metric will equal 1.0. One the other hand, if all modulated cells are ranked lower than the modulated cells, AUCᵤₛ will equal 0.0. Finally, a perfect overlap between the two distributions, with the chance of encounter a modulated or unmodulated cells at a given intensity is about the same, Uᵤ will be close to 0.5.

Examples of analysis for activatable elements are described in US publication number 20060073474 entitled “Methods and compositions for detecting the activation state of multiple proteins in single cells” and US publication number 20050112700 entitled “Methods and compositions for risk stratification” and U.S. Ser. Nos. 61/085,789 and 12/229,976, which are hereby incorporated by reference in their entirety.

CLL serves as an example of the methods of the invention. The data shown in FIGS. 26, 27 and 28 of U.S. Ser. No. 12/229,976 (976) is a heat map comparing the activation states of multiple activatable elements in 22 CLL patients and 4 control patients. This data demonstrates that B-cells from various CLL patients display distinguishable patterns of activatable elements as visualized by a heat map. An inhibitor or inhibitor plus another modulator further define additional patterns of activatable elements that allow identification, classification and grouping of cryptic or aberrant hematopoietic populations (i.e. patient clustering). In FIGS. 26, 27 & 28 patient samples are indicated at the top of the heat map. Each column represents a single patient. CLL indicates that the sample was obtained from a patient diagnosed with CLL. CON indicates that the sample was obtained from a control patient. The heat map legend is indicated at the top of the figure and uses a shaded scale based on the log 10-fold increase, or decrease, in mean fluorescence intensity (MFI), relative to the unstimulated control (0 min).

The heat map depicts the activation state of various activatable elements by denoting a change, or lack thereof, in the level of an activatable element revealed by the presence of an inhibitor and/or additional modulator. Thus, the heat map can depict the presence or absence of an increase in the activation level of a plurality of activatable elements in a cell upon contacting said cell with an inhibitor or a modulator. Labels to the right of the heat map indicate the activatable element detected, e.g. a phospho-protein. Labels to the right also indicate the modulator or inhibitor treatment for that row. “US” indicates unstimulated or untreated. FIG. 28 of '976 illustrates a pattern of activation levels of a plurality of activatable elements in a cell. FIG. 28 further illustrates the identification of patient clustering groups (i.e. clustering groups). A patient clustering group is comprised of samples from patients that display similar or distinct patterns of activation levels in one or more activatable elements in response to one or more modulators (e.g., an inhibitor, or an inhibitor and another modulator). FIG. 28 of '976 illustrates a clustering group comprised of samples from patients in which the activation levels of p-PLCγ2, p-SyK/ZAP-70, p-BLNK and p-Lyn are similar in response to the same stimulus. Some patient clustering groups are revealed upon modulation or treatment with an inhibitor as illustrated by the boxed regions. Treatment with H₂O₂ reveals a patient clustering group defined by the levels of p-PLCγ2, p-Syk/ZAP-70, p-BLNK and p-Lyn (FIG. 28, bottom right boxed area) that are similar to those of the four control patients (FIG. 28, bottom center box). Treatment with H₂O₂ further reveals a patient clustering group that is distinct from the controls (FIG. 28, 9 patients to the left of bottom boxed area). Modulation with H₂O₂ and BCR crosslinking defines another patient clustering group comprised of samples from patients that display the activation levels of p-BLNK, p-Syk and p-PLCγ2 (FIG. 28, top left boxed area) that are similar to the control patients (top center box). In addition, modulation with H₂O₂ and BCR crosslinking further reveals another clustering group distinct from the controls (10 patients to the right of top boxed area).

Thus, also provided herein is a method of deriving a classification. Deriving a classification involves defining a clustering group. A clustering group is defined by determining the activation state of a plurality of activatable elements from a plurality of cells wherein each cell is derived from an individual with a known conditions and/or known clinical outcome. A clustering group may define a pattern that associated with a known condition or known clinical outcome. Any suitable activatable element can be used wherein the activation level of said activatable element provides useful information regarding a known condition or clinical outcome of a patient. A cell derived from a patient with an unknown condition and/or unknown clinical outcome may be classified depending upon which clustering group it is identified with. This can further lead to diagnosis, prognosis, and/or evaluation or choice of treatment for the patient.

In another embodiment, measurements of expression or induced change in expression combinations of two activatable elements treated with one or more modulators may be used as inputs to algorithms such as logistic regression modeling or generally known classification methods to produce a score. The score may, for example, indicate the likelihood of response to fludarabine. Examples of modulator and activatable element (written as modulator→activatable element) combinations are: H₂O₂→p-Erk+anti-μ or anti-γ→p-STAT5, anti-μ or anti-γ→p-STAT5+H₂O₂→p-S6, H₂O₂→p-Lyn+Fludarabine→Caspase8, H₂O₂→p-PLCγ2+Unstim→CD5, H₂O₂→_{P}-65-RelA+Unstim→CD5, H₂O₂→p-Erk+Unstim→CD5, Fludarabine→Caspase8+H₂O₂→p-S6, H₂O₂→p-BLNK+Fludarabine→Caspase8, H₂O₂→p-Lyn+Unstim→CD5, H₂O₂→p-Syk+Fludarabine→Cytochrome-C, H₂O₂→p-S6+Unstim→CD5, Unstim→SHP2+Fludarabine→Caspase8, H₂O₂→p-PLCγ2+Fludarabine→Caspase8, H₂O₂→p-Syk+Fludarabine→Caspase8, H₂O₂→p-BLNK+Unstim→CD5, H₂O₂→p-Lyn+Fludarabine→Cytochrome-C, Fludarabine→Cytochrome-C+H₂O₂→p-STAT5, H₂O₂→p-Syk+Unstim→CD5, H₂O₂→p-STAT5+Unstim→CD5, Unstim→CD20+Fludarabine→Caspase8, Staurosporine→Cytochrome-C+H₂O₂→p-65—RelA, H₂O₂→p-BLNK+Fludarabine→Cytochrome-C, Fludarabine→Caspase8+H₂O₂→p-Erk, H₂O₂→p-Erk+Fludarabine→Cytochrome-C, H₂O₂→P-S6+Staurosporine→Cytochrome-C, H₂O₂→p-PLCγ2+Fludarabine→Cytochrome-C, Staurosporine→Cytochrome-C+Unstim→IgM, H₂O₂→p-Syk+Unstim→IgM, H₂O₂→p-STAT5+Unstim→CD22, Unstim→CD5+Unstim→CD38, Unstim→CD20+Unstim→CD5, anti-μ or anti-Γ→p-STAT5+Unstim→CD38, H₂O₂→p-Lyn+Unstim→CD22, Unstim→SHP2+Unstim→CD5, Unstim→CD20+ anti-μ or anti-γ→p-PLCγ2.

### Kits

In some embodiments the invention provides kits. Kits provided by the invention may comprise one or more of the state-specific binding element described herein, such as phospho-specific antibodies. In some embodiments, the kit comprises one or more of the phospho-specific antibodies specific for the proteins selected from the group consisting of PI3-Kinase (p85, p110a, p110b, p110d), Jak1, Jak2, SOCs, Rac, Rho, Cdc42, Ras-GAP, Vav, Tiam, Sos, Dbl, Nck, Gab, PRK, SHPT, and SHP2, SHIP1, SHIP2, sSHIP, PTEN, Shc, Grb2, PDK1, SGK, Akt1, Akt2, Akt3, TSC1,2, Rheb, mTor, 4EBP-1, p70S6Kinase, S6, LKB-1, AMPK, PFK, Acetyl-CoAa Carboxylase, DokS, Rafs, Mos, Tp12, MEK1/2, MLK3, TAK, DLK, MKK3/6, MEKK1,4, MLK3, ASK1, MKK4/7, SAPK/JNK1,2,3, p38s, Erk1/2, Syk, Btk, BLNK, LAT, ZAP-70, Lyn, Cbl, SLP-76, PLCγ₁, PLCγ2, STAT1, STAT2, STAT3, STAT4, STAT5, STAT6, FAK, p130CAS, PAKs, LIMK1/2, Hsp90, Hsp70, Hsp27, SMADs, Rel-A (p65-NFKB), CREB, Histone H₂B, HATs, HDACs, PKR, Rb, Cyclin D, Cyclin E, Cyclin A, Cyclin B, P16, p14Arf, p27KIP, p21CIP, Cdk4, Cdk6, Cdk7, Cdk1, Cdk2, Cdk9, Cdc25, A/B/C, Abl, E2F, FADD, TRADD, TRAF2, RIP, Myd8 8, BAD, Bcl-2, Mcl-1, Bcl-XL, Caspase 2, Caspase 3, Caspase 6, Caspase 7, Caspase 8, Caspase 9, PARP, IAPB, Smac, Fodrin, Actin, Src, Lyn, Fyn, Lyn, NIK, IκB, p65(RelA), IKKα, PKA, PKCα, PKCβ, PKCθ, PKCδ, CAMK, Elk, AFT, Myc, Egr-1, NFAT, ATF-2, Mdm2, p53, DNA-PK, Chk1, Chk2, ATM, ATR, \tilde{β}catenin, CrkL, GSK3α, GSK3β, and FOXO. In some embodiments, the kit comprises one or more of the phospho-specific antibodies specific for the proteins selected from the group consisting of Erk, Syk, ZAP-70, Lyn, Btk, BLNK, Cbl, PLCγ2, Akt, RelA, p38, S6. In some embodiments, the kit comprises one or more of the phospho-specific antibodies specific for the proteins selected from the group consisting of Akt1, Akt2, Akt3, SAPK/JNK1,2,3, p38s, Erk1/2, Syk, ZAP-70, Btk, BLNK, Lyn, PLCγ, PLC?y 2, STAT1, STAT3, STAT4, STAT5, STAT6, CREB, Lyn, p-S6, Cbl, NF-κB, GSK3β, CARMA/Bcl10 and Tcl-1.

Kits provided by the invention may comprise one or more of the modulators described herein. In some embodiments, the kit comprises one or more modulators selected from the group consisting of F(ab)2 IgM, H₂O₂, PMA, BAFF, April, SDF1a, CD40L, IGF-1, Imiquimod, polyCpG, IL-7, IL-6, IL-10, IL-27, IL-4, IL-2, IL-3, thapsigargin and a combination thereof. One embodiment is a kit containing H₂O₂ and a drug of interest, such as fludaribine (F-ara-A) and optionally anti-g as modulators, and detection elements, such as antibodies, directed to CD3, CD5, CD20 for external cell surface markers, as well as p-STAT5, p-PLCγ, cleaved Caspase 3 and cleaved PARP. Optionally, controls such as Ramos cells or peripheral blood mononuclear cells (PBMCs) from healthy donors can be included in the kit. These cells may be fresh, frozen, lyophilized or in any other appropriate state. In one embodiment, the kit comprises modulators such as H₂O₂ and anti-g, as well as detection elements directed to one or more of the following: p-Lyn, p-Syk, p-BLNK, p-PLCγ2, p-Erk, p-Akt, p-S6, p-65/RelA, as well as non-canonical signaling markers such as p-STAT5. Inclusion of fludaribine into a kit will be useful to analyze cell responses to that drug.

The state-specific binding element of the invention can be conjugated to a solid support and to detectable groups directly or indirectly. The reagents may also include ancillary agents such as buffering agents and stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The kit may be packaged in any suitable manner, typically with all elements in a single container along with a sheet of printed instructions for carrying out the test.

Such kits enable the detection of activatable elements by sensitive cellular assay methods, such as IHC and flow cytometry, which are suitable for the clinical detection, prognosis, and screening of cells and tissue from patients, such as leukemia patients, having a disease involving altered pathway signaling.

Such kits may additionally comprise one or more therapeutic agents. The kit may further comprise a software package for data analysis of the physiological status, which may include reference profiles for comparison with the test profile.

Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such kits may also include instructions to access a database such as described in U.S. Ser. No. 61/087,555 for selecting an antibody specific for the pathway of interest. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are expressly incorporated by reference in their entirety.

### EXAMPLES

### Example 1

### Signaling Pathways in CLL Samples

### Cell Preparation

Intracellular network responses of CLL patient samples subjected to modulators of signaling, were analyzed using flow cytometry-based Single Cell Network Profiling (SCNP). Of the many signaling modulators studied, H₂O₂ treatment (a general inhibitor of tyrosine phosphatase activity) stratified. CLL patients into two subsets, one showing augmented. BCR signaling and the second with little or no response. These data suggest that differential phosphatase activity with consequent aberrations in tonic (ligand independent) signaling proximal to BCR signaling was driving the biology of these two patient groups. Importantly, signaling in patients was reflected in all the measured components of the canonical B cell receptor network. Thus, p-Lyn, p-Syk, p-BLNK, p-PLCγ2, p-Erk and p-Akt showed parallel phosphorylation responses and were either augmented in unison, or not activated at all. In vitro F-Ara-A—exposure of samples from the same group of CLL patients identified patients with a significant number of apoptosis competent cells, and other patients that were refractory to apoptotic induction in vitro. Statistical analysis of the two data sets revealed that the capacity of patient samples to show peroxide-mediated augmented BCR signaling was highly associated with the ability of cells in these patients to exhibit apoptotic proficiency to F-Ara-A in vitro. This potential link between mechanisms governing apoptosis, phosphatase activity and BCR signaling in B cells provides a means of identifying patient samples that are either responsive or refractory to a given therapy. By extrapolation, such a test based on these separation criteria could play an informative role in the choice of therapeutic agent for patients.

Cryopreserved PBMCs, 23 from CLL patients and 7 from healthy donors, were rapidly thawed in a 37° C. water bath. 1 mL of pre-warmed thawing media (PBS 1% FBS, 2 mM EDTA) was added dropwise to each of the cryovials. Thawed cells were transferred to a tube containing 8 mL thawing media. Tubes were inverted and centrifuged at 200×g for 8 minutes at room temperature. Supernatant was decanted, cell pellets were resuspended in 1 mL RPMI 1640 1% FCS and filtered over 70 um nylon mesh (BD Falcon) to remove cell clumps and debris. 11 mL of additional RPMI 1640 1% FCS was added to the samples to wash. A 20 uL aliquot was removed from each sample and placed into a solution of PBS 4% FCS CD45 Alexa Flour 700 and Propidium Iodide for viability and counting on a BDLSRII cytometer. The remaining volume of cell suspension was centrifuged at 200×g for 8 minutes at room temperature

Supernatant was aspirated and each sample was resuspended in 4 mL of PBS. 4 mL of 2× Amine Aqua (Invitrogen) was added to each sample and incubated at 37° C. for 15 minutes. 10 mL of RPMI 1640 1% FCS was added to each sample to neutralize the Amine Aqua staining and samples were centrifuged at 200×g for 8 minutes at room temperature. The supernatant was decanted and all samples were resuspended at a density of 2.4×10⁶ cells/mL, using a volume specific to the number of cells determined from the cell counting procedure. Samples were arrayed in a 96-well deep-well “mother” plate according to donor ID.

“Daughter” plates designated for treatment with modulators for or apoptosis inducing agents were generated from the “mother” plate with the use of a Liquidator 96-well pipettor (Rainin) Plates designated for treatment with modulators for phospho-readouts received 250 uL (6.0×10⁵ cells) of cell suspension per well, and plates designated for treatment with apoptosis inducing agents received 333 uL (8.0×10⁵ cells) of cell suspension per well. The “daughter” plates were prepared in duplicate and allowed to rest for 1 hour in an incubator at 37° C., 5% CO₂ before treatment.

### Cell Counting

A 20 uL cell suspension from each sample was incubated in individual wells of a 96-well u-bottom plate (BD Falcon) in 180 uL of PBS 4% FCS, CD45 Alexa Fluor700, and 1 μg/mL Propidium Iodide for 10 minutes at room temperature, shielded from light. After 10 minutes 254 of each sample was run on a BDLSRII cytometer (BDIS, San Jose, Calif.) equipped with a high throughput sampler (HTS). Events were gated on CD45+, PI−. Counts of events in the CD45+, PI-gate were exported in a CSV file and total cell numbers were calculated in Microsoft Excel.

### Ramos Cell Line Control

Ramos cell line controls were acquired from ATCC and cultured according to the manufacturer's protocol.

### Phenotypic Staining on Unfixed PBMCs

Three panels of fluorochrome conjugated antibodies were incubated with each sample. All three panels contained four commom mAbs: CD3 Pacific Blue, CD20 PerCP Cy5.5, CD5 biotin, and CD19 Alexa Flour 700. The varying combinations of mAbs in each of the panels was listed: Panel 1: IgM FITC, IgD PE, IgG APC; Panel 2: λ-light chain FITC, κ-light chain PE, CD38 APC; Panel 3: CD45 FITC, CD7913 PE, CD22 APC.

All antibodies for each panel were cocktailed in pre-titered saturating concentrations. 504 of each cocktail were aliquoted and arrayed in a deep-well 96-well plate. 42 μL of each sample from the prepared “mother” plate was added to the wells containing antibody cocktail. Cells were incubated for 30 minutes at room temperature, shielded from light.

After 30 minutes the cells were washed with 1 mL PBS and centrifuged at 400×g. After centrifugation the supernatant was aspirated. The 2° staining cocktail was prepared by adding 0.254, of streptavidin Qdot605 (Invitrogen) to 9.754 of PBS. 10 μL of 2° staining cocktail was added to each sample and incubated for 30 minutes at room temperature, shielded from light.

After 30 minutes the cells were washed with 1 mL FACS Buffer (PBS, 0.5% BSA, 0.05% NaN₃) and centrifuged at 400×g. After centrifugation the supernatant was aspirated and 1004 of FACS Buffer was added to each well. The total volume of each well was transferred to a standard depth u-bottom 96-well plate (BD Falcon) for acquisition on a BD FACSCantoII equipped with the HTS unit.

### Modulation of Cells

Each sample was treated in bulk for 10 minutes at 37° C. with goat polyclonal IgM or IgG (F(ab′)₂, (Southern Biotech), final concentration 10 μg/ml, Phorbol Myristate Acetate ((PMA) Sigma), final concentration 400 nM, and H₂O₂, final concentration 3.3 mM. For the combination of anti-g and H₂O₂, anti-μwas added first followed by H₂O₂ within 30 seconds.

For single agent modulators 150 μL of 10× solutions were arrayed into wells of a 96-well v-bottom plate (Nunc). Corresponding daughter plates were taken from the 37° C. incubator, and 200 mL of RPMI1640 1% FCS was added to each well using the Liquidator 96-well pipettor. Using a Hydra, 50 μL of 10× modulator solution was aspirated from the 10× plate and delivered to the daughter plates. The daughter plates were pulse vortexed for 5 seconds and placed into a 37° C. water bath for 10 minutes. At the end of the 10 minute incubation 2004, 5.6% PFA (final 1.6%) was added to each well to fix the cells.

For BCR x-linking in combination with phosphatase inhibitor H₂O₂, a “BCR α-link” 96-well deep-well plate was loaded with 500 μL of RPMI1640 1% FCS or 25 μg/mL IgM F(ab′)₂ in the appropriate wells. Another “H₂O₂” standard 96-well v-bottom plate was prepared with RPMI1640 1% FCS or 33 mM H₂O₂. Corresponding daughter plates were taken from the 37° C. incubator. Using the Liquidator 96-well pipettor (Rainin) 200 uL was aspirated from the “BCR X-link” plate and added to the daughter plates with cells. Not allowing more than 30 seconds to pass, 50 uL of 10×H₂O₂ was aspirated from the “H₂O₂” plate and added to the daughter plate using the Hydra 96-well pipettor (Matrix). The plate was pulse vortexed for 5 seconds and incubated in a 37° C. water bath for ten minutes. At the end of the 10 minute incubation 200 uL 5.6% PFA (final 1.6%) was added to each well to fix the cells.

After 10 minute incubation at 37° C. all plates were centrifuged at 1000×g for 5 minutes at room temperature. Supernatant was aspirated and plates were vortexed for 30 seconds to disrupt cell pellet. Cells were permeabilized by adding 600 μL of ice cold 100% methanol to each well using the Costar 96-channel pipettor (Costar). Plates were covered with adherent foil seals and placed in a −80° C. freezer for at least 24 hours.

### Treatment of Cells With Apoptosis Inducing Agents

5× solutions of apoptosis inducing agents were prepared: staurosporine at 25 μM and Fludarabine at 5 μM. ZVAD was prepared at 5× as well at a concentration of 500 μM. Combination preparations of staurosporine or fludarabine plus ZVAD were also prepared at 5×.

Solutions of apoptosis inducing agents, ZVAD, and media controls were arrayed into a 96-well deep well plate. The corresponding daughter plates for apoptosis conditions were removed from the 37° C. incubator. Facilitating the use of the Liquidator, 140 μL of 5× drug was aspirated from the 96-well deep well plate and added to the cells in the apoptosis daughter plate. The daughter plate was pulse-vortexed for 5 seconds and placed into a 37° C., 5% CO₂ incubator for 48 hours.

After 48 hours the cells were washed twice with PBS and centrifuged at 4000×g for 8 minutes at room temperature. Plates were pulse-vortexed to disrupt cell pellet and 2004, of 1× Amine Aqua (Invitrogen) was added to the cells. Plates were placed in 37° C., 5% CO₂ incubator for 15 minutes.

After 15 minute incubation 1 mL RPMI1640 1% FCS was added to each well and plates were centrifuged at 1200 RPM for 8 minutes at room temperature. Supernatant was aspirated and 200 μL of RPMI1640 1% FCS was added to each well followed by 2004 of 3.2% PFA to fix the cells. Plates were incubated in at 37° C. water bath for ten minutes then centrifuged at 1000×g for 8 minutes at room temperature. Plates were pulse-vortexed to disrupt cell pellets and 600 μL of ice-cold methanol was added to each well to permeabilize the cells. Plates were sealed with adhesive foil covers and placed at −80° C. overnight.

Intracellular Staining of Cells

100 uL of sample suspension from apoptosis plates were aliquoted into 3 separate deep-well 96-well plates. 1 mL of FACS Buffer (PBS 0.5% BSA, 0.05% NaN₃) was added to each well and plates were centrifuged at 1000×g for 8 minutes at room temperature. The supernatant was aspirated and samples were washed again with 1 mL FACS Buffer and centrifuged at 1000×g for 8 minutes at room temperature.

Antibody cocktails for signaling and apoptosis readouts were prepared in FACS Buffer. All cocktails contained a common panel of fluorochrome conjugated mAbs against cell surface antigens: CD3—Pacific Blue, CD20—PerCPCy5.5, CD5—biotin. Antibodies for each of the signaling panels are as follows: panel 1: pAkt—Alexa Fluor 488, pSyk—(Phycoerythrin) PE, pBLNK—Alexa Fluor 647; panel 2: pS6—Alexa Fluor 488, pPLCγ2—PE, pLyn—Alexa Fluor 647; panel 3: pErk—Alexa Fluor 488, SHP-1 purified; panel 4: SHP-2 purified, pSTAT5—PE, p-65/RelA—Alexa Fluor 647. Antibody cocktails were aliquoted into corresponding deep-well 96-well plates for staining

TABLE 1
Antibody panels used for measurements of signaling downstream
of BCR, apoptosis and delineation of cell subsets.

| | Ax488 | PE | Ax647 |
|---|---|---|---|
| Signaling Panel 1 | p-Akt(S473)* | p-Syk(Y352)/ | p-BLNK(Y84) |
| | | p-ZAP-70(Y319) | |
| Signaling Panel 2 | p-S6(S235/S236)* | p-PLCg2(Y759) | p-Lyn(Y505) |
| Signaling Panel 3 | p-Erk(T202/Y204) | Empty | SHP-1** (2° Goat-anti- |
| | | | rabbit-Ax647) |
| Signaling Panel 4 | SHP-2* (2° Goat-anti- | p-STAT5(Y694) | p65/RelA(S529) |
| | rabbitAx488) | | |
| | FITC | PE | Ax647 |
| Apoptosis Panel 1 | Cleaved Caspase 3 | Cleaved PARP | Cytochrome C |
| Apoptosis Panel 2 | Empty | Cleaved PARP | p-Chk2(T68)* (2° GaR- |
| | | | Ax647) |
| | FITC | PE | APC |
| Phenotypic Panel 1 | IgM | IgD | IgG |
| Phenotypic Panel 2 | λ-light chain | κ-light chain | CD38 |
| Phenotypic Panel 3 | CD45 | CD79β | CD22 |

All antibodies are from Becton Dickinson Biosciences (San Jose, CA) unless otherwise noted.

500 μL of cell suspensions in methanol from the signaling plates were aliquoted into a separate deep-well 96-well plate. Plates were centrifuged at 1000×g for 8 minutes at room temperature.

Supernatant was aspirated and 1 mL of FACS Buffer (PBS 0.5% BSA, 0.05% NaN₃) was added to wash the cells. Plates were centrifuged at 1000×g for 8 minutes, supernatant aspirated and 1 mL wash repeated. After the second wash, samples were resuspended in 400 μL of FACS Buffer. 100 μL from each signaling plate was delivered to each of the staining plates with pre-aliquoted antibody cocktail. Plates were sealed with adhesive foil, pulse-vortexed for 7 seconds, and placed in 4° C. shielded from light for 16 h.

Antibody cocktails for apoptosis readouts were prepared in FACS Buffer. All cocktails contained a common panel of fluorochrome conjugated mAbs against cell surface antigens: CD3Pacific Blue, CD20—PerCPCy5.5, CD5—biotin. Antibodies for each of the apoptosis panels are as follows: panel 1: Cleaved Caspase 3—FITC, Cleaved PARP—PE, Cytochrome C—Alexa Fluour 647; panel 2: BCL-2—FITC, Cleaved PARP—PE, Cleaved Caspase 8 purified, panel 3: Cleaved PARP—PE, pChk2 purified. Antibody cocktails were aliquoted into corresponding deep-well 96-well plates for staining.

500 μL of cell suspensions in methanol from the apoptosis plates were aliquoted into a separate deep-well 96-well plates. Plates were centrifuged at 1000×g for 8 minutes at room temperature. Supernatant was aspirated and 1 mL of FACS Buffer (PBS 0.5% BSA, 0.05% NaN₃) was added to wash the cells. Plates were centrifuged at 1000×g for 8 minutes, supernatant aspirated and 1 mL wash repeated. After the second wash samples were resuspended in 300 μL of FACS Buffer. 100 μL from each signaling plate was delivered to each of the staining plates with pre-aliquoted antibody cocktail. Plates were sealed with adhesive foil, pulse-vortexed for 7 seconds, and placed in 4° C. shielded from light for 16 h.

After 16 h incubation both the signaling and apoptosis plates were processed similarly. 1 mL of PBS was added to each well and plates were centrifuged at 1000×g for 8 minutes at room temperature. Supernatant was aspirated and 104, of 2° staining cocktail was added to the residual volume in the wells. Every well received streptavidin Qdot605 (0.25 μl in 104). For wells stained with SHP2 2° goat anti-rabbit Alexa Fluor 488 (1:5000) was added in addition to streptavidin Qdot605. For wells stained with SHPT or pChk2, goat anti-rabbit Alexa Fluor 647 (1:5000) was added in addition to streptavidin Qdot605 (the combination comprises the secondary stain). Samples were incubated with secondary stain was incubated at room temperature for 30 mins shielded from light.

After incubation with secondary stain, plates were washed with 1 mL of FACS Buffer and centrifuged at 1000×g for 8 minutes at room temperature. Supernatant was aspirated and plates were pulse-vortexed for 7 seconds to disrupt the pellet. 804 of FACS Buffer was added to the residual volume in the wells. Using the Liquidator 96-well pipettor, samples were transferred from deep-well 96-well plates to standard profile 96-well u-bottom plates.

Samples were acquired on a BD FACSCantoII flow cytometer.

Surface marker studied were CD3−/CD20+/CD5+ (gating); IgM/IgD/IgG/λLC/κLC/CD79b/CDE19 (BCR); and CD38/CD22/CD45. Modulators used were anti-μ with or without H₂O₂, or anti-γ with or without H₂O₂, and PMA. Signaling and phosphatase expression molecules analyzed included four panels: 1) p-Akt/p-Syk/p-BLNK; 2) p-56/p-PLC γ2/p-Lyn; 3) p-Erk/SHP-1; and 4) SHP-2/p-STAT5/p-p65.

Evaluation of DNA damage and apoptosis was made after exposure of samples to the following modulators: F-ara-A (Fludarabine) alone, staurosporine alone, ZVAD alone or the combination of F-ara-A with ZVAD and staurosporine and ZVAD and involved 3 panels of antibodies that recognized activated proteins within these pathways: 1) c-Caspase 3/c-PARP/cyt C; 2) Bcl-2/c-Caspase 8/c-PARP; and 3) p-Chk2/c-PARP.

Each determination was made in duplicate and correlation coefficients (r2 values) were greater than 0.8 in most cases.

Cells are deemed sensitive or responsive to F-ara-A as measured by apoptosis markers cleaved caspase and PARP.

Evaluating the physical consequences of apoptosis on the cells (namely plasma membrane blebbing and breakdown) showed a modest increase in cell death by amine aqua in F-ara-A treated or untreated cells.

Of the 110 nodes measured per sample, 106 had R²>0.8. The four nodes where R2 was less could be accounted for by outliers.

Basal levels of phosphorylation are more variable in CLL samples than in healthy B cells

### Results

Surface marker characterization and basal phosphorylation states in the CLL cohort

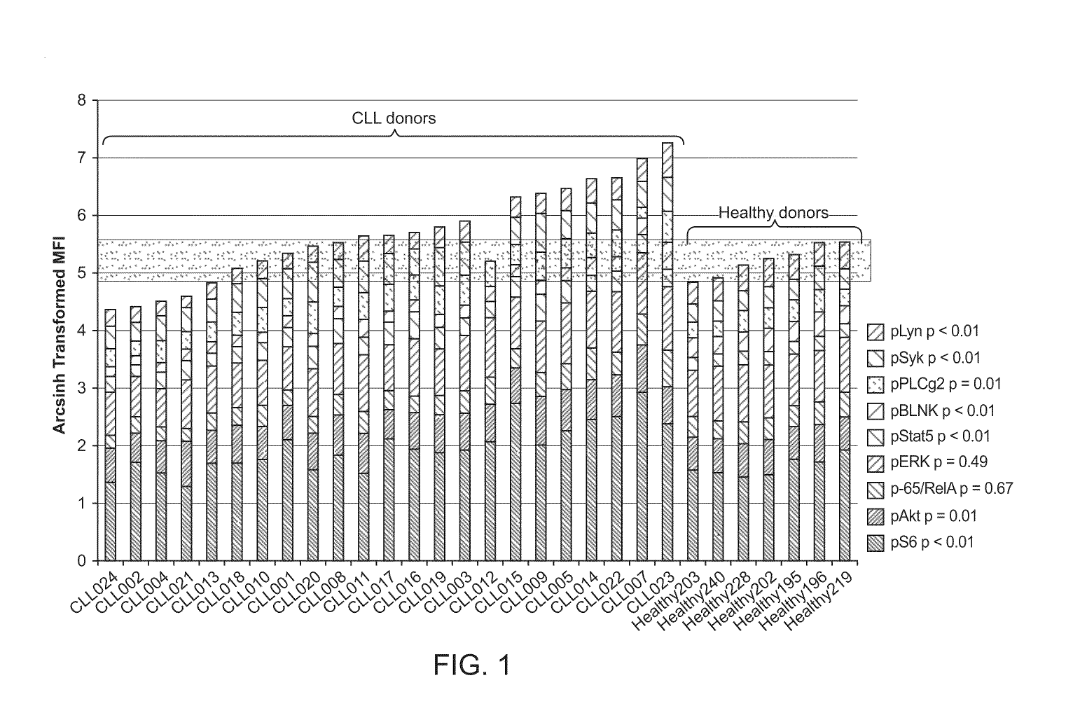

Comparison of MFI values of BCR signaling molecules in their basal phosphorylation states showed greater variability in CLL versus healthy B cells (FIG. 1). MFI values for p-Akt and p-Lyn spanned a range of 16 and 17 respectively among healthy B cells and 63 and 66 in CLL B cells. p-Erk and p-65/RelA showed no significant differences between healthy and CLL samples, indicating that at their basal level the activation state of these molecules did not reflect a CLL-dependent phenotype.

Expression of markers (determined as MFIs) associated with the B cell lineage, and tyrosine phosphatases (CD45, SHP-1 and SHP-2), were compared between healthy and CLL B cells. Surface marker expression was homogeneous in B cells from healthy donors whereas in CLL B-cells surface marker expression was more heterogeneous. The expected 2:1 kappa/lambda ratio was evident in healthy B cells, and contrasted with the distorted ratios observed in CLL samples indicating clonal expansion of a malignant cell. (See Hamblin T J. Best Pract Res Clin Haematol. 2007; 20:455-468). A subset (13/23) of CLL samples expressed kappa chain exclusively, and a further subset of 7/23 CLL samples expressed only lambda chain. In 3 samples no light chain was detected suggesting clonal expansion of an immature B cell. Expected hallmarks of CLL were seen in the low expression of IgM and CD79β in individual patient samples. No statistical classification of CLL samples into distinct subgroups could be made based on expression levels of the measured markers and tyrosine phosphatases.

### Modulated Signaling Responses Distinguish Subgroups of CLL Patient Samples

To test whether phenotypic characterization of CLL physiology could be discerned based on responses of cells to extracellular stimuli, modulated BCR intracellular signaling was determined either in response to anti-g (ligand-dependent) or post H₂O₂ treatment to evaluate the contribution of tonic signaling (ligand-independent) to BCR output. (See Irish J M, J. Immunol. 2006; 177:1581-1589; Monroe J G. Nat Rev Immunol. 2006; 6:283-294; and Wienands J Proc Natl Acad Sci USA. 1996; 93:7865-7870). Samples were treated with anti-g alone, H₂O₂ alone (3.3 mM) or the combination for 10 minutes to recognize differences in BCR signaling between CLL and healthy B cells. The 10-minute time point was chosen based on kinetic analyses in order to see robust, but not necessarily maximal phosphorylation, of all the BCR pathway signaling molecules under study. H₂O₂ titrations were performed and the concentration chosen was one in which minimal effects were seen on canonical signaling in healthy B cells. The millimolar concentration requirement for H₂O₂ is consistent with its intracellular millisecond half-life (See Reth M. Nat. Immunol. 2002; 3:1129-1134).

TABLE 2
BCR and apoptosis responses in CLL and healthy samples.

| Sample | CLL | | | | | | | | |
|---|---|---|---|---|---|---|---|---|---|
| Group | Sample | p-Lyn | p-Syk | p-BLNK | p-STAT5 | p-PLC-γ | p-Akt | p-Erk | Apoptosis |
| Group 1 | CLL014 | 43 | 52 | 52 | 36 | 67 | 59 | 57 | ✓ |
| Group 1 | CLL003 | 73 | 74 | 64 | 60 | 81 | 74 | 86 | ✓ |
| Group 1 | CLL016 | 27 | 26 | 17 | 23 | 39 | 56 | 41 | ✓ |
| Group 1 | CLL008 | 57 | 57 | 47 | 36 | 77 | 67 | 84 | ✓ |
| Group 1 | CLL024 | 80 | 65 | 73 | 47 | 77 | 46 | 88 | ✓ |
| Group 1 | CLL013 | 19 | 25 | 19 | 13 | 37 | 26 | 27 | ✓ |
| Group 1 | CLL010 | 56 | 54 | 39 | 30 | 85 | 80 | 90 | ✓ |
| Group 1 | CLL002 | 39 | 39 | 36 | 21 | 56 | 40 | 70 | ✓ |
| Group 1 | CLL001 | 40 | 41 | 20 | 34 | 51 | 43 | 52 | ✓ |
| Group 1 | CLL004 | 20 | 23 | 19 | 13 | 45 | 31 | 76 | ✓ |
| Group 1 | CLL018 | 20 | 21 | 18 | 15 | 33 | 45 | 61 | ✓ |
| Group 1 | Mean | 43 | 43 | 37 | 30 | 59 | 52 | 67 | |
| Group 1 | SD | 21 | 18 | 20 | 15 | 19 | 17 | 21 | |
| Group 2 | CLL009 | 47 | 63 | 54 | 29 | 79 | 56 | 69 | — |
| Group 2 | CLL019 | 24 | 29 | 19 | 12 | 40 | 66 | 53 | — |
| Group 2 | CLL020 | 10 | 5 | 5 | 7 | 33 | 40 | 44 | — |
| Group 2 | CLL023 | 10 | 16 | 14 | 10 | 43 | 87 | 83 | — |
| Group 2 | CLL011 | 5 | 7 | 5 | 3 | 28 | 68 | 19 | — |
| Group 2 | CLL012 | 17 | 32 | 23 | 7 | 48 | 61 | 34 | — |
| Group 2 | CLL021 | 6 | 8 | 9 | 6 | 7 | 8 | 10 | — |
| Group 2 | CLL005 | 15 | 18 | 10 | 9 | 35 | 74 | 41 | — |
| Group 2 | CLL007 | 8 | 7 | 0 | 4 | 19 | 66 | 40 | — |
| Group 2 | Mean | 12 | 15 | 11 | 7 | 32 | 59 | 41 | |
| Group 2 | SD | 6 | 10 | 7 | 3 | 13 | 23 | 21 | |
| Healthy | CON219 | 1 | 3.5 | 3 | 0.6 | 25 | 44 | 26 | ✓ |
| Healthy | CON196 | 3 | 5 | 6 | 0.16 | 12 | 48 | 15 | ✓ |
| Healthy | CON195 | 4 | 6 | 10 | 3.4 | 18 | 51 | 21 | ✓ |
| Healthy | CON240 | 1 | 1 | 3 | 0.6 | 4.5 | 38 | 10 | ✓ |
| Healthy | CON202 | 0.6 | 3 | 5 | 0.6 | 5 | 45 | 17 | ✓ |
| Healthy | CON228 | 1 | 4 | 5 | 1 | 4 | 30.5 | 29 | ✓ |
| Healthy | CON 203 | 0.5 | 1.2 | 2 | 4 | 0.3 | 33 | 6 | |
| Healthy | Mean | 2 | 3 | 5 | 1 | 10 | 41 | 17 | |
| Healthy | SD | 1 | 1 | 2 | 1 | 7 | 7 | 7 | |
| | Ramos | 50 | 53 | 88 | 38 | 77 | 85 | 64 | ✓ |

Numbers are percentages of CLL B cells within a sample that undergo peroxide mediated phoshphorylation of intracellular signaling molecules.

Consistent with previous reports, anti-g-mediated BCR signaling was further potentiated by H₂O₂ in B cells from healthy donors. See Irish, J. Immunol., 2006.

Analysis of the signaling responses showed that the CLL sample cohort could be broadly segregated into two patient groups. In Group 1 a significant subpopulation of cells was responsive to H₂O₂ (for example the mean percentage of a cell subset with a responsive Lyn, Syk or BLNK population was 43%, 43% and 37% respectively, Table 2 and FIG. 2(A)). In all but three cases, the addition of anti-μ did not mediate a further increase in downstream signaling responses, consistent with the notion that aberrant phosphatase activity might be regulating BCR activity in CLL signaling. (The numbers in Table 2 are derived from 2D contour plots, such as those shown in FIGS. 2 A, B, and C. Gates are manually set and then applied to all samples. The numbers in Table 2 can vary based on slightly different gate placements.)

In Group II there was a reduced number of such cells after exposure to H₂O₂. For example, the mean percentage of cells with activated Lyn, Syk or BLNK was 12%, 15% and 11% respectively (Table 2 and FIG. 2(B)). On the lower side, CLL021 showed 5-6% and CLL007 showed approximately 2% B cells with activated Syk and BLNK.

Signaling in patient CLL samples was coordinated in that all the measured components of the canonical B cell receptor network, (p-Lyn, p-Syk, p-BLNK, p-PLCγ2) were augmented, in concert. In Group II, although the H₂O₂-mediated signaling response of the proximal BCR effectors was severely abrogated the p-Akt response was similar between the two groups (52% for Group 1 and 59% for Group lII, Table 2). The activation of Erk in Group II was less than in Group I (41% and 67% respectively). In healthy B cells, all signaling molecules except Akt were minimally responsive to H₂O₂ treatment alone (Table 2). Given that H₂O₂ is a known inhibitor of phosphatase activity, and that phosphatase activation is a physiological regulator of proximal BCR signaling activities, (J Irish Blood, 2006, Reth Nat. Immunol. 2002, Singh D K, Cell. 2005; 121:281-293, J Irish J. Immunol, 2002, Monroe J G. Nat Rev Immunol. 2006; 6:283-294, Wienands J,. Proc Natl Acad Sci USA. 1996; 93:7865-7870 and Rolli V, Mol Cell. 2002; 10:1057-1069) these data suggest that deregulation of phosphatase activity could explain some of the differences observed between CLL and healthy B cell signaling responses.

Unexpectedly, in 14/23 CLL samples there was an increase in phosphorylated STAT5 in response to H₂O₂ within a subset of cells in individual samples (FIG. 2(C)(left hand panels). In 7/23 CLL samples as well as in healthy B cells a minimal number of cells exhibited an increase in phosphorylated STAT5 in response to H₂O₂ (FIG. 2(C)(right hand panels). This observation suggests either that there is a significant re-wiring event downstream of tonic BCR signaling or that an alternative pathway is activated, and either could be connected to STAT5 activity.

Samples that showed an H₂O₂-mediated p-STAT5 response were the same as those in which the canonical components of the BCR network were activated in response to H₂O₂ (Table 2). Interestingly, in many patient samples at least two prominent CLL cell populations with unique and definable signaling responses were observed. For example, a sample in which a dominant cell subset demonstrated augmented signaling in response to H₂O₂, other subsets could be identified with marginal responses (FIG. 2(B-C)). No such distinctions were observed using basal phosphorylation states, underscoring that activation of BCR signaling molecules highlights the differences in pathway biology between and within samples.

Lack of responsiveness of the Lyn/Syk/BLNK/PLCγ2 signaling proteins to H₂O₂ treatment was associated with lack of apoptotic response in CLL B cells.

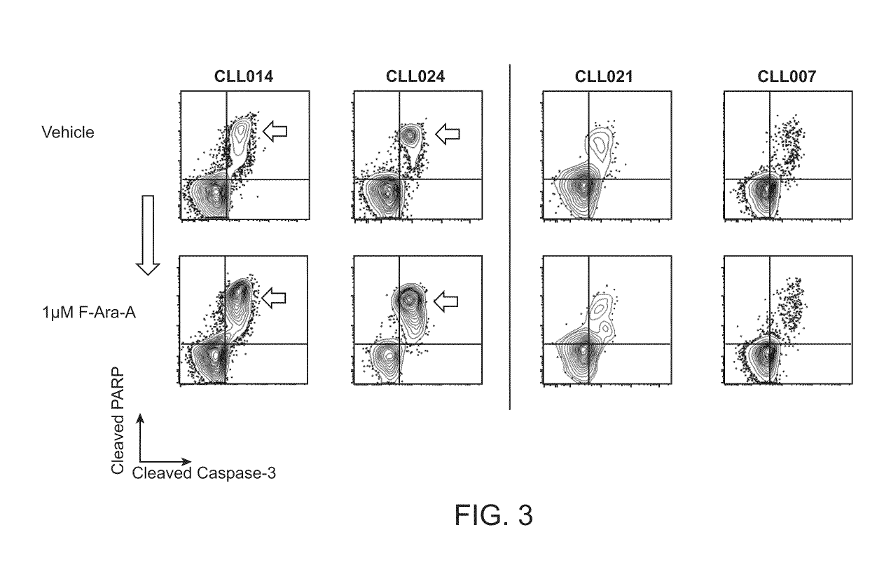

There has long been a presumed link between ligand-induced BCR signaling, tonic BCR signaling, and B cell survival. (See Kraus M, et al Cell 2004; 117:787-800 and Kraus M J Exp Med. 2001; 194:455-469). If such links are critical, then it might be further postulated that in CLL and other B cell malignancies, associations might be present between the observed signaling potential downstream of the BCR. To test this, apoptotic responses of CLL samples and healthy donors were enumerated by SCNP after in vitro exposure to F-ara-A for 48 hours. Representative CLL samples that were responsive or refractory to in vitro F-ara-A exposure are depicted by correlated measurement of cleaved caspase 3 and cleaved PARP in each cell (FIG. 3 Measurements of loss of mitochondrial cytochrome C in the same cells are consistent with the apoptotic responses.

Within responsive samples there were at least two cell subpopulations, with a second cell subset that was refractory to in vitro F-ara-A exposure (FIG. 3 (Left hand panels). This is reminiscent of the signaling data described above in which cell subsets with heterogeneous signal transduction responses were seen within the same sample (FIG. 2(A)). DNA damage was assessed using antibodies against the phospho-threonine 68 epitope on Chk2, the ATM phosphorylation site (F-ara-A incorporation into DNA results in a damaged product and activation of cell cycle checkpoint kinases). (See Antoni L, Nat Rev Cancer. 2007; 7:925-936). Although differences in p-Chk2 levels were seen in cell subsets within F-ara-A responsive and refractory samples, these differences were not statistically significant.

Given the pro-survival role BCR signaling plays in healthy and tumorigenic B cell biology (See Irish Blood 2006, Brerski R J, Monroe J G. Bioscience; 2008; Irish J. Immunol 2006; and Jumaa H, Annu Rev Immunol. 2005; 23:415-445) the data were analyzed for any associations between H₂O₂-modulated BCR signaling and apoptotic response to in vitro F-ara-A exposure. To evaluate the CLL cohort for trends, all cell events from the gated B cells of all CLL samples and, separately, all healthy samples were combined into respective ‘virtual’ samples that represented a composite of signaling for each modulated signaling molecule (FIG. 4(A) healthy B cells (pink-narrow peak) and CLL B cells (cyan-broad peak)). On the assumption that at least two subpopulations of cells could be driving the distribution of expression in the combined samples, the underlying “subpopulations” were decomposed via mixture modeling for the CLL samples to represent the underlying probability distributions (FIG. 4(B)).

Identifying Cell Populations with Distinct Signaling by Mixture Models

SCNP measures signaling for each cell individually, allowing characterization of a spectrum of cell signaling responses. Cells were gated on light scatter characteristics and then evaluated for viability by exclusion of Amine Aqua. Live cells were gated as CD3−/CD20+ and then evaluated for CD5 expression. Metrics including median fluorescent intensity (MFI), percentage of positive cells, and mixture-model derived population content (see below), were extracted from CD3−/CD20+ cells. FCS files were analyzed in FlowJo (Treestar, Ashland, Or) version 8.8.2 Plotting a histogram of the distribution of fluorescence intensities of all cells across all samples suggests that there are often distinct populations of cells with different signaling characteristics (FIG. 1). Expectation maximization methods (Hastie, Tibshirani, and Friedman, The Elements of Statistical Learning, pp 236-243, 2001). were applied to histograms of arcsinh transformed fluorescence intensities to generate mixture models (See Efroni S, Schaefer C F, Buetow K H (2007) Identification of Key Processes Underlying Cancer Phenotypes Using Biologic Pathway Analysis. PLoS ONE 2(5): e425. doi:10.1371/journal.pone.000042) comprised of two normal distributions using the mixdist package (see Peter Macdonald and with contributions from Juan Du (2008). mixdist: Finite Mixture Distribution Models. R package version 0.5-2. http://www.r-project.org, http://www.math.mcmaster.ca/peter/mix/mix.html) for R (see R Development Core Team (2009). R: A language and environment for statistical computing. R Foundation for Statistical Computing, Vienna, Austria. ISBN 3-900051-07-0, URL http://www.R-project.org). The population distributions observed in the H₂0₂-treated samples are evidence of heterogeneity in the phosphatase activity that regulates tonic BCR signaling among different CLL samples. Metrics were defined to characterize each patient sample as to the extent to which it contains cells in each population by computing the area under the curve for the fluorescent intensities from that sample with respect to a random sampling of 50000 events representing each mixture-model derived distribution. These metrics were termed ‘MixMod1’ and ‘MixMod2’ representing the areas under the curve for the distributions with lower and higher mean fluorescent intensities, respectively.

Striking differences were observed in the population distributions between healthy versus CLL B cell populations after treatment with H₂O₂ alone (see arrows in FIG. 4(A)). The histograms show a greater spread in the fluorescence intensities in CLL versus healthy B cells for the measured BCR signaling molecules (FIG. 4(A) CLL B cells (cyan-broad peak) versus (healthy B cells (pink-narrow peak)). Combining H₂O₂ with anti-g did not produce additional substantial changes in the B cell population distribution of CLL B cells, suggesting that H₂O₂-mediated phosphatase inhibition was defining the signaling potential of these CLL B cell populations. This is surprising and contrasted with healthy B cells in which the combination of H₂O₂ and anti-g resulted in an enhanced population distribution based on signaling, compared to each of these modulators alone (FIG. 4(A), fourth column, see Irish Blood 2006). A comparison of the histograms for healthy B cells versus CLL B cells in the absence of a modulator show that there were only minor differences in basal levels of phosphorylation for each BCR signaling molecule (this differs from the bar chart in FIG. 1 in which differences in basal phosphorylation between CLL and healthy B cells were computed on a per patient basis).

The trends in the mixture models emphasize the patterns (as expected) of the individual patient samples: the presence of an H2O2 de-repressed cell subpopulation and quiescent cell subset. The mixture model has the benefit of showing, at least for this cohort of patients, the averaged boundaries of where such subpopulations of cells lay on the histograms. The metrics that defined these curves were next used to develop classifiers (see below) for responses that might be linked to the presence of absence of these observed cell subsets.

Receiver operating characteristic (ROC) curves were generated to determine whether presence of either or both of the two populations defined by the mixture models was associated with response or lack of response to in vitro exposure to F-ara-A (FIG. 5(A). No such associations could be determined for healthy B cells, as expected, since the H₂O₂ concentration was selected to give no response in healthy B cells as previously reported. (See Irish J Immunol 2006).

Area under the ROC curves (AUC of ROC curve) (See Hanley J A, Radiology. 1982; 143:29-36) for signaling induced by H₂O₂ treatment showed that p-Lyn (AUC 0.84), p-Syk (AUC 0.75), p-BLNK (AUC 0.79), p-PLCγ2 (AUC 0.81), p-Erk (AUC 0.77) and p-STAT5(AUC 0.84) signaling stratifies patient samples according to their apoptotic pathway response (FIG. 5(A). Using the metrics derived from these mixture models, the AUC plots demonstrated that samples in which signaling was revealed by H₂O₂ exposure were more likely to undergo F-ara-A mediated apoptosis (FIG. 4(B), 5(A)). By contrast, samples in which H₂O₂ failed to induce signaling were largely non-responsive to F-ara-A (FIGS. 2(A-C), FIG. 3 and Table 2). An un-scaled mixture model of H₂O₂-induced phosphorylation of STAT5, (see FIG. 4(B), row 5 from the top, 3rd column), (AUC 0.84 from FIG. 5(A)) was established for the cohort of CLL samples (FIG. 5(B), top panels). Of note, the range of expression observed for SHP-1, SHP-2 and CD45 tyrosine phosphatases was greater in CLL compared to healthy B cells. However, there was no association with the expression levels of these markers with either the magnitude of induced signaling or apoptotic responsiveness. Thus, levels of these phosphatases alone were not surrogates for these pathway functions.

The ROC curves (FIG. 5(A)) demonstrated significant associations between H₂O₂-mediated signaling and apoptotic proficiency. The samples could be divided into two predominant response phenotypes. First, samples CLL007 and CLL021 are exemplary of patients that showed a single major H₂O₂ non-responsive population of cells (FIGS. 5B and 2B). As noted, these patient samples were refractory to F-ara-A exposure in vitro and had a reduced H₂O₂-mediated activation of Lyn, Syk, BLNK, PLCγ, or STAT5. A second phenotypic response group, represented by samples CLL014, and CLL024 were responsive to F-ara-A and had significant activation of Lyn, Syk, BLNK, PLCγ, and STAT5 and whose expression profiles overlapped areas defined by the individual distributions of the mixture model, and in some cases (CLL014 being representative) demonstrate a clear bimodal phenotype (FIG. 5 B, 2A). There were two outliers for which this association did not hold. CLL009 exhibited a robust H₂O₂-mediatated signaling response for all measured signaling molecules and yet failed to undergo apoptosis (FIG. 5(B) Table 2). These data suggest that in these samples a different biology may be driving CLL, indicating that despite the broad associations observed in signaling responses including STAT5 downstream of the BCR to the apoptotic response, there remain additional linkages between these signaling systems that can vary independently. SCNP Improves In Vitro Fludarabine Response Prediction in CLL Cells that are ZAP-70 positive and IgV_{H} Unmutated Cells when Analyzed Separately from all CLL Cells

No associations could be made between the IgV_{H} mutational status or ZAP-70 expression status and in vitro response to F-ara-A (AUC value for ZAP-70 and apoptotic response 0.53 and Fisher's exact test for association between IgV_{H} status and apoptosis (F-ara-A responder/F-ara-A refractory, p value=1, odds ratio=0.1.2). See Table 3 below.

TABLE 3
ZAP-70 and IgV_{H} mutational status do not discriminate
in vitro fludarabine responders from non-responders

| | Fludarabine responders | Fludarabine non-responders |
|---|---|---|
| ZAP+ | 4 | 4 |
| ZAP− | 5 | 6 |

Fisher exact test: p = 1, odds-ratio = 1.2, data is same for IgV_{H} mutational status

However, SCNP improved in vitro fludarabine response prediction when applied to CLL patient cells that were ZAP-70 positive or IgV_{H} unmutated. ZAP-70 and IgV_{H} mutational status are used to classify patients to inform clinical decisions. Splitting patients according to their ZAP-70 status, as defined by ZAP-70 measured using flow cytometry being>20% (that is, ZAP-70>20% is ZAP positive), or IgV_{H} mutational status improves in vitro fludarabine response prediction using SCNP in the ZAP-70 positive or IgV_{H} unmutated group, as measured by increase in AUC values in an ROC curve generated from fold change analysis. Compare the AUC values for p-Lyn stimulated by H₂O₂ (0.81 split/0.79 unsplit), p-Syk (0.88 split/0.76 unsplit), p-BLNK (0.88 split/0.81 unsplit), p-PLCg2 (0.88 split/0.76 unsplit), and p-STAT5(1.0 split/0.88 unsplit). See Table 4 below.

TABLE 4
SCNP improves in vitro fludarabine response prediction in ZAP+
and IgVH unmutated CLL patient cells measured by H202 modulation

| | AUC for in vitro | AUC for in vitro |
|---|---|---|
| | fludarabine response | fludarabine response |
| | in ZAP+/IgVH | in entire (unsplit) |
| Node | unmutated patients | patient group |
| H₂O₂/p-Lyn | 0.81 | 0.79 |
| H₂O₂/p-Syk | 0.88 | 0.76 |
| H₂O₂/p-BLNK | 0.88 | 0.81 |
| H₂O₂/p-PLCg2 | 0.88 | 0.76 |
| H₂O₂/p-STAT5 | 1.0 | 0.88 |

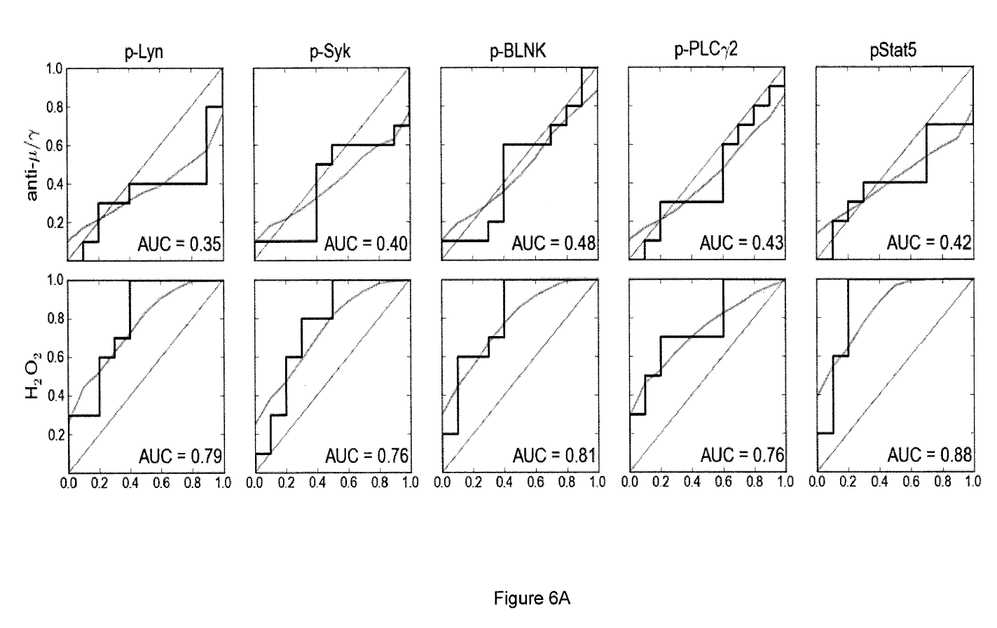

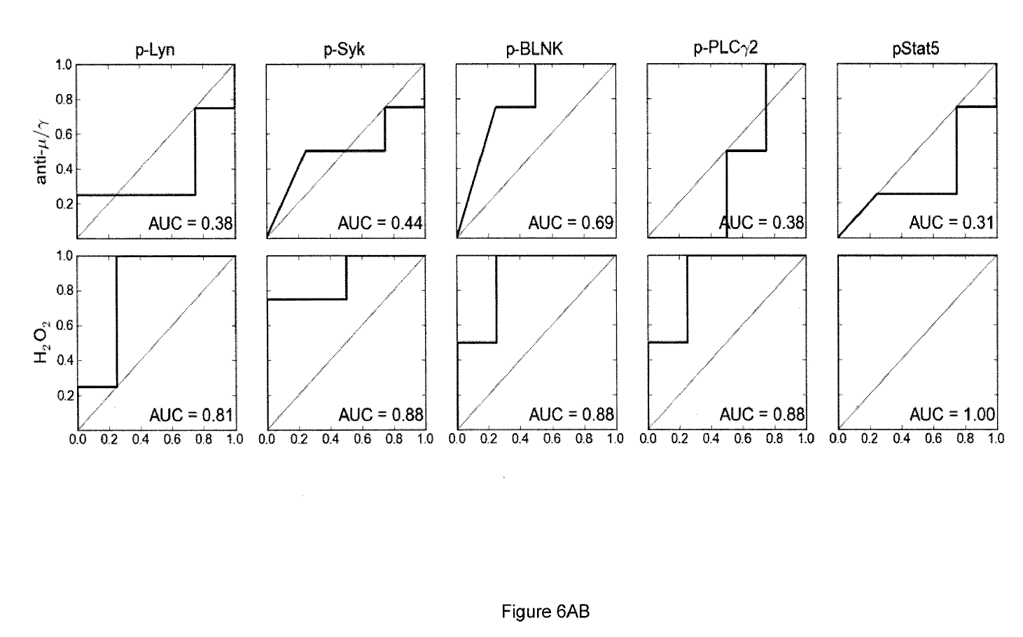

FIG. 6 shows statistical association between H₂O₂-mediated signaling and apoptosis induction by F-ara-A (Fludarabine) in the group comprised of all CLL cells regardless of ZAP-70 or IgV_{H} mutational status compared with the group comprised of ZAP-70 positive or IgV_{H} unmutated status. (A) ROC curves from a fold change model were expressed in order to evaluate how statistically significant H₂O₂-induced signaling is in predicting an in vitro apoptotic response to F-ara-A for all CLL cells, regardless of ZAP-70 or IgV_{H} mutational status (that is, prediction of apoptotic response is based on H₂O₂-induced nodes). The fold change metric for H₂O₂-mediated signaling was used to calculate whether there was an association with response or lack of response to in vitro exposure to F-ara-A. A value of 0.5 for the ROC plots indicates that the association is due to chance. A value of 1.0 indicates that there is a perfect association. (B) ROC curves from a fold change model were expressed with 95% confidence limits to evaluate how statistically significant H₂O₂-induced signaling is in predicting in vitro apoptotic response to F-ara-A for cells with ZAP-70 positive or IgV_{H} unmutated status (that is, prediction of apoptotic response is based on H₂O₂-induced nodes in combination with ZAP-70 or IgVH status).

### Discussion

Recently, several molecular and cytogenetic lesions have emerged as potential prognostic indicators for CLL. However, there are many disparities and confounding issues limiting their clinical utility. (See Hallek M, Guidelines for the diagnosis and treatment of chronic lymphocytic leukemia: a report from the International Workshop on Chronic Lymphocytic Leukemia updating the National Cancer Institute-Working Group 1996 guidelines. Blood. 2008; 111:5446-5456; Hamblin T J. Best Pract Res Clin Haematol. 2007; 20:455-468; Hamblin T J, Blood. 1999; 94:1848-1854; and Kay N E, Leukemia. 2007; 21:1885-1891). For example, although primary resistance to fludarabine has been shown to occur in patients harboring p53 deletions, a recent study reported that treatment-naïve patients with p53 deletions exhibit clinical heterogeneity with some patients experiencing an indolent course. (See Tam CS, Blood. 2009; 114:957-964 and Dohner H, Blood. 1995; 85:1580-1589). These published clinical studies suggest that there are underlying differences in CLL biology, which if understood, could provide more reliable prognostic information in individual patients.

The data in this study have highlighted a link between H₂O₂-induced changes in phosphorylation of BCR signaling proteins and F-ara-A-mediated apoptosis in CLL B cells. H₂O₂, a second messenger acts by oxidizing cysteines with pKa values below 5.0, such as are found in protein tyrosine phosphatases to sulfenic acid (See Reth, 2002). As an oxidant, H₂O₂ has other activities, these data potentially support a mechanism whereby deregulation of the kinase/phosphatase equilibrium results in activation of signaling proteins within the BCR network. Regardless of its exact mechanism of action, H₂O₂ was able to reveal differential signaling within CLL samples and these signaling differences appear to be associated with a signaling posture that either drives, or is driven by the ability of these cells to respond to apoptotic induction, in this case F-ara-A.

By analyzing signaling on a cell by cell basis, single cell network profiling (SCNP) allowed characterization of a spectrum of cell signaling responses. Single cell analysis, combined with mixture modeling identified at least two phenotypes for CLL B cells in human patients based on their response or lack of response (for proximal BCR signaling molecules) to H₂O₂ (FIG. 3(A), (B)). In samples where signaling is revealed by H₂O₂ a deregulated phosphatase near the BCR and/or other tyrosine kinase receptor signaling system(s) could be dampening signaling of BCR signaling molecules. Notably, some patients demonstrated simultaneous presence of both cell subsets, suggesting co-evolution of signaling phenotypes, a common precursor of these cell subsets, or a lineage relationship between the two subpopulations of cells (FIG. 2(A), (B), (C)). For most of the samples in which H₂O₂-mediated signaling was observed there was an association with an apoptotic response to in vitro F-ara-A exposure with AUCs of 0.8 as strong predictors for F-ara-A-induced apoptosis using H₂O₂-mediated increases in p-Lyn, p-PLCγ-2 and p-STAT5 as a surrogate (FIG. 5(A). Interestingly, and in contrast to studies where the presence of ZAP-70 and unmutated IgV_{H} correlated with greater anti-g-mediated-BCR signaling (See Chen L, Blood. 2008; 111:2685-2692 and Efremov DG, Autoimmun Rev. 2007; 7:102-108), the signaling responses described here were unrelated to the IgV_{H} mutational status or to ZAP-70 expression and spanned a range of cytogenetic abnormalities. It is important to note that the above studies (Chen Blood 2008, Gobessi S, Leukemia. 2009; 23:686-697, and Efremov Autoimmun Rev.2007) were accomplished via indirect assay of total phosphotyrosine on signaling proteins in each report. In our study, we undertook direct assay of phosphorylation sites using antibodies directed against known, functional, epitopes.

No associations were observed between either CD22 or CD45 expression levels with H₂O₂— mediated signaling. The contribution of phosphatases to tonic BCR signaling is further substantiated by the global inactivation of tyrosine phosphatases by sodium pervanadate or H₂O₂. These agents conferred de novo phosphorylation of BCR effector molecules that would normally be phosphorylated by ligand-dependent BCR aggregation. (See Reth M. Nat. Immunol. 2002; 3:1129-1134 and Wienands J, Proc Natl Acad Sci USA. 1996; 93:7865-7870). An independent study showed that in CLL B cells where Lyn protein is over-expressed its inhibition by small molecule inhibitors in vitro in the absence of a BCR ligand, induced apoptosis. (See Contri A, J Clin Invest. 2005; 115:369-378). Corroborating these findings, in vitro treatment of CLL cells with R406 a small molecule inhibitor of Syk (a substrate of Lyn) also mediated an apoptotic response. (See Buchner M, Cancer Res. 2009; 69:5424-5432 and Gobessi S, Leukemia. 2009; 23:686-697). Both sets of data support a pivotal role for tonic signaling in CLL B-cell survival. Further insights into the relationship between BCR signaling, tonic signaling, phosphatase activity, and apoptotic response could be determined by measuring apoptosis in the presence of specific tyrosine phosphatase inhibitors specifically targeting SHP-1 and/or CD45. A priori, such inhibitors would be predicted to promote survival. Consistent with this hypothesis, ectopic expression of protein tyrosine phosphatase, PTPRO, (silenced in CLL by DNA methylation) increased growth inhibition in response to F-ara-A. See Motiwala T, Clin Cancer Res. 2007; 13:3174-3181.

Although not a member of the canonical BCR signaling network, the increase seen in p-STAT5(AUC 0.84, FIGS. 5 (A) (B)) could be due to a bystander effect resulting from phosphatase inhibition with consequent increase in kinase activities for which STAT5 is a substrate. Interestingly, Sattler et al, showed the importance of H₂O₂ generation with consequent increases in p-STAT5 in several hematopoietic growth factor cascades in cell lines. See Sattler M, Blood. 1999; 93:2928-2935. A pivotal role was also demonstrated for activated STAT5 in hematopoietic stem cell self renewal and expansion of multipotential progenitors in myeloid disease. It is tempting to speculate about such roles for activated STAT5 in CLL. See Kato Y, J Exp Med. 2005; 202:169-179. Furthermore, the dependence of hematological malignancies on p-STAT5 was shown in a recent study where phospholipase C-β3 was shown to be a tumor suppressor by acting as a scaffold for simultaneous interaction with p-STAT5 and SHP-1 and in so doing promoted the dephosphorylation of p-STAT5. See Xiao W, Cancer Cell. 2009; 16:161-171. Whether phospholipase C-0 3 plays a similar role in regulating p-STAT5 in CLL awaits further study.

The clinical complexity (and unpredictability) of CLL as well as the many components governing cell proliferation and survival mechanisms, suggest a diversity of mechanisms that give rise to CLL, Remarkably, however the current studies demonstrate a convergence of signaling patterns in CLL that lead to a remarkably limited set of phenotypic cell signaling outcomes. These regularized phenotypes, and the relationships between markers of their activity insofar as they exist in a self-regulating network, could be used to predict drug responses in vitro from signaling data within a ‘heterogeneous’ set of patient samples. This suggests there are only a limited number of pathway variations, despite the underlying heterogeneity, that maintain cellular homeostasis in CLL, and that numerous surrogates exist for pathway structures that ultimately drive apoptotic outcomes in response to therapy. Further studies will determine whether signaling profiles measured by this technology for individual samples can predict treatment outcome and stratify patients who might gain the most benefit from fludarabine-based treatment regimens.

1. A method for classifying a cell comprising
contacting said cell with a modulator, wherein the modulator is a tyrosine phosphatase inhibitor;
determining the presence or absence of a change in activation level of an activatable element in said cell; and
classifying said cell based on said presence or absence of said change in the activation level of said activatable element.

2. The method of claim 1, wherein said tyrosine phosphatase inhibitor is CD45-associated protein tyrosine phosphatase inhibitor, PHPS1, PP1, PP2, Bay U6751, BVT.948, NSC 295642, PRL-3 Inhibitor I, Phenylarsine oxide, Sodium Stibogluconate, Sodium orthovanadate, pervanadate, bisperoxovanadium, phenylarsine oxide, alendronate, etidronate, vanadate, gallium nitrate, suramin, or aplidin.

3. The method of claim 1, wherein said tyrosine phosphatase inhibitor is hydrogen peroxide (H₂O₂).

4. The method of claim 1 wherein said change in activation level of an activatable element is an increase in activation level of an activatable element.

5. The method of claim 1 wherein said cell is a cancer cell.

6. The method of claim 5 wherein said presence or absence of a change in activation level of said activatable element is compared to a normal cell contacted with said modulator.

7. The method of claim 1 wherein said cell is a hematopoietically derived cell.

8. The method of claim 1 wherein the presence or absence of a change in the activation levels of a plurality of activatable elements is determined in said determining step.

9. The method of claim 1 wherein said classification comprises classifying said cell as a cell that is correlated with a clinical outcome.

10. The method of claim 9 wherein said clinical outcome is the presence or absence of a neoplastic, autoimmune and/or a hematopoietic condition.

11. The method of claim 10 wherein said neoplastic, autoimmune and/or hematopoietic condition is selected from the group consisting of Non-Hodgkin Lymphoma, Hodgkin or other lymphomas, acute or chronic leukemias, polycythemias, thrombocythemias, multiple myeloma, plasma cell disorders, myelodysplastic disorders, myeloproliferative disorders, myelofibrosis, atypical immune lymphoproliferations, systemic lupus erythematosis (SLE) and rheumatoid arthritis (RA).

12. The method of claim 10 wherein said neoplastic, autoimmune and/or hematopoietic condition is a non-B lineage derived disorder selected from the group consisting of acute myeloid leukemia (AML), Chronic Myeloid Leukemia (CML), non-B cell acute lymphocytic leukemia (ALL), non-B cell lymphomas, myelodysplastic disorders, myeloproliferative disorders, myelofibrosis, thrombocythemias, and non-B atypical immune lymphoproliferations.

13. The method of claim 10 wherein said neoplastic, autoimmune and/or hematopoietic condition is a B-Cell or B cell lineage derived disorder selected from the group consisting of Chronic Lymphocytic Leukemia (CLL), B-cell lymphoma, B lymphocyte lineage leukemia, B lymphocyte lineage lymphoma, Multiple Myeloma, acute lymphoblastic leukemia (ALL), B-cell pro-lymphocytic leukemia, precursor B lymphoblastic leukemia, hairy cell leukemia and plasma cell disorders.

14. The method of claim 13 wherein said neoplastic, autoimmune and/or hematopoietic condition is CLL.

15. The method of claim 9 wherein said clinical outcome is the staging or grading of a neoplastic, autoimmune and/or hematopoietic condition.

16. The method of claim 1 wherein said classification further comprises determining a method of treatment.

17. The method of claim 1 wherein activation level is selected from the group consisting of cleavage by extracellular or intracellular protease exposure, novel hetero-oligomer formation, glycosylation level, phosphorylation level, acetylation level, methylation level, biotinylation level, glutamylation level, glycylation level, hydroxylation level, isomerization level, prenylation level, myristoylation level, lipoylation level, phosphopantetheinylation level, sulfation level, ISGylation level, nitrosylation level, palmitoylation level, SUMOylation level, ubiquitination level, neddylation level, citrullination level, deamidation level, disulfide bond formation level, proteolytic cleavage level, translocation level, changes in protein turnover, multi-protein complex level e, oxidation level, multi-lipid complex, and biochemical changes in cell membrane.

18. The method of claim 1 wherein said activatable element is a protein subject to phosphorylation or dephosphorylation.

19. The method of claim 18 wherein said protein is selected from the group consisting of PI3-Kinase (p85, p110a, p110b, p110d), Jak1, Jak2, SOCs, Rac, Rho, Cdc42, Ras-GAP, Vav, Tiam, Sos, Dbl, Nck, Gab, PRK, SHP1, and SHP2, SHIP1, SHIP2, sSHIP, PTEN, Shc, Grb2, PDK1, SGK, Akt1, Akt2, Akt3, TSC1,2, Rheb, mTor, 4EBP-1, p70S6Kinase, S6, LKB-1, AMPK, PFK, Acetyl-CoAa Carboxylase, DokS, Rafs, Mos, Tp12, MEK1/2, MLK3, TAK, DLK, MKK3/6, MEKK1,4, MLK3, ASK1, MKK4/7, SAPK/JNK1,2,3, p38s, Erk1/2, Syk, Btk, BLNK, LAT, ZAP70, Lck, Cbl, SLP-76, PLCγ1, PLCγ2, transcription factor, STAT1, STAT5, STAT4, STAT5, STAT6, FAK, p130CAS, PAKs, LIMK1/2, Hsp90, Hsp70, Hsp27, SMADs, Rel-A (p65-NFκB), CREB, Histone H₂B, HATs, HDACs, PKR, Rb, Cyclin D, Cyclin E, Cyclin A, Cyclin B, P16, p14Arf, p27KIP, p21CIP, Cdk4, Cdk6, Cdk7, Cdk1, Cdk2, Cdk9, Cdc25, A/B/C, Abl, E2F, FADD, TRADD, TRAF2, RIP, Myd88, BAD, Bcl-2, Mcl-1, Bcl-XL, Caspase 2, Caspase 3, Caspase 6, Caspase 7, Caspase 8, Caspase 9, cleaved caspase 3, cleaved caspase 8, cytosolic cytochrome c, PARP, cleaved PARP, IAPs, Smac, Fodrin, Actin, Src, Lyn, Fyn, Lck, NIK, IxB, p65(RelA), IKKα, PKA, PKCα, PKCβ, PKCθ, PKCδ, CAMK, Elk, AFT, Myc, Egr-1, NFAT, ATF-2, Mdm2, p53, DNA-PK, Chk1, Chk2, ATM, ATR, β-catenin, CrkL, GSK3α, GSK3β, and FOXO.

20. The method of claim 18 wherein said protein is Lyn, Syk, PLCγ2, BLNK, STAT5, Erk1/2, p65(RelA), Akt1, Akt2, Akt3, S6, Chk2, cleaved PARP, cleaved caspase 3, cleaved caspase 8, cytosolic cytochrome C or Bcl-2.

21. The method of claim 20 wherein said protein is STAT5, Akt1, Akt2 or Akt3.

22. The method of claim 1, further comprising contacting said cell with a B cell receptor cross-linker.

23. The method of claim 22, wherein the cross-linker is F(ab)2 IgM, IgG, IgD, polyclonal BCR antibodies, monoclonal BCR antibodies, Fc receptor derived binding elements, Polyclonal IgM antibodies, Monoclonal IgM antibodies, Biotinylated F(ab)₂ IgG/M, Biotinylated Polyclonal IgM antibodies and Biotinylated Monoclonal IgM antibodies.

24. The method of claim 1, wherein said classifying is performed using a classification method.

25. The method of claim 24, wherein said classification method is logistic regression, decision tree, random forest, support vector machine, boosting or receiver operating characteristic (ROC) curve.

26. A method for classifying a cell comprising
contacting said cell with a modulator;
determining the presence or absence of a change in activation level of an activatable element in said cell, and
classifying said cell as apoptotic refractory or competent based on said presence or absence of said change in the activation level of said activatable element.

27. A method for classifying a cell comprising
contacting said cell with a modulator, wherein the modulator is a tyrosine phosphatase inhibitor;
determining the presence or absence of a change in activation level of at least two activatable elements of Lyn, Syk, BLNK, PLCγ2, Erk or STAT5 in said cell; and
classifying said cell based on said presence or absence of said change in the activation level of said activatable element.

28. A method of determining the presence or absence of a condition in an individual comprising
subjecting a cell from said individual to a modulator, wherein the modulator is a tyrosine phosphatase inhibitor,
determining the activation level of an activatable element in said cell, and
determining the presence or absence of said condition based on said activation level.

29. A method of determining tonic signaling status of a cell comprising
subjecting said cell to a modulator, wherein the modulator is a tyrosine phosphatase inhibitor;
determining the activation level of an activatable element that participates in a tonic signaling pathway in said cell, and
determining the status of a tonic signaling pathway in said cell from said activation level.

30. A method of correlating and/or classifying an activation state of a CLL cell with a clinical outcome in an individual comprising
subjecting said CLL cell from said individual to a modulator, wherein said CLL cell comprises a B-Cell receptor (BCR), and wherein the modulator is a tyrosine phosphatase inhibitor;
determining the activation levels of a plurality of activatable elements; and
identifying a pattern of said activation levels of said plurality of activatable elements to determine the presence or absence of an alteration in signaling proximal to the BCR, wherein the presence of said alteration is indicative of a clinical outcome.

31. A method of determining a phenotypic profile of a population of cells comprising
exposing the population of cells to a plurality of modulators in separate cultures, wherein at least one of the modulators is a tyrosine phosphatase inhibitor,
determining the presence or absence of a change in activation level of an activatable element in said cell population from each of said separate culture
classifying said cell population based on said presence or absence of said change in the activation of said activatable element from each of said separate culture.

32. A method of classifying a cell population comprising
contacting said cell population with at least one modulator, where the modulator is F(ab)2 IgM, anti-CD20 antibody, anti-CD52 antibody, anti-CD22 antibody, anti-CD23 antibody, H₂O₂, PMA, BAFF, April, SDF1a, CD40L, IGF-1, Imiquimod, polyCpG, fludarabine, cyclophosphamide, chlorambucil, IL-7, IL-6, IL-10, IL-27, IL-4, IL-2, IL-3, thapsigargin or a combination thereof,
determining the presence or absence of a change in activation level of an activatable element in said cell population, and
classifying said cell population based on levels of apoptotic refractory or competent cells in said cell population.
